(19) United States
(12) Patent Application Publication
(10) Pub. No.: US 2025/0043358 A1
(43) Pub. Date: Feb. 6, 2025

(54) NOVEL GENE CLASSIFIERS AND USES THEREOF IN NON-MELANOMA SKIN CANCERS

(71) Applicant: DermTech, LLC, San Diego, CA (US)

(72) Inventors: John Daniel Dobak, III, La Jolla, CA (US); Burkhard Jansen, San Diego, CA (US); Zuxu Yao, San Diego, CA (US)

(21) Appl. No.: 18/602,952

(22) Filed: Mar. 12, 2024

Related U.S. Application Data
(63) Continuation of application No. 16/969,526, filed on Aug. 12, 2020, now Pat. No. 11,976,332.
(60) Provisional application No. 62/630,627, filed on Feb. 14, 2018.

(51) Int. Cl. C12Q 1/6886

(52) U.S. Cl. CPC C12Q 1/6886; C12Q 2600/106; C12Q 2600/156; C12Q 2600/158

(57) ABSTRACT

Described herein are methods, systems, and compositions for non-invasively diagnosing or detecting a skin disease or disorder. Diagnosing or detecting a non-melanoma skin cancer as provided herein comprises detecting gene expression levels of a set of identified genes and in some instances further detecting mutations in a gene of interest.

### CROSS-REFERENCE

This is a continuation application of U.S. application Ser. No. 16/969,526, filed Aug. 12, 2020, which is U.S. National Phase of International Application No. PCT/US2019/018102, filed Feb. 14, 2019, which claims the benefit of U.S. Provisional Application No. 62/630,627 filed Feb. 14, 2018, which is incorporated herein by reference in its entirety.

### BACKGROUND

Skin diseases are some of the most common human illnesses and represent an important global burden in healthcare. Three skin diseases are in the top ten most prevalent diseases worldwide, and eight fall into the top 50. When considered collectively, skin conditions range from being the second to the 11th leading causes of years lived with disability.

### SUMMARY

An aspect described herein is a method of detecting gene expression levels of at least two of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, and SPP1 in a subject in need thereof, comprising: (a) isolating nucleic acids from a biological sample obtained from the subject; and (b) detecting the expression levels of the at least two of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, and SPP1 by contacting the isolated nucleic acids with a set of probes that recognizes the at least two of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, and SPP1, and detects binding between the at least two of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, and SPP1 and the set of probes. In one feature, the set of probes recognizes at least three genes selected from the group consisting of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, and SPP1. In one feature, the set of probes recognizes at least four genes selected from the group consisting of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, and SPP1. In one feature, the set of probes recognizes at least five genes selected from the group consisting of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, and SPP1. In one feature, the set of probes recognizes at least six genes selected from the group consisting of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, and SPP1. In one feature, the set of probes recognizes at least seven genes selected from the group consisting of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, and SPP1. In one feature, the set of probes recognizes: MMP1, S100A7, CMPK2, IRF7, IGFL1, CXCL1, UPP1, DEFB4A, FOS, OAS3, SCD5, RTP4, and VEGFA; MMP1, S100A7, CMPK2, IRF7, IGFL1, CXCL1, and UPP1; or MMP1, S100A7, CMPK2, IRF7, IGFL1, and CXCL1. In one feature, the set of probes recognizes at least two genes selected from the group consisting of MMP1, S100A7, CMPK2, IRF7, IGFL1, CXCL1, and UPP1. In one feature, the set of probes recognizes at least three genes, at least four genes, at least five genes, or at least six genes selected from the group consisting of MMP1, S100A7, CMPK2, IRF7, IGFL1, CXCL1, and UPP1. In one feature, the set of probes recognizes at least two genes selected from the group consisting of MMP1, S100A7, CMPK2, IRF7, IGFL1, and CXCL1. In one feature, the set of probes recognizes at least three genes, at least four genes, or at least five genes selected from the group consisting of MMP1, S100A7, CMPK2, IRF7, IGFL1, and CXCL1. In one feature, the set of probes recognizes at least two genes selected from the group consisting of SCD5, S100A7, CMPK2, and IRF7. In one feature, the set of probes recognizes at least two and no more than thirteen genes selected from: MMP1, S100A7, CMPK2, IRF7, IGFL1, CXCL1, UPP1, DEFB4A, FOS, OAS3, SCD5, RTP4, and VEGFA. In one feature, the method further comprises detecting a mutational change of at least one gene of interest. In one feature, the detecting comprises allele specific polymerase chain reaction (PCR) or a sequencing reaction. In one feature, wherein the at least one gene of interest comprises TERT, CDKN2A, TP53, or PTCH1. In one feature, a mutation in TP53 translates to amino acid positions in TP53 selected from: R175, S240, G245, R248, R249, R273, R282, or T284, wherein the numbering of amino acid residues corresponds to SEQ ID NO: 1. In one feature, a mutation in TP53 is in exon 1, 2, 3, 4, 5, 6, 7, 8, 9, 10, 11, or a combination thereof of TP53. In one feature, a mutation in TP53 is in exon 5, 7, 8, or a combination thereof of TP53. In one feature, a mutation in PTCH1 is in exon 1, 2, 3, 4, 5, 6, 7, 8, 9, 10, 11, 12, 13, 14, 15, 16, 17, 18, 19, 20, 21, 22, 23, 24, or a combination thereof of PTCH1. In one feature, a mutation in PTCH1 is in exon 14, 15, 17, 23, or a combination thereof of PTCH1. In one feature, a mutation in CDKN2A is in exon 1, 2, 3, 4, 5, 6, 7, 8, or a combination thereof of CDKN2A. In one feature, a mutation in TERT is in exon 1, 2, 3, 4, 5, 6, 7, 8, 9, 10, 11, 12, 13, 14, 15, 16, or a combination thereof of TERT. In one feature, a mutation in TERT is in a promoter region of TERT. In one feature, the mutational change comprises at least 1.5×, 2×, 3×, 4×, 5×, 6×, 7×, 8×, 9×, 10×, 11×, or 12× more mutations in TERT, CDKN2A, TP53, PTCH1, or a combination thereof, compared to a normal biological sample. In one feature, the mutational change comprises at least 10%, 15%, 20%, 25%, 30%, 35%, 40%, 45%, 50%, 55%, 60%, 65%, 70%, 75%, or 80% more mutations in TERT, CDKN2A, TP53, PTCH1, or a combination thereof, compared to anormal biological sample. In one feature, the subject is suspected of having a cancer. In one feature, the subject is suspected of having a skin cancer. In one feature, isolating the nucleic acids comprises using a plurality of beads. In one feature, the plurality of beads is a plurality of silica-coated beads. In one feature, the plurality of silica-coated beads is a plurality of silica-coated magnetic beads. In one feature, the biological sample comprises a blood sample, saliva sample, urine sample, serum sample, plasma sample, tear sample, skin sample, tissue sample, hair sample, sample from cellular extracts, or a tissue biopsy sample. In one feature, the biological sample comprises a skin sample. In one feature, the skin sample comprises a lesion, and wherein the lesion is suspected to be melanoma, lupus, rubeola, acne, hemangioma, psoriasis, eczema, candidiasis, impetigo, shingles, leprosy, Crohn's disease, inflammatory dermatoses, bullous diseases, infections, basal cell carcinoma, actinic keratosis, seborrheic keratosis, merkel cell carcinoma, sebaceous carcinoma, squamous cell carcinoma, or dermatofibrosarcoma protuberans. In one feature, the lesion is suspected to be basal cell carcinoma or squamous cell carcinoma. In one feature, the skin sample comprises keratinocytes, melanocytes, basal cells, T-cells, or dendritic cells. In one feature, the skin sample is obtained by applying a plurality of adhesive patches to the skin sample in a manner sufficient to adhere the skin sample to the adhesive patch, and removing the adhesive patch from the skin in a manner sufficient to retain the adhered skin sample to the adhesive patch. In one feature, the plurality of adhesive patches comprises at least 4 adhesive patches. In one feature, the plurality of adhesive patches comprises about 4 adhesive patches. In one feature, the skin sample is obtained by pooling the plurality of adhesive patches. In one feature, each adhesive patch of the plurality of adhesive patches is used separately. In one feature, each adhesive patch of the plurality of adhesive patches is circular. In one feature, the each adhesive patch is at least 19 mm in diameter. In one feature, the each adhesive patch is about 19 mm in diameter. In one feature, an effective amount of the skin sample is removed by the plurality of adhesive patches. In one feature, the effective amount comprises between about 50 microgram to about 500 microgram, between about 100 microgram to about 450 microgram, between about 100 microgram to about 350 microgram, between about 100 microgram to about 300 microgram, between about 120 microgram to about 250 microgram, or between about 150 microgram to about 200 microgram of the nucleic acids. In one feature, the nucleic acids are stable on the plurality of adhesive patches for at least 1 week. In one feature, the nucleic acids are stable on the plurality of adhesive patches at a temperature of up to about 60° C. In one feature, the nucleic acids are stable on the plurality of adhesive patches at room temperature. In one feature, a yield of the nucleic acids is at least about 200 picograms, at least about 500 picograms, at least about 750 picograms, at least about 1000 picograms, at least about 1500 picograms, or at least about 2000 picograms. In one feature, the nucleic acids comprise RNA, DNA, or a combination thereof. In one feature, the RNA is mRNA. In one feature, the RNA is cell-free circulating RNA. In one feature, the DNA is genomic DNA. In one feature, the genomic DNA is cell-free circulating genomic DNA. In one feature, detecting the expression levels comprise quantitative polymerase chain reaction (qPCR), sequencing, or microarray analysis.

An aspect described herein is a method of detecting gene expression levels of at least two of MMP1, S100A7, CMPK2, IRF7, IGFL1, CXCL1, and UPP1 in a subject in need thereof, comprising: (a) isolating nucleic acids from a biological sample obtained from the subject; and (b) detecting the expression levels of the at least two of MMP1, S100A7, CMPK2, IRF7, IGFL1, CXCL1, and UPP1, by contacting the isolated nucleic acids with a set of probes that recognizes the at least two of MMP1, S100A7, CMPK2, IRF7, IGFL1, CXCL1, and UPP1, and detects binding between the at least two of MMP1, S100A7, CMPK2, IRF7, IGFL1, CXCL1, and UPP1 and the set of probes. In one feature, the set of probes recognizes at least three genes, at least four genes, at least five genes, or at least six genes selected from the group consisting of MMP1, S100A7, CMPK2, IRF7, IGFL1, CXCL1, and UPP1. In one feature, the set of probes recognizes at least two genes selected from the group consisting of MMP1, S100A7, CMPK2, IRF7, IGFL1, and CXCL1. In one feature, the set of probes recognizes at least three genes, at least four genes, or at least five genes selected from the group consisting of MMP1, S100A7, CMPK2, IRF7, IGFL1, and CXCL1. In one feature, the set of probes recognizes at least two genes selected from the group consisting of SCD5, S100A7, CMPK2, and IRF7. In one feature, the set of probes recognizes at least two and no more than seven genes selected from: MMP1, S100A7, CMPK2, IRF7, IGFL1, CXCL1, and UPP1. In one feature, the method further comprises detecting a mutational change of at least one gene of interest. In one feature, the detecting comprises allele specific polymerase chain reaction (PCR) or a sequencing reaction. In one feature, the at least one gene of interest comprises TERT, CDKN2A, TP53, or PTCH1. In one feature, a mutation in TP53 translates to amino acid positions in TP53 selected from: R175, S240, G245, R248, R249, R273, R282, or T284, wherein the numbering of amino acid residues corresponds to SEQ ID NO: 1. In one feature, a mutation in TP53 is in exon 1, 2, 3, 4, 5, 6, 7, 8, 9, 10, 11, or a combination thereof of TP53. In one feature, a mutation in TP53 is in exon 5, 7, 8, or a combination thereof of TP53. In one feature, a mutation in PTCH1 is in exon 1, 2, 3, 4, 5, 6, 7, 8, 9, 10, 11, 12, 13, 14, 15, 16, 17, 18, 19, 20, 21, 22, 23, 24, or a combination thereof of PTCH1. In one feature, a mutation in PTCH1 is in exon 14, 15, 17, 23, or a combination thereof of PTCH1. In one feature, a mutation in CDKN2A is in exon 1, 2, 3, 4, 5, 6, 7, 8, or a combination thereof of CDKN2A. In one feature, a mutation in TERT is in exon 1, 2, 3, 4, 5, 6, 7, 8, 9, 10, 11, 12, 13, 14, 15, 16, or a combination thereof of TERT. In one feature, a mutation in TERT is in a promoter region of TERT. In one feature, the mutational change comprises at least 1.5×, 2×, 3×, 4×, 5×, 6×, 7×, 8×, 9×, 10×, 11×, or 12× more mutations in TERT, CDKN2A, TP53, PTCH1, or a combination thereof, compared to a normal biological sample. In one feature, the mutational change comprises at least 10%, 15%, 20%, 25%, 30%, 35%, 40%, 45%, 50%, 55%, 60%, 65%, 70%, 75%, or 80% more mutations in TERT, CDKN2A, TP53, PTCH1, or a combination thereof, compared to a normal biological sample. In one feature, the subject is suspected of having a cancer. In one feature, the subject is suspected of having a skin cancer. In one feature, isolating the nucleic acids comprises using a plurality of beads. In one feature, plurality of beads is a plurality of silica-coated beads. In one feature, the plurality of silica-coated beads is a plurality of silica-coated magnetic beads. In one feature, the biological sample comprises a blood sample, saliva sample, urine sample, serum sample, plasma sample, tear sample, skin sample, tissue sample, hair sample, sample from cellular extracts, or a tissue biopsy sample. In one feature, the biological sample comprises a skin sample. In one feature, the skin sample comprises a lesion, and wherein the lesion is suspected to be melanoma, lupus, rubeola, acne, hemangioma, psoriasis, eczema, candidiasis, impetigo, shingles, leprosy, Crohn's disease, inflammatory dermatoses, bullous diseases, infections, basal cell carcinoma, actinic keratosis, seborrheic keratosis, merkel cell carcinoma, sebaceous carcinoma, squamous cell carcinoma, or dermatofibrosarcoma protuberans. In one feature, the lesion is suspected to be basal cell carcinoma or squamous cell carcinoma. In one feature, the skin sample comprises keratinocytes, melanocytes, basal cells, T-cells, or dendritic cells. In one feature, the skin sample is obtained by applying a plurality of adhesive patches to the skin sample in a manner sufficient to adhere the skin sample to the adhesive patch, and removing the adhesive patch from the skin in a manner sufficient to retain the adhered skin sample to the adhesive patch. In one feature, the plurality of adhesive patches comprises at least 4 adhesive patches. In one feature, the plurality of adhesive patches comprises about 4 adhesive patches. In one feature, the skin sample is obtained by pooling the plurality of adhesive patches. In one feature, each adhesive patch of the plurality of adhesive patches is used separately. In one feature, each adhesive patch of the plurality of adhesive patches is circular. In one feature, the each adhesive patch is at least 19 mm in diameter. In one feature, the each adhesive patch is about 19 mm in diameter. In one feature, an effective amount of the skin sample is removed by the plurality of adhesive patches. In one feature, the effective amount comprises between about 50 microgram to about 500 microgram, between about 100 microgram to about 450 microgram, between about 100 microgram to about 350 microgram, between about 100 microgram to about 300 microgram, between about 120 microgram to about 250 microgram, or between about 150 microgram to about 200 microgram of the nucleic acids. In one feature, the nucleic acids are stable on the plurality of adhesive patches for at least 1 week. In one feature, the nucleic acids are stable on the plurality of adhesive patches at a temperature of up to about 60° C. In one feature, the nucleic acids are stable on the plurality of adhesive patches at room temperature. In one feature, a yield of the nucleic acids is at least about 200 picograms, at least about 500 picograms, at least about 750 picograms, at least about 1000 picograms, at least about 1500 picograms, or at least about 2000 picograms. In one feature, the nucleic acids comprise RNA, DNA, or a combination thereof. In one feature, the RNA is mRNA. In one feature, the RNA is cell-free circulating RNA. In one feature, the DNA is genomic DNA. In one feature, the genomic DNA is cell-free circulating genomic DNA. In one feature, detecting the expression levels comprise quantitative polymerase chain reaction (qPCR), sequencing, or microarray analysis.

An aspect described herein is a method of diagnosing and treating a disease or disorder in a subject, comprising: (a) isolating nucleic acids from a biological sample obtained from the subject; (b) detecting the expression levels of at least two genes selected from the group consisting of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, and SPP1, and comparing the expression levels of the at least two genes to a control; (c) identifying the subject as having the disease or disorder if there is a change in the expression levels of the at least two genes relative to the control; and (d) administering an effective amount of a therapeutic agent to the diagnosed subject.

An aspect described herein is a method of diagnosing a disease or disorder in a subject, comprising: (a) isolating nucleic acids from a biological sample obtained from the subject; (b) detecting the expression levels of at least two genes selected from the group consisting of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, and SPP1, and comparing the expression levels of the at least two genes to a control; and (c) identifying the subject as having the disease or disorder if there is a change in the expression levels of the at least two genes relative to the control. In one feature, at least three genes are selected from the group consisting of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, and SPP1. In one feature, at least four genes are selected from the group consisting of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, and SPP1. In one feature, at least five genes are selected from the group consisting of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, and SPP1. In one feature, at least six genes are selected from the group consisting of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, and SPP1. In one feature, at least seven genes are selected from the group consisting of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, and SPP1. In one feature, the at least two genes are selected from the group consisting of MMP1, S100A7, CMPK2, IRF7, IGFL1, CXCL1, and UPP1. In one feature, at least three genes, at least four genes, at least five genes, or at least six genes are selected from the group consisting of MMP1, S100A7, CMPK2, IRF7, IGFL1, CXCL1, and UPP1. In one feature, the at least two genes is selected from the group consisting of MMP1, S100A7, CMPK2, IRF7, IGFL1, and CXCL1. In one feature, at least three genes, at least four genes, or at least five genes are selected from the group consisting of MMP1, S100A7, CMPK2, IRF7, IGFL1, and CXCL1. In one feature, the at least two genes is selected from the group consisting of SCD5, S100A7, CMPK2, and IRF7. In one feature, the method further comprises detecting a mutational change of a gene of interest. In one feature, the detecting comprises allele specific polymerase chain reaction (PCR) or a sequencing reaction. In one feature, the gene of interest comprises TERT, CDKN2A, TP53, or PTCH1. In one feature, a mutation in TP53 translates to amino acid positions in TP53 selected from: R175, S240, G245, R248, R249, R273, R282, or T284, wherein the numbering of amino acid residues corresponds to SEQ ID NO: 1. In one feature, a mutation in TP53 is in exon 1, 2, 3, 4, 5, 6, 7, 8, 9, 10, 11, or a combination thereof of TP53. In one feature, a mutation in TP53 is in exon 5, 7, 8, or a combination thereof of TP53. In one feature, a mutation in PTCH1 is in exon 1, 2, 3, 4, 5, 6, 7, 8, 9, 10, 11, 12, 13, 14, 15, 16, 17, 18, 19, 20, 21, 22, 23, 24, or a combination thereof of PTCH1. In one feature, a mutation in PTCH1 is in exon 14, 15, 17, 23, or a combination thereof of PTCH1. In one feature, a mutation in CDKN2A is in exon 1, 2, 3, 4, 5, 6, 7, 8, or a combination thereof of CDKN2A. In one feature, a mutation in TERT is in exon 1, 2, 3, 4, 5, 6, 7, 8, 9, 10, 11, 12, 13, 14, 15, 16, or a combination thereof of TERT. In one feature, a mutation in TERT is in a promoter region of TERT In one feature, the mutational change comprises at least 1.5×, 2×, 3×, 4×, 5×, 6×, 7×, 8×, 9×, 10×, 11×, or 12× more mutations in TERT CDKN2A, TP53, PTCH1, or a combination thereof, compared to a normal biological sample. In one feature, the mutational change comprises at least 10%, 15%, 20%, 25%, 30%, 35%, 40%, 45%, 50%, 55%, 60%, 65%, 70%, 75%, or 80% more mutations in TERT, CDKN2A, TP53, PTCH1, or a combination thereof, compared to a normal biological sample. In one feature, the subject is suspected of having a cancer. In one feature, the subject is suspected of having a skin cancer. In one feature, the biological sample comprises a blood sample, saliva sample, urine sample, serum sample, plasma sample, tear sample, skin sample, tissue sample, hair sample, sample from cellular extracts, or a tissue biopsy sample. In one feature, the biological sample comprises a skin sample. In one feature, the skin sample comprises a lesion, and wherein the lesion is suspected to be melanoma, lupus, rubeola, acne, hemangioma, psoriasis, eczema, candidiasis, impetigo, shingles, leprosy, Crohn's disease, inflammatory dermatoses, bullous diseases, infections, basal cell carcinoma, actinic keratosis, seborrheic keratosis, merkel cell carcinoma, sebaceous carcinoma, squamous cell carcinoma, or dermatofibrosarcoma protuberans. In one feature, the lesion is suspected to be basal cell carcinoma or squamous cell carcinoma. In one feature, treatment for the basal cell carcinoma or squamous cell carcinoma comprises surgery. In one feature, treatment for actinic keratosis comprises a topical treatment. In one feature, the skin sample comprises keratinocytes, melanocytes, basal cells, T-cells, or dendritic cells. In one feature, the skin sample is obtained by applying a plurality of adhesive patches to the skin sample in a manner sufficient to adhere the skin sample to the adhesive patch, and removing the adhesive patch from the skin in a manner sufficient to retain the adhered skin sample to the adhesive patch. In one feature, the plurality of adhesive patches comprises at least 4 adhesive patches. In one feature, the plurality of adhesive patches comprises about 4 adhesive patches. In one feature, the skin sample is obtained by pooling the plurality of adhesive patches. In one feature, each adhesive patch of the plurality of adhesive patches is used separately. In one feature, each adhesive patch of the plurality of adhesive patches is circular. In one feature, the each adhesive patch is at least 19 mm in diameter. In one feature, the each adhesive patch is about 19 mm in diameter. In one feature, an effective amount of the skin sample is removed by the plurality of adhesive patches. In one feature, the effective amount comprises between about 50 microgram to about 500 microgram, between about 100 microgram to about 450 microgram, between about 100 microgram to about 350 microgram, between about 100 microgram to about 300 microgram, between about 120 microgram to about 250 microgram, or between about 150 microgram to about 200 microgram of the nucleic acids. In one feature, the nucleic acids are stable on the plurality of adhesive patches for at least 1 week. In one feature, the nucleic acids are stable on the plurality of adhesive patches at a temperature of up to about 60° C. In one feature, the nucleic acids are stable on the plurality of adhesive patches at room temperature. In one feature, a yield of the nucleic acids is at least about 200 picograms, at least about 500 picograms, at least about 750 picograms, at least about 1000 picograms, at least about 1500 picograms, or at least about 2000 picograms. In one feature, the nucleic acids comprise RNA, DNA, or a combination thereof. In one feature, the RNA is mRNA. In one feature, the RNA is cell-free circulating RNA. In one feature, the DNA is genomic DNA. In one feature, the genomic DNA is cell-free circulating genomic DNA. In one feature, detecting the expression levels comprises quantitative polymerase chain reaction (qPCR), sequencing, or microarray analysis.

An aspect described herein is a computer-implemented method for differentiating one or more cancer samples from one or more non-cancer samples, comprising: (a) hybridizing a set of probes that recognizes MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, and SPP1 to generate gene expression data; (b) obtaining, by a processor, the gene expression data; and (c) analyzing, by the processor, the gene expression data to differentiate the one or more cancer samples from the one or more non-cancer samples, wherein the analysis comprises: (i) generating a plurality of pair-wise interactions between at least two genes of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, and SPP1 in a set of samples; (ii) analyzing the plurality of pair-wise interactions using a machine learning method to determine an area under a curve (AUC) value for each of the plurality of pair-wise interactions; and (iii) differentiating the one or more cancer samples from the one or more non-cancer samples when an AUC value is greater than above about 0.8.

An aspect described herein are methods, wherein a sensitivity of the methods is at least 80%.

An aspect described herein are methods, wherein a specificity of the method is at least 80%.

An aspect described herein is a computer-implemented system comprising: (a) a first computing device comprising a processor, a memory module, an operating system, and a computer program including instructions executable by the processor to create a data acquisition application for receiving gene expression from a sample, the data acquisition application comprising: a data receiving module for receiving gene expression data of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, SPP1, or a combination thereof; and (b) a second computing device comprising a processor, a memory module, an operating system, and a computer program including instructions executable by the processor to create a data analysis application for differentiating a cancer sample from a non-cancer sample, the data analysis application comprising a data analysis module to: (i) generate a plurality of pair-wise interactions between at least two genes of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, and SPP1 from the gene expression data; and (ii) analyze the plurality of pair-wise interactions with a control using a machine learning method to differentiate a cancer sample from a non-cancer sample, wherein the machine learning method comprises: (1) identifying a plurality of weights associated with the plurality of pair-wise interactions based on a top score; (2) determining an area under a curve (AUC) value for each of the plurality of pair-wise interactions; and (3) classifying the sample as a cancer or non-cancer sample based on the plurality of pair-wise interactions and the plurality of weights when the AUC value is greater than about 0.8.

An aspect described herein is a non-transitory computer-readable medium with instructions stored thereon, that when executed by a processor, perform the steps comprising: (a) generating gene expression data of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, and SPP1 from a set of biological samples by a hybridizing method; (b) obtaining, by a processor, the gene expression data; (c) generating a plurality of pair-wise interactions between at least two genes of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, and SPP1; (d) analyzing the plurality of pair-wise interactions using a machine learning method to determine an area under a curve (AUC) value for each of the plurality of pair-wise interactions; and (e) differentiating the one or more cancer samples from the one or more non-cancer samples when an AUC value is greater than above about 0.8.

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 illustrates a chart showing exemplary subtypes of basal cell carcinoma (BCC).

FIG. 2A-FIG. 2B illustrate graphs comparing various algorithmic models. FIG. 2A shows algorithmic models for a comparison of basal cell carcinoma (BCC) and squamous cell carcinoma (SCC) to actinic keratosis (AK), seborrheic keratosis (SK), and normal samples. FIG. 2B shows algorithmic models for comparison of basal cell carcinoma (BCC) and squamous cell carcinoma (SCC) to actinic keratosis (AK).

FIG. 3A-FIG. 3B illustrate graphs of an assay area under a curve (AUC) from the random forest (rf) analysis model. FIG. 3A shows rf analysis model for a comparison of basal cell carcinoma (BCC) and squamous cell carcinoma (SCC) to actinic keratosis (AK), seborrheic keratosis (SK), and normal samples. FIG. 3B shows rf analysis model for a comparison of basal cell carcinoma (BCC) and squamous cell carcinoma (SCC) to actinic keratosis (AK).

FIG. 4 shows algorithmic models for comparison of basal cell carcinoma (BCC) and squamous cell carcinoma (SCC) to actinic keratosis (AK), seborrheic keratosis (SK), and normal samples.

FIG. 5A-FIG. 5E show the boxplots of IGFL1, COL5A2 (FIG. 5A); IL24, AADACL2 (FIG. 5B); PTCH1, CD68 (FIG. 5C); PRKACA, SPP1 (FIG. 5D); or AADACL2, MMP1 (FIG. 5E) from Table 3.

FIG. 6A-FIG. 6C illustrate the sensitivity and specificity of the tested BCC and SCC samples. FIG. 6B illustrates the set of variables used to generate the AUC in FIG. 6A. FIG. 6C provides the sensitivity and specificity of the set of variables from FIG. 6B based on five different threshold criteria.

FIG. 7A shows an exemplary biplex PCR amplification of CDKN2A and TERT exons.

FIG. 7B shows an exemplary Sanger sequencing of CDKN2A and TERT wild-type sequences.

FIGS. 8A and 8B illustrate the mutation analysis of exemplary exons in PTCH1. FIG. 8A shows the number of mutations detected with respect to each exon from BCC, SCC, and AK. FIG. 8B shows the number of exons detected and the number of mutations detected within each exon in the tested BCC samples.

FIG. 9 illustrates the PCR amplification of exon 14, exon 15, and exon 23.

FIG. 10A illustrates the PCR amplification of exon 5, exon 7, and exon 8.

FIG. 10B shows the mutation location within each exon sequence.

FIG. 11 illustrates total RNA and gDNA obtained from the tested eluents.

FIG. 12 illustrates gDNA and total RNA extraction utilizing a 100 μL DT MB:30 μL Zymo MB ratio compared to the control, which contains 100 μL of DT MB.

FIG. 13A-FIG. 13B show 4 exemplary follow-up studies comparing the recovery of RNA (FIG. 13A) and gDNA (FIG. 13B) between DT MB, Zymo MB, and a mixture of DT MB and Zymo MB.

FIG. 14A shows PCR amplification of multiplex BRAF and NRAS amplicons (top) and TERT amplicon (bottom) from gDNA in isolated nucleic acid (NA) samples.

FIG. 14B shows chromatograms of BRAF V600 and 469 amplicons from the 8 samples, 4 isolated using DT only bead and 4 isolated using mixed beads.

FIG. 14C shows chromatograms of NRAS 12_13 and 61 amplicons from the 8 samples, 4 isolated using DT only bead and 4 isolated using mixed beads.

FIG. 14D shows chromatograms of TERT amplicons from the 8 samples, 4 isolated using DT only bead and 4 isolated using mixed beads.

### DETAILED DESCRIPTION

Non-melanoma skin cancer (NMSC) encompasses a collection of skin cancers that is not melanoma and is the most common type of skin cancer. NMSC includes angiosarcoma, basal cell carcinoma (BCC), cutaneous B-cell lymphoma, cutaneous T-cell lymphoma, dermatofibrosarcoma protuberans, Merkel cell carcinoma, sebaceous carcinoma, and squamous cell carcinoma of the skin (SCC). In some instances, basal cell carcinoma and squamous cell carcinoma are the two most common types of NMSC.

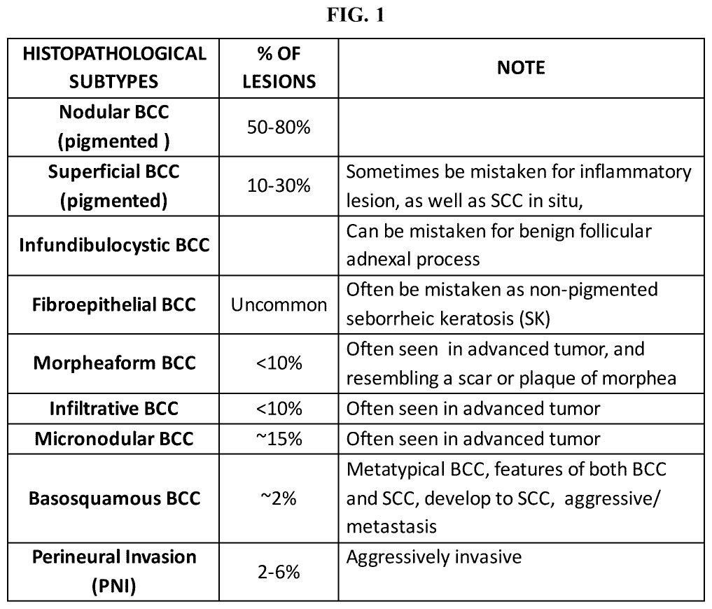

Basal cell carcinoma (BCC) is an uncontrolled growth or lesion from the basal cells (or basal keratinocytes), the deepest layer of the epidermis. In some instances, BCC is developed on sun-exposed areas, e.g., in the head and neck area. BCC is a slow-growing cancer and generally does not spread to other parts of the body. In some instances, BCC is further classified into subtypes and the subtypes comprises nodular BCC (pigmented), superficial BCC (pigmented), infundibulocystic BCC, fibroepithelial BCC, morpheaform BCC, infiltrative BCC, miconodular BCC, basosquamous BCC, and perineural invasion (PNI). Also see FIG. 1. Additional subtypes of BCC include nodulocystic, microcystic, adenoid, follicular, rodent ulcer, neurotropic, solitary basal cell carcinoma in young persons, pleomorphic, clear cell, granular cell, and singlet ring cell BCC.

Squamous cell carcinoma (SCC) (also known as cutaneous squamous cell carcinoma (CSCC) or epidermoid carcinoma) is the second most common form of skin cancer. Similar to BCC, SCC also originates from the basal keratinocytes and is a slow growing cancer, usually found in UV exposed areas such as the head and neck. In some instances, SCC is further classified into subtypes and the subtypes comprises squamous cell carcinoma in situ (also known as Bowen's disease), invasive squamous cell carcinoma (SCCI), clear cell SCC, spindle cell (sarcomatoid) SCC, SCC with single cell infiltrates, de novo SCC, verrucous carcinoma (VC), and lymphoepithelioma-like carcinoma of the skin. In some instances, the SCC subtype also comprises keratoacanthomas.

Diagnosis of skin cancers include both invasive techniques and non-invasive methods with the gold standard being biopsy followed by histopathology evaluation. In some instances, non-invasive methods have reduced specificity and/or sensitivity, and often require a biopsy step for conclusive diagnosis.

Provided herein are methods and compositions for non-invasively diagnosing or detecting a skin disease, e.g., a non-melanoma skin cancer (NMSC) or melanoma. In some instances, methods and compositions as described herein are used for diagnosing or detecting BCC. In other instances, methods and compositions as described herein are used for diagnosing or detecting SCC. In some cases, methods and compositions as described herein comprise improved sensitivity and specificity for diagnosing or detecting a skin disease, e.g., a NMSC such BCC or SCC.

### Carcinoma Assay

Methods and compositions as described herein are used for detecting gene expression levels of a gene of interest. In some instances, the gene of interest is implicated in a skin disease. In some instances, the skin disease is a non-melanoma skin cancer (NMSC). In some instances, the NMSC is BCC or SCC. In some cases, the skin disease is melanoma. Exemplary genes associated with a skin disease (e.g., a NMSC such as BCC or SCC) and, in some instances, detected using methods described herein include, but are not limited to, MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, and SPP1. In some instances, the gene of interest is MMP1. In some instances, the gene of interest is S100A7. In some instances, the gene of interest is CMPK2. In some instances, the gene of interest is IRF7. In some instances, the gene of interest is IGFL1. In some instances, the gene of interest is CXCL1. In some instances, the gene of interest is UPP1. In some instances, the gene of interest is DEFB4A. In some instances, the gene of interest is FOS. In some instances, the gene of interest is OAS3. In some instances, the gene of interest is SCD5. In some instances, the gene of interest is RTP4. In some instances, the gene of interest is VEGFA. In some instances, the gene of interest is COL5A2. In some instances, the gene of interest is IL24. In some instances, the gene of interest is AADACL2. In some instances, the gene of interest is PTCH1. In some instances, the gene of interest is CD68. In some instances, the gene of interest is PRKACA. In some instances, the gene of interest is SPP1.

In some embodiments, the gene expression levels of one or more genes of interest are detected. In some instances, at least two of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, and SPP1 are detected. In some instances, at least three of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, and SPP1 are detected. In some instances, at least four of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, and SPP1 are detected. In some instances, at least five of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, and SPP1 are detected. In some instances, at least six of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, and SPP1 are detected. In some instances, at least seven of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, and SPP1 are detected. In some instances, at least eight of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, and SPP1 are detected. In some instances, at least nine of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, and SPP1 are detected. In some instances, at least ten of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, and SPP1 are detected. In some instances, at least eleven of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, and SPP1 are detected. In some instances, at least twelve of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, and SPP1 are detected. In some instances, all of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, and SPP1 are detected.

In some embodiments, the gene expression level of one or more genes selected from IGFL1, MMP1, COL5A2, IL24, AADACL2, PTCH1, CD68, PRKACA, and SPP1 is detected. In some cases, the gene expression levels of two or more genes selected from IGFL1, MMP1, COL5A2, IL24, AADACL2, PTCH1, CD68, PRKACA, and SPP1 are detected. In some cases, the gene expression levels of three or more genes selected from IGFL1, MMP1, COL5A2, IL24, AADACL2, PTCH1, CD68, PRKACA, and SPP1 are detected. In some cases, the gene expression levels of four or more genes selected from IGFL1, MMP1, COL5A2, IL24, AADACL2, PTCH1, CD68, PRKACA, and SPP1 are detected. In some cases, the gene expression levels of five or more genes selected from IGFL1, MMP1, COL5A2, IL24, AADACL2, PTCH1, CD68, PRKACA, and SPP1 are detected. In some cases, the gene expression levels of six or more genes selected from IGFL1, MMP1, COL5A2, IL24, AADACL2, PTCH1, CD68, PRKACA, and SPP1 are detected. In some cases, the gene expression levels of seven or more genes selected from IGFL1, MMP1, COL5A2, IL24, AADACL2, PTCH1, CD68, PRKACA, and SPP1 are detected. In some cases, the gene expression levels of eight or more genes selected from IGFL1, MMP1, COL5A2, IL24, AADACL2, PTCH1, CD68, PRKACA, and SPP1 are detected. In some cases, the gene expression levels of IGFL1, MMP1, COL5A2, IL24, AADACL2, PTCH1, CD68, PRKACA, and SPP1 are detected.

In some instances, the gene expression levels of IGFL1 in combination with one or more of COL5A2, IL24, AADACL2, PTCH1, CD68, PRKACA, and SPP1 are detected. In some cases, the gene expression levels of IGFL1 in combination with two or more of COL5A2, IL24, AADACL2, PTCH1, CD68, PRKACA, and SPP1 are detected. In some cases, the gene expression levels of IGFL1 in combination with three or more of COL5A2, IL24, AADACL2, PTCH1, CD68, PRKACA, and SPP1 are detected. In some cases, the gene expression levels of IGFL1 in combination with four or more of COL5A2, IL24, AADACL2, PTCH1, CD68, PRKACA, and SPP1 are detected. In some cases, the gene expression levels of IGFL1 in combination with five or more of COL5A2, IL24, AADACL2, PTCH1, CD68, PRKACA, and SPP1 are detected. In some cases, the gene expression levels of IGFL1 in combination with six or more of COL5A2, IL24, AADACL2, PTCH1, CD68, PRKACA, and SPP1 are detected. In some cases, the gene expression levels of IGFL1 in combination with COL5A2, IL24, AADACL2, PTCH1, CD68, PRKACA, and SPP1 are detected.

In some instances, the gene expression levels of MMP1 in combination with one or more of COL5A2, IL24, AADACL2, PTCH1, CD68, PRKACA, and SPP1 are detected. In some cases, the gene expression levels of MMP1 in combination with two or more of COL5A2, IL24, AADACL2, PTCH1, CD68, PRKACA, and SPP1 are detected. In some cases, the gene expression levels of MMP1 in combination with three or more of COL5A2, IL24, AADACL2, PTCH1, CD68, PRKACA, and SPP1 are detected. In some cases, the gene expression levels of MMP1 in combination with four or more of COL5A2, IL24, AADACL2, PTCH1, CD68, PRKACA, and SPP1 are detected. In some cases, the gene expression levels of MMP1 in combination with five or more of COL5A2, IL24, AADACL2, PTCH1, CD68, PRKACA, and SPP1 are detected. In some cases, the gene expression levels of MMP1 in combination with six or more of COL5A2, IL24, AADACL2, PTCH1, CD68, PRKACA, and SPP1 are detected. In some cases, the gene expression levels of MMP1 in combination with COL5A2, IL24, AADACL2, PTCH1, CD68, PRKACA, and SPP1 are detected.

In some instances, the gene expression levels of IGFL1 and MMP1 in combination with one or more of COL5A2, IL24, AADACL2, PTCH1, CD68, PRKACA, and SPP1 are detected. In some cases, the gene expression levels of IGFL1 and MMP1 in combination with two or more of COL5A2, IL24, AADACL2, PTCH1, CD68, PRKACA, and SPP1 are detected. In some cases, the gene expression levels of IGFL1 and MMP1 in combination with three or more of COL5A2, IL24, AADACL2, PTCH1, CD68, PRKACA, and SPP1 are detected. In some cases, the gene expression levels of IGFL1 and MMP1 in combination with four or more of COL5A2, IL24, AADACL2, PTCH1, CD68, PRKACA, and SPP1 are detected. In some cases, the gene expression levels of IGFL1 and MMP1 in combination with five or more of COL5A2, IL24, AADACL2, PTCH1, CD68, PRKACA, and SPP1 are detected. In some cases, the gene expression levels of IGFL1 and MMP1 in combination with six or more of COL5A2, IL24, AADACL2, PTCH1, CD68, PRKACA, and SPP1 are detected. In some cases, the gene expression levels of IGFL1 and MMP1 in combination with COL5A2, IL24, AADACL2, PTCH1, CD68, PRKACA, and SPP1 are detected.

In some embodiments, the gene expression levels of at least two of MMP1, S100A7, CMPK2, IRF7, IGFL1, CXCL1, and UPP1 are detected. In some embodiments, the gene expression levels of at least three of MMP1, S100A7, CMPK2, IRF7, IGFL1, CXCL1, and UPP1 are detected. In some embodiments, the gene expression levels of at least four of MMP1, S100A7, CMPK2, IRF7, IGFL1, CXCL1, and UPP1 are detected. In some embodiments, the gene expression levels of at least five of MMP1, S100A7, CMPK2, IRF7, IGFL1, CXCL1, and UPP1 are detected. In some embodiments, the gene expression levels of at least six of MMP1, S100A7, CMPK2, IRF7, IGFL1, CXCL1, and UPP1 are detected. In some embodiments, the gene expression levels of all of MMP1, S100A7, CMPK2, IRF7, IGFL1, CXCL1, and UPP1 are detected.

In some embodiments, the gene expression levels of at least two of MMP1, S100A7, CMPK2, IRF7, IGFL1, and CXCL1 are detected. In some embodiments, the gene expression levels of at least three of MMP1, S100A7, CMPK2, IRF7, IGFL1, and CXCL1 are detected. In some embodiments, the gene expression levels of at least four of MMP1, S100A7, CMPK2, IRF7, IGFL1, and CXCL1 are detected. In some embodiments, the gene expression levels of at least five of MMP1, S100A7, CMPK2, IRF7, IGFL1, and CXCL1 are detected. In some embodiments, the gene expression levels of all of MMP1, S100A7, CMPK2, IRF7, IGFL1, and CXCL1 are detected.

In some embodiments, the gene expression levels of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, SPP1, or a combination thereof are used to differentiate cancer samples from non-cancer samples. In some embodiments, the gene expression levels of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, SPP1, or a combination thereof in cancer samples and non-cancer samples are analyzed using various algorithmic models. In some instances, the algorithmic model is random forest (rf) model, boosting model, logit model, lasso model, or combinations thereof. In some instances, the algorithmic model is the rf model. In some embodiments, methods for differentiating cancer samples from non-cancer samples comprises various variables including, but not limited to, interactions between MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, and SPP1. In some instances, the interactions are among at least or about 2 genes, 3 genes, 4 genes, 5 genes, 6 genes, 7 genes, 8 genes, 9 genes, 10 genes, 11 genes, 12 genes, or 13 genes. In some instances, the interactions are among at least or about 1, 2, 3, 4, 5, 6, 7, 8, 9, 10, 11, 12, 13, 14, 15, or more than 15 pairs of genes. Exemplary gene interactions are seen in Tables 1-3.

TABLE 1

| rank | rf | boosting | lasso | logit |
|---|---|---|---|---|
| 1 | MMP1.S100A7 | Genes with low | Genes with low | IGFL1.MMP1 |
| | | expression | expression | |
| 2 | CMPK2.IRF7 | CXCL1.OAS3 | IGFL1.MMP1 | CMPK2.SCD5 |
| 3 | Genes with low | IGFL1.CMPK2 | SCD5.S100A7 | UPP1v2.RTP4 |
| | expression | | | |
| 4 | IGFL1.CXCL1 | CMPK2.IRF7 | DEFB4A | UPP1v2.SCD5 |
| 5 | IGFL1.CMPK2 | MMP1.CMPK2 | UPP1v2.SCD5 | IGFL1.IRF7 |
| 6 | UPP1v2.CMPK2 | IGFL1.IRF7 | MMP1.S100A7 | IGFL1.DEFB4A |
| 7 | MMP1.CMPK2 | MMP1.S100A7 | CXCL1.IRF7 | FOS.SCD5 |
| 8 | CXCL1.IRF7 | UPP1v2.CMPK2 | MMP1.SCD5 | FOS.IRF7 |
| 9 | IGFL1.SCD5 | CXCL1.RTP4 | CMPK2 | IGFL1.RTP4 |
| 10 | IGFL1.IRF7 | CXCL1.IRF7 | CMPK2.RTP4 | RTP4 |
| 11 | CXCL1.SCD5 | MMP1.SCD5 | FOS.IRF7 | S100A7 |
| 12 | CMPK2.S100A7 | FOS.IRF7 | FOS.RTP4 | DEFB4A.FOS |
| 13 | CXCL1.S100A7 | CXCL1.SCD5 | S100A7 | SCD5 |
| 14 | IGFL1.MMP1 | CXCL1.CMPK2 | FOS.CMPK2 | UPP1v2.OAS3 |
| 15 | CXCL1.CMPK2 | FOS.CMPK2 | CMPK2.SCD5 | UPP1v2.MMP1 |

TABLE 2

| rank | rf | boosting | lasso | logit |
|---|---|---|---|---|
| 1 | SCD5.S100A7 | CXCL1.SCD5 | Genes with low | Genes with low |
| | | | expression | expression |
| 2 | CMPK2.IRF7 | CMPK2.IRF7 | UPP1v2.FOS | UPP1v2.FOS |
| 3 | CXCL1.SCD5 | MMP1.OAS3 | SCD5.VEGFA | IGFL1.VEGFA |
| 4 | MMP1.CMPK2 | SCD5.S100A7 | CXCL1.CMPK2 | FOS |
| 5 | CMPK2.S100A7 | CMPK2.S100A7 | CXCL1.SCD5 | DEFB4A.FOS |
| 6 | SCD5.VEGFA | CMPK2.SCD5 | IGFL1.SCD5 | UPP1v2.CMPK2 |
| 7 | IGFL1.CMPK2 | Genes with low | CMPK2.VEGFA | IGFL1.SCD5 |
| | | expression | | |
| 8 | MMP1.S100A7 | FOS.SCD5 | IGFL1.RTP4 | IGFL1.DEFB4A |
| 9 | CMPK2.SCD5 | MMP1.CMPK2 | FOS.IRF7 | IGFL1.IRF7 |
| 10 | CXCL1.IRF7 | CXCL1.OAS3 | DEFB4A | UPP1v2.IRF7 |
| 11 | CMPK2.VEGFA | IGFL1.CMPK2 | DEFB4A.FOS | UPP1v2.SCD5 |
| 12 | CXCL1.OAS3 | SCD5.VEGFA | CMPK2.OAS3 | UPP1v2 |
| 13 | MMP1.SCD5 | MMP1.S100A7 | DEFB4A.RTP4 | UPP1v2.CXCL1 |
| 14 | CXCL1.CMPK2 | MMP1.SCD5 | CXCL1.OAS3 | IGFL1.RTP4 |
| 15 | MMP1.OAS3 | CXCL1.CMPK2 | UPP1v2.IRF7 | UPP1v2.DEFB4A |

TABLE 3

| Rank | rf | boosting | logit | lasso |
|---|---|---|---|---|
| 1 | IGFL1.COL5A2 | IL24.AADACL2 | SPP1 | PRKACA.SPP1 |
| 2 | IL24.AADACL2 | IGFL1.COL5A2 | COL5A2.AADACL2 | IGFL1.AADACL2 |
| 3 | PTCH1.CD68 | PRKACA.SPP1 | CD68.PRKACA | COL5A2 |
| 4 | PRKACA.SPP1 | AADACL2.MMP1 | PTCH1.MMP1 | IGFL1.SPP1 |
| 5 | AADACL2.MMP1 | AADACL2.SPP1 | IGFL1 | CD68.MMP1 |
| 6 | IGFL1.IL24 | PTCH1.CD68 | IGFL1.CD68 | COL5A2.CD68 |
| 7 | AADACL2.SPP1 | PRKACA.MMP1 | IGFL1.PTCH1 | PTCH1.SPP1 |
| 8 | IGFL1 | IGFL1.IL24 | IL24 | COL5A2.AADACL2 |
| 9 | PRKACA.MMP1 | IGFL1.PRKACA | AADACL2.CD68 | SPP1.MMP1 |
| 10 | IGFL1.PRKACA | AADACL2.CD68 | PTCH1.PRKACA | IGFL1.MMP1 |
| 11 | IGFL1.SPP1 | IL24.PRKACA | IGFL1.PRKACA | COL5A2.SPP1 |
| 12 | AADACL2.CD68 | SPP1.MMP1 | COL5A2.SPP1 | PTCH1.MMP1 |
| 13 | IL24.PRKACA | CD68.PRKACA | PTCH1 | IL24.CD68 |
| 14 | IGFL1.PTCH1 | COL5A2.AADACL2 | AADACL2.MMP1 | IGFL1.COL5A2 |
| 15 | IGFL1.AADACL2 | COL5A2.CD68 | PRKACA | IGFL1.CD68 |

In some embodiments, methods for differentiating cancer samples from non-cancer samples comprise detecting the gene expression levels of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, SPP1, or a combination thereof in cancer samples and non-cancer samples. In some instances, the cancer samples comprise basal cell carcinoma (BCC), squamous cell carcinoma (SCC), or a combination thereof. In some instances, the cancer samples comprise BCC and SCC. In some instances, the non-cancer samples comprise actinic keratosis (AK), seborrheic keratosis (SK), normal samples, or a combination thereof. In some instances, the non-cancer samples comprise actinic keratosis (AK), seborrheic keratosis (SK), and normal samples.

In some embodiments, the gene expression levels of IGFL1, MMP1, COL5A2, IL24, PTCH1, PRKACA, AADACL2, CD68, or SPP1 are upregulated in a skin disease, e.g., a non-melanoma skin cancer sample (optionally, BCC and/or SCC sample). In some instances, an upregulated gene expression level of IGFL1, MMP1, COL5A2, IL24, PTCH1, PRKACA, AADACL2, CD68, or SPP1, or a combination thereof are used to distinguish between a cancer sample from a non-cancer sample. For example, an upregulated gene expression level of IGFL1, MMP1, COL5A2, IL24, PTCH1, PRKACA, AADACL2, CD68, or SPP1, or a combination thereof are used to distinguish between BCC and/or SCC samples from AK, SK, normal sample or a combination thereof.

In some instances, BCC and SCC samples are compared to AK, SK, and normal samples. In some instances, BCC and SCC samples are compared to AK and SK samples. In some instances, BCC and SCC samples are compared to AK and normal samples. In some instances, BCC and SCC samples are compared SK and normal samples. In some instances, BCC and SCC samples are compared to AK samples. In some instances, BCC and SCC samples are compared to SK samples. In some instances, BCC and SCC samples are compared to normal samples. In some instances, BCC samples are compared to AK, SK, and normal samples. In some instances, BCC samples are compared to AK and normal samples. In some instances, BCC samples are compared to AK and SK samples. In some instances, BCC samples are compared to SK and normal samples. In some instances, BCC samples are compared to AK samples. In some instances, BCC samples are compared to SK samples. In some instances, BCC samples are compared to normal samples. In some instances, SCC samples are compared to AK, SK, and normal samples. In some instances, SCC samples are compared to AK and normal samples. In some instances, SCC samples are compared to AK and SK samples. In some instances, SCC samples are compared to SK and normal samples. In some instances, SCC samples are compared to AK samples. In some instances, SCC samples are compared to SK samples. In some instances, SCC samples are compared to normal samples. In some instances, BCC samples are compared to SCC samples.

In some embodiments, methods for differentiating cancer samples from non-cancer samples comprising detecting the gene expression levels of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, SPP1, or a combination thereof comprise improved specificity and sensitivity. In some embodiments, the specificity is at least or about 70%, 75%, 80%, 85%, 90%, or more than 95% when detecting the gene expression levels of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, SPP1, or a combination thereof. In some embodiments, the specificity is at least or about 70%, 75%, 80%, 85%, 90%, or more than 95% when detecting the gene expression levels of IGFL1, MMP1, COL5A2, IL24, AADACL2, PTCH1, CD68, PRKACA, SPP1, or a combination thereof. In some embodiments, the specificity is at least or about 70%, 75%, 80%, 85%, 90%, or more than 95% when detecting the gene expression levels of MMP1, S100A7, CMPK2, IRF7, IGFL1, CXCL1, UPP1, or a combination thereof. In some embodiments, the specificity is at least or about 70%, 75%, 80%, 85%, 90%, or more than 95% when detecting the gene expression levels of MMP1, S100A7, CMPK2, IRF7, IGFL1, CXCL1, or a combination thereof.

In some embodiments, the sensitivity is at least or about 70%, 75%, 80%, 85%, 90%, or more than 95% when detecting the gene expression levels of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, SPP1, or a combination thereof. In some embodiments, the sensitivity is at least or about 70%, 75%, 80%, 85%, 90%, or more than 95% when detecting the gene expression levels of IGFL1, MMP1, COL5A2, IL24, AADACL2, PTCH1, CD68, PRKACA, SPP1, or a combination thereof. In some embodiments, the sensitivity is at least or about 70%, 75%, 80%, 85%, 90%, or more than 95% when detecting the gene expression levels of MMP1, S100A7, CMPK2, IRF7, IGFL1, CXCL1, UPP1, or a combination thereof. In some embodiments, the sensitivity is at least or about 70%, 75%, 80%, 85%, 90%, or more than 95% when detecting the gene expression levels of MMP1, S100A7, CMPK2, IRF7, IGFL1, CXCL1, or a combination thereof.

In some embodiments, cancer samples are differentiated from non-cancer samples when amplification of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, SPP1, or a combination thereof is detected. In some embodiments, amplification of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, SPP1, or a combination thereof is not detected in non-cancer samples.

Various methods for detecting gene expression levels are contemplated herein. For example, a set of probes are used to detect at least two of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, and SPP1. In some cases, no more than 3, 4, 5, 6, 7, 8, 9, 10, 15, 20, or 22 genes are detected. In some instances, a set of probes are used to detect at least three of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, and SPP1. In some instances, a set of probes are used to detect at least four of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, and SPP1. In some instances, a set of probes are used to detect at least five of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, and SPP1. In some instances, a set of probes are used to detect at least six of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, and SPP1. In some instances, a set of probes are used to detect at least seven of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, and SPP1. In some instances, a set of probes are used to detect at least eight of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, and SPP1. In some instances, a set of probes are used to detect at least nine of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, and SPP1. In some instances, a set of probes are used to detect at least ten of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, and SPP1. In some instances, a set of probes are used to detect at least eleven of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, and SPP1. In some instances, a set of probes are used to detect at least twelve of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, and SPP1. In some instances, a set of probes are used to detect all of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, and SPP1.

In some embodiments, the set of probes are used to detect at least the gene expression levels of at least two of IGFL1, MMP1, COL5A2, IL24, AADACL2, PTCH1, CD68, PRKACA, and SPP1. In some cases, no more than 3, 4, 5, 6, 7, 8, or 9 genes are detected. In some cases, the set of probes are used to detect at least the gene expression levels of at least three of IGFL1, MMP1, COL5A2, IL24, AADACL2, PTCH1, CD68, PRKACA, and SPP1. In some cases, the set of probes are used to detect at least the gene expression levels of at least four of IGFL1, MMP1, COL5A2, IL24, AADACL2, PTCH1, CD68, PRKACA, and SPP1. In some cases, the set of probes are used to detect at least the gene expression levels of at least five of IGFL1, MMP1, COL5A2, IL24, AADACL2, PTCH1, CD68, PRKACA, and SPP1. In some cases, the set of probes are used to detect at least the gene expression levels of at least six of IGFL1, MMP1, COL5A2, IL24, AADACL2, PTCH1, CD68, PRKACA, and SPP1. In some cases, the set of probes are used to detect at least the gene expression levels of IGFL1, MMP1, COL5A2, IL24, AADACL2, PTCH1, CD68, PRKACA, and SPP1.

In some embodiments, the set of probes are used to detect at least the gene expression levels of at least two of MMP1, S100A7, CMPK2, IRF7, IGFL1, CXCL1, and UPP1. In some embodiments, the set of probes are used to detect at least three of MMP1, S100A7, CMPK2, IRF7, IGFL1, CXCL1, and UPP1. In some embodiments, the set of probes are used to detect at least four of MMP1, S100A7, CMPK2, IRF7, IGFL1, CXCL1, and UPP1. In some embodiments, the set of probes are used to detect at least five of MMP1, S100A7, CMPK2, IRF7, IGFL1, CXCL1, and UPP1. In some embodiments, the set of probes are used to detect at least six of MMP1, S100A7, CMPK2, IRF7, IGFL1, CXCL1, and UPP1. In some embodiments, the set of probes are used to detect all of MMP1, S100A7, CMPK2, IRF7, IGFL1, CXCL1, and UPP1.

In some embodiments, the set of probes are used to detect the gene expression levels of at least two of MMP1, S100A7, CMPK2, IRF7, IGFL1, and CXCL1. In some embodiments, the set of probes are used to detect at least three of MMP1, S100A7, CMPK2, IRF7, IGFL1, and CXCL1. In some embodiments, the set of probes are used to detect at least four of MMP1, S100A7, CMPK2, IRF7, IGFL1, and CXCL1. In some embodiments, the set of probes are used to detect at least five of MMP1, S100A7, CMPK2, IRF7, IGFL1, and CXCL1. In some embodiments, the set of probes are used to detect of all of MMP1, S100A7, CMPK2, IRF7, IGFL1, and CXCL1.

In some embodiments, the set of probes comprises polynucleotides. In some instances, the set of probes comprises polynucleotides for two different exons of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, or SPP1. In some instances, the gene expression levels are detected following hybridization of the set of probes to at least two of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, and SPP1. In some embodiments, the set of probes detects RNA. In some embodiments, the set of probes detects mRNA. In some embodiments, the set of probes detects DNA.

Probes for detecting gene expression levels of at least two of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, or SPP1, in certain embodiments, are used for an amplification reaction. In some embodiments, the amplification reaction is PCR. In some embodiments, the amplification reaction is quantitative such as qPCR. In some embodiments, the PCR reaction utilizes a TaqMan™ or a similar quantitative PCR technology.

In some embodiments, a number of probes in the set of probes is at least or about 1, 2, 3, 5, 6, 7, 8, 9, 10, 11, 12, 13, 14, 15, 16, 17, 18, 19, 20, 25, 30, or more than 30 probes. In some embodiments, the number of probes in the set of probes is about 6 probes. In some embodiments, the number of probes in the set of probes is about 7 probes. In some embodiments, the number of probes in the set of probes is about 8 probes. In some embodiments, the number of probes in the set of probes is about 9 probes. In some embodiments, the number of probes in the set of probes is about 13 probes.

In some embodiments, the set of probes comprises one or more primer pairs. In some embodiments, a number of primer pairs is at least or about 1, 2, 3, 5, 6, 7, 8, 9, 10, 11, 12, 13, 14, 15, 16, 17, 18, 19, 20, 25, 30, or more than 30 primer pairs. In some embodiments, the number of primer pairs is about 6 primer pairs. In some embodiments, the number of primer pairs is about 7 primer pairs. In some embodiments, the number of primer pairs is about 13 primer pairs.

In some embodiments, one or more probes in the set of probes is labeled. In some embodiments, the one or more probe is labeled with a radioactive label, a fluorescent label, an enzyme, a chemiluminescent tag, a colorimetric tag, an affinity tag or other labels or tags that are known in the art.

Exemplary affinity tags include, but are not limited to, biotin, desthiobiotin, histidine, polyhistidine, myc, hemagglutinin (HA), FLAG, glutathione S transferase (GST), or derivatives thereof. In some embodiments, the affinity tag is recognized by avidin, streptavidin, nickel, or glutathione.

In some embodiments, the fluorescent label is a fluorophore, a fluorescent protein, a fluorescent peptide, quantum dots, a fluorescent dye, a fluorescent material, or variations or combinations thereof.

Exemplary fluorophores include, but are not limited to, Alexa-Fluor dyes (e. g., Alexa Fluor®350, Alexa Fluor® 405, Alexa Fluor® 430, Alexa Fluor® 488, Alexa Fluor® 500, Alexa Fluor® 514, Alexa Fluor® 532, Alexa Fluor® 546, Alexa Fluor® 555, Alexa Fluor® 568, Alexa Fluor® 594, Alexa Fluor® 610, Alexa Fluor® 633, Alexa Fluor® 647, Alexa Fluor® 660, Alexa Fluor® 680, Alexa Fluor® 700, and Alexa Fluor® 750), APC, Cascade Blue, Cascade Yellow and R-phycoerythrin (PE), DyLight 405, DyLight 488, DyLight 550, DyLight 650, DyLight 680, DyLight 755, DyLight 800, FITC, Pacific Blue, PerCP, Rhodamine, and Texas Red, Cy5, Cy5.5, Cy7.

Examples of fluorescent peptides include GFP (Green Fluorescent Protein) or derivatives of GFP (e.g., EBFP, EBFP2, Azurite, mKalama1, ECFP, Cerulean, CyPet, YFP, Citrine, Venus, and YPet.

Examples of fluorescent dyes include, but are not limited to, xanthenes (e.g., rhodamines, rhodols and fluoresceins, and their derivatives); bimanes; coumarins and their derivatives (e.g., umbelliferone and aminomethyl coumarins); aromatic amines (e.g., dansyl; squarate dyes); benzofurans; fluorescent cyanines; indocarbocyanines; carbazoles; dicyanomethylene pyranes; polymethine; oxabenzanthrane; xanthene; pyrylium; carbostyl; perylene; acridone; quinacridone; rubrene; anthracene; coronene; phenanthrecene; pyrene; butadiene; stilbene; porphyrin; pthalocyanine; lanthanide metal chelate complexes; rare-earth metal chelate complexes; and derivatives of such dyes. In some embodiments, the fluorescein dye is, but not limited to, 5-carboxyfluorescein, fluorescein-5-isothiocyanate, fluorescein-6-isothiocyanate and 6-carboxyfluorescein. In some embodiments, the rhodamine dye is, but not limited to, tetramethylrhodamine-6-isothiocyanate, 5-carboxytetramethylrhodamine, 5-carboxy rhodol derivatives, tetramethyl and tetraethyl rhodamine, diphenyldimethyl and diphenyldiethyl rhodamine, dinaphthyl rhodamine, and rhodamine 101 sulfonyl chloride (sold under the tradename of TEXAS RED®). In some embodiments, the cyanine dye is Cy3, Cy3B, Cy3.5, Cy5, Cy5.5, Cy7, IRDYE680, Alexa Fluor 750, IRDye800CW, or ICG.

In some embodiments, the gene expression levels of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, SPP1, or a combination thereof is measured using PCR. Examples of PCR techniques include, but are not limited to quantitative PCR (qPCR), single cell PCR, PCR-RFLP, digital PCR (dPCR), droplet digital PCR (ddPCR), single marker qPCR, hot start PCR, and Nested PCR.

In some embodiments, the expression levels are measured using qPCR. In some embodiments, the qPCR comprises use of fluorescent dyes or fluorescent probes. In some embodiments, the fluorescent dye is an intercalating dye. Examples of intercalating dyes include, but are not limited to, intercalating dyes include SYBR green I, SYBR green II, SYBR gold, ethidium bromide, methylene blue, Pyronin Y, DAPI, acridine orange, Blue View, or phycoerythrin. In some embodiments, the qPCR comprises use of more than one fluorescent probe. In some embodiments, the use of more than one fluorescent probes allows for multiplexing. For example, different non-classical variants are hybridized to different fluorescent probes and can be detected in a single qPCR reaction.

Methods and compositions described herein, in some embodiments, further comprise detecting a mutational change in a gene of interest. In some instances, the mutational change is detected in TERT, CDKN2A, TP53, PTCH1, or a combination thereof. In some instances, the mutational change is detected in TERT. In some instances, the mutational change is detected in CDKN2A. In some instances, the mutational change is detected in TP53. In some instances, the mutational change is detected in PTCH1. Exemplary amino acid sequences for TERT, CDKN2A, TP53, and PTCH1 are illustrated in Table 4.

TABLE 4

| SEQ | | Accession | |
|---|---|---|---|
| ID NO | Gene Name | Number | Amino Acid Sequence |
| 1 | TP53 | P04637 | MEEPQSDPSVEPPLSQETFSDLWKLLPENNVLSPLPSQA |
| | | | MDDLMLSPDDIEQWFTEDPGPDEAPRMPEAAPPVAPAPA |
| | | | APTPAAPAPAPSWPLSSSVPSQKTYQGSYGFRLGFLHSG |
| | | | TAKSVTCTYSPALNKMFCQLAKTCPVQLWVDSTPPPGTR |
| | | | VRAMAIYKQSQHMTEVVRRCPHHERCSDSDGLAPPQHLI |
| | | | RVEGNLRVEYLDDRNTFRHSVVVPYEPPEVGSDCTTIHY |
| | | | NYMCNSSCMGGMNRRPILTIITLEDSSGNLLGRNSFEVR |
| | | | VCACPGRDRRTEEENLRKKGEPHHELPPGSTKRALPNNT |
| | | | SSSPQPKKKPLDGEYFTLQIRGRERFEMFRELNEALELK |
| | | | DAQAGKEPGGSRAHSSHLKSKKGQSTSRHKKLMFKTEGP |
| | | | DSD |
| 2 | TERT | O14746 | MPRAPRCRAVRSLLRSHYREVLPLATFVRRLGPQGWRLV |
| | | | QRGDPAAFRALVAQCLVCVPWDARPPPAAPSFRQVSCLK |
| | | | ELVARVLQRLCERGAKNVLAFGFALLDGARGGPPEAFTT |
| | | | SVRSYLPNTVTDALRGSGAWGLLLRRVGDDVLVHLLARC |
| | | | ALFVLVAPSCAYQVCGPPLYQLGAATQARPPPHASGPRR |
| | | | RLGCERAWNHSVREAGVPLGLPAPGARRRGGSASRSLPL |
| | | | PKRPRRGAAPEPERTPVGQGSWAHPGRTRGPSDRGFCVV |
| | | | SPARPAEEATSLEGALSGTRHSHPSVGRQHHAGPPSTSR |
| | | | PPRPWDTPCPPVYAETKHFLYSSGDKEQLRPSFLLSSLR |
| | | | PSLTGARRLVETIFLGSRPWMPGTPRRLPRLPQRYWQMR |
| | | | PLFLELLGNHAQCPYGVLLKTHCPLRAAVTPAAGVCARE |
| | | | KPQGSVAAPEEEDTDPRRLVQLLRQHSSPWQVYGFVRAC |
| | | | LRRLVPPGLWGSRHNERRFLRNTKKFISLGKHAKLSLQE |
| | | | LTWKMSVRDCAWLRRSPGVGCVPAAEHRLREEILAKFLH |
| | | | WLMSVYVVELLRSFFYVTETTFQKNRLFFYRKSVWSKLQ |
| | | | SIGIRQHLKRVQLRELSEAEVRQHREARPALLTSRLRFI |
| | | | PKPDGLRPIVNMDYVVGARTFRREKRAERLTSRVKALFS |
| | | | VLNYERARRPGLLGASVLGLDDIHRAWRTFVLRVRAQDP |
| | | | PPELYFVKVDVTGAYDTIPQDRLTEVIASIIKPQNTYCV |
| | | | RRYAVVQKAAHGHVRKAFKSHVSTLTDLQPYMRQFVAHL |
| | | | QETSPLRDAVVIEQSSSLNEASSGLFDVFLRFMCHHAVR |
| | | | IRGKSYVQCQGIPQGSILSTLLCSLCYGDMENKLFAGIR |
| | | | RDGLLLRLVDDFLLVTPHLTHAKTFLRTLVRGVPEYGCV |
| | | | VNLRKTVVNFPVEDEALGGTAFVQMPAHGLFPWCGLLLD |
| | | | TRTLEVQSDYSSYARTSIRASLTFNRGFKAGRNMRRKLF |
| | | | GVLRLKCHSLFLDLQVNSLQTVCTNIYKILLLQAYRFHA |
| | | | CVLQLPFHQQVWKNPTFFLRVISDTASLCYSILKAKNAG |
| | | | MSLGAKGAAGPLPSEAVQWLCHQAFLLKLTRHRVTYVPL |
| | | | LGSLRTAQTQLSRKLPGTTLTALEAAANPALPSDFKTIL |
| | | | D |
| 3 | PTCH1 | Q13635 | MASAGNAAEPQDRGGGGSGCIGAPGRPAGGGRRRRTGGL |
| | (isoform L) | | RRAAAPDRDYLHRPSYCDAAFALEQISKGKATGRKAPLW |
| | | | LRAKFQRLLFKLGCYIQKNCGKFLVVGLLIFGAFAVGLK |
| | | | AANLETNVEELWVEVGGRVSRELNYTRQKIGEEAMFNPQ |
| | | | LMIQTPKEEGANVLTTEALLQHLDSALQASRVHVYMYNR |
| | | | QWKLEHLCYKSGELITETGYMDQIIEYLYPCLIITPLDC |
| | | | FWEGAKLQSGTAYLLGKPPLRWTNFDPLEFLEELKKINY |
| | | | QVDSWEEMLNKAEVGHGYMDRPCLNPADPDCPATAPNKN |
| | | | STKPLDMALVLNGGCHGLSRKYMHWQEELIVGGTVKNST |
| | | | GKLVSAHALQTMFQLMTPKQMYEHFKGYEYVSHINWNED |
| | | | KAAAILEAWQRTYVEVVHQSVAQNSTQKVLSFTTTTLDD |
| | | | ILKSFSDVSVIRVASGYLLMLAYACLTMLRWDCSKSQGA |
| | | | VGLAGVLLVALSVAAGLGLCSLIGISFNAATTQVLPFLA |
| | | | LGVGVDDVFLLAHAFSETGQNKRIPFEDRTGECLKRTGA |
| | | | SVALTSISNVTAFFMAALIPIPALRAFSLQAAVVVVFNF |
| | | | AMVLLIFPAILSMDLYRREDRRLDIFCCFTSPCVSRVIQ |
| | | | VEPQAYTDTHDNTRYSPPPPYSSHSFAHETQITMQSTVQ |
| | | | LRTEYDPHTHVYYTTAEPRSEISVQPVTVTQDTLSCQSP |
| | | | ESTSSTRDLLSQFSDSSLHCLEPPCTKWTLSSFAEKHYA |
| | | | PFLLKPKAKVVVIFLFLGLLGVSLYGTTRVRDGLDLTDI |
| | | | VPRETREYDFIAAQFKYFSFYNMYIVTQKADYPNIQHLL |
| | | | YDLHRSFSNVKYVMLEENKQLPKMWLHYFRDWLQGLQDA |
| | | | FDSDWETGKIMPNNYKNGSDDGVLAYKLLVQTGSRDKPI |
| | | | DISQLTKQRLVDADGIINPSAFYIYLTAWVSNDPVAYAA |
| | | | SQANIRPHRPEWVHDKADYMPETRLRIPAAEPIEYAQFP |
| | | | FYLNGLRDTSDFVEAIEKVRTICSNYTSLGLSSYPNGYP |
| | | | FLFWEQYIGLRHWLLLFISVVLACTFLVCAVFLLNPWTA |
| | | | GIIVMVLALMTVELFGMMGLIGIKLSAVPVVILIASVGI |
| | | | GVEFTVHVALAFLTAIGDKNRRAVLALEHMFAPVLDGAV |
| | | | STLLGVLMLAGSEFDFIVRYFFAVLAILTILGVLNGLVL |
| | | | LPVLLSFFGPYPEVSPANGLNRLPTPSPEPPPSVVRFAM |
| | | | PPGHTHSGSDSSDSEYSSQTTVSGLSEELRHYEAQQGAG |
| | | | GPAHQVIVEATENPVFAHSTVVHPESRHHPPSNPRQQPH |
| | | | LDSGSLPPGRQGQQPRRDPPREGLWPPPYRPRRDAFEIS |
| | | | TEGHSGPSNRARWGPRGARSHNPRNPASTAMGSSVPGYC |
| | | | QPITTVTASASVTVAVHPPPVPGPGRNPRGGLCPGYPET |
| | | | DHGLFEDPHVPFHVRCERRDSKVEVIELQDVECEERPRG |
| | | | SSSN |
| 4 | PTCH1 | NP_001077074.1 | MFNPQLMIQTPKEEGANVLTTEALLQHLDSALQASRVHV |
| | (isoform S) | | YMYNRQWKLEHLCYKSGELITETGYMDQIIEYLYPCLII |
| | | | TPLDCFWEGAKLQSGTAYLLGKPPLRWTNFDPLEFLEEL |
| | | | KKINYQVDSWEEMLNKAEVGHGYMDRPCLNPADPDCPAT |
| | | | APNKNSTKPLDMALVLNGGCHGLSRKYMHWQEELIVGGT |
| | | | VKNSTGKLVSAHALQTMFQLMTPKQMYEHFKGYEYVSHI |
| | | | NWNEDKAAAILEAWQRTYVEVVHQSVAQNSTQKVLSFTT |
| | | | TTLDDILKSFSDVSVIRVASGYLLMLAYACLTMLRWDCS |
| | | | KSQGAVGLAGVLLVALSVAAGLGLCSLIGISFNAATTQV |
| | | | LPFLALGVGVDDVFLLAHAFSETGQNKRIPFEDRTGECL |
| | | | KRTGASVALTSISNVTAFFMAALIPIPALRAFSLQAAVV |
| | | | VVFNFAMVLLIFPAILSMDLYRREDRRLDIFCCFTSPCV |
| | | | SRVIQVEPQAYTDTHDNTRYSPPPPYSSHSFAHETQITM |
| | | | QSTVQLRTEYDPHTHVYYTTAEPRSEISVQPVTVTQDTL |
| | | | SCQSPESTSSTRDLLSQFSDSSLHCLEPPCTKWTLSSFA |
| | | | EKHYAPFLLKPKAKVVVIFLFLGLLGVSLYGTTRVRDGL |
| | | | DLTDIVPRETREYDFIAAQFKYFSFYNMYIVTQKADYPN |
| | | | IQHLLYDLHRSFSNVKYVMLEENKQLPKMWLHYFRDWLQ |
| | | | GLQDAFDSDWETGKIMPNNYKNGSDDGVLAYKLLVQTGS |
| | | | RDKPIDISQLTKQRLVDADGIINPSAFYIYLTAWVSNDP |
| | | | VAYAASQANIRPHRPEWVHDKADYMPETRLRIPAAEPIE |
| | | | YAQFPFYLNGLRDTSDFVEAIEKVRTICSNYTSLGLSSY |
| | | | PNGYPFLFWEQYIGLRHWLLLFISVVLACTFLVCAVFLL |
| | | | NPWTAGIIVMVLALMTVELFGMMGLIGIKLSAVPVVILI |
| | | | ASVGIGVEFTVHVALAFLTAIGDKNRRAVLALEHMFAPV |
| | | | LDGAVSTLLGVLMLAGSEFDFIVRYFFAVLAILTILGVL |
| | | | NGLVLLPVLLSFFGPYPEVSPANGLNRLPTPSPEPPPSV |
| | | | VRFAMPPGHTHSGSDSSDSEYSSQTTVSGLSEELRHYEA |
| | | | QQGAGGPAHQVIVEATENPVFAHSTVVHPESRHHPPSNP |
| | | | RQQPHLDSGSLPPGRQGQQPRRDPPREGLWPPPYRPRRD |
| | | | AFEISTEGHSGPSNRARWGPRGARSHNPRNPASTAMGSS |
| | | | VPGYCQPITTVTASASVTVAVHPPPVPGPGRNPRGGLCP |
| | | | GYPETDHGLFEDPHVPFHVRCERRDSKVEVIELQDVECE |
| | | | ERPRGSSSN |
| 5 | CDKN2A | P42771 | MEPAAGSSMEPSADWLATAAARGRVEEVRALLEAGALPN |
| | | | APNSYGRRPIQVMMMGSARVAELLLLHGAEPNCADPATL |
| | | | TRPVHDAAREGFLDTLVVLHRAGARLDVRDAWGRLPVDL |
| | | | AEELGHRDVARYLRAAAGGTRGSNHARIDAAEGPSDIPD |

TERT, also known as Telomerase Reverse Transcriptase or Telomerase-Associated Protein 2, encodes the TERT protein. The TERT protein is the catalytic subunit of the protein telomerase. In some instances, a mutation in TERT is correlated with a non-melanoma skin cancer (e.g., BCC and/or SCC). In some instances, a mutation is in the TERT promoter. In some instances, a mutation is at least or about 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, or more than 200 base pairs upstream of the translation start site of the TERT promoter. In some instances, one or more mutations are in the TERT promoter. In some instances, the one or more mutations in the TERT promoter are a G to A mutation. In some instances, the one or more mutations in the TERT promoter are a T to G mutation. In some instances, the one or more mutations in the TERT promoter are a C to T mutation. In some instances, one or more mutations in the TERT promoter result in increased expression of TERT. In some instances, one or more mutations in the TERT promoter result in increased expression or activity of TERT protein.

Exemplary mutations in TERT include, but not limited to, 1,295,228 C>T (C228T) and 1,295,250 C>T (C250T). In some instances, C228T is a mutation corresponding to −124 C>T from the translation start site in the TERT promoter. In some instances, C250T is a mutation corresponding to −146 C>T from the translation start site in the TERT promoter. In some instances, a mutation is a nucleotide sequence of TERT. For example, the mutation in the nucleotide sequence includes, but not limited to, 571A>G, 648G>T, 1127C>T, 1135T>C, 1216G>A, 1217G>A, 1281C>T, 1284G>A, 1405C>T, 1461C>T, 1529G>A, 1541T>A, 1566G>A, 1689C>T, 1695G>A, 1782G>A, 1831G>A, 1841C>T, 1882G>A, 1928G>A, 2009C>A, 2067C>T, 2152G>A, 2162C>G, 2163C>T, 2178G>A, 2208G>A, 2254C>A, 2262C>G, 2271G>A, 2272G>A, 2283C>T, 2328C>T, 2361G>A, 2391C>T, 2405G>A, 2436C>T, 2456G>A, 2472C>T, 2499G>A, 2508C>A, 2568G>A, 2589C>T, 2633C>T, 2640G>A, 2725G>A, 2750C>T, 2755T>A, 2758G>A, 2773C>T, 2784C>T, 2786C>T, 2896G>A, 3015C>T, 3057C>T, 3084C>A, 3096C>T, 3097C>T, 3139C>T, 3198C>T, 3200C>T, 3284C>G, 3345G>A, 3363G>A, 1-100C>T, 1-101C>T, 1-101_1-100CC>TT, 1-106_1-105CC>TT, 1-111C>T, 1-124C>A, 1-124C>T, 1-125C>T, 1-125_1-124CC>TT, 1-126C>T, 1-126_1-124CCC>TTT, 1-126_1-125CC>TT, 1-127_1-126CC>TT, 1-139_1-138CC>TT, 1-144C>T, 1-145C>T, 1-146C>T, 1-149C>T, 1-150C>T, 1-154C>T, 1-156C>T, 1-156C>T, 1-159C>T, 1-176C>T, 1-187C>T, 1-242C>T, 1-46C>T, 1-57A>C, 1-58C>T, 1-90_1-89GC>TT, and 1-91C>T.

In some instances, a mutation in TERT is in exon 1, 2, 3, 4, 5, 6, 7, 8, 9, 10, 11, 12, 13, 14, 15, 16, or a combination thereof of TERT. In some instances, a mutation in TERT is a mutation in a peptide sequence. In some instances, the mutation results in a missense substitution, a nonsense substitution (*), a coding silent substitution, deletion (del), an insertion (ins), or a frameshift (fs). Exemplary mutations include, but are not limited to, S191G, L216L, P376L, L379L, G406R, G406E, A427A, R428R, L469L, F487F, W510*, V514E, R522R, V563V, E565E, K594K, E611K, P614L, D628N, R643K, A670E, A689A, D718N, P721R, P721P, T726T, Q736Q, H752N, H754Q, K757K, A758T, S761S, F776F, R787R, S797S, S802N, F812F, R819H, S824S, Q833Q, I836I, G856G, L863L, T878I, A880A, V909I, T917M, F919I, V920I, H925Y, F928F, P929L, A966T, L1005L, L1019L, N1028K, F1032F, L1033L, A1040T, L1047L, P1066P, S1067F, S1095*, L1115L, and P1121P. In some cases, the mutation(s) are at the corresponding residue positions as set forth in SEQ ID NO: 2.

The gene CDKN2A, also known as cyclin-dependent kinase inhibitor 2A, encodes two proteins p16^{INK4a} and p14^{ARF}. p16^{INK4a} is transcribed from exon 1α and p14^{ARF} is transcribed from exon 1β and both genes are involved in cellular senescence. In some instances, a mutation in CDKN2A is correlated with anon-melanoma skin cancer (e.g., BCC and/or SCC). In some instances, a mutation is a nucleotide sequence of CDKN2A. For example, the mutation in the nucleotide sequence includes, but not limited to, 1_471del471, 4G>A, 9_32del24, 10G>T, 12G>A, 42C>G, 44G>A, 45G>A, 47_50delTGGC, 58G>C, 66_67GG>AA, 68delG, 83T>G, 92T>C, 95_112del18, 97G>T, 104G>A, 104G>T, 106delG, 107C>G, 109C>T, 113C>T, 128_129delGT, 132delC, 142_143CC>TT, 143C>T, 144G>A, 147_148CC>AT, 147_148CC>TT, 148C>T, 151_457del307, 158_159delTG, 161T>A, 163G>C, 164G>A, 166_167insA, 168_169insG, 169_170GC>TT, 170C>T, 170_172CCC>TIT, 171C>T, 171_172CC>TT, 171_178delCCGAGTGG, 172C>T, 172_173insC, 172_179delCGAGTGGC, 172delC, 176T>G, 179C>T, 179_180insC, 181G>T, 181_202>AC, 185T>C, 188T>C, 192G>A, 194T>C, 196_198CAC>TAG, 198_199insA, 199G>A, 202G>A, 203_204delCG, 203delC, 204G>A, 205G>T, 209C>T, 212A>T, 215G>A, 218C>T, 219C>T, 222C>A, 223C>T, 223_224CC>T, 225_243del19, 227C>T, 236C>T, 237C>T, 237_238CC>TT, 238C>T, 238_247del10, 239G>A, 241C>T, 242C>A, 242C>T, 242_243CC>TT, 243_244ins19, 243_244insC, 244G>A, 245T>A, 245T>C, 247C>G, 247C>T, 248A>G, 248A>T, 248_249AC>CT, 249C>G, 250G>A, 250_270del21, 254C>T, 257_259delCCC, 259C>T, 259delC, 260G>A, 261_262GG>AA, 262G>A, 262G>T, 264_265GG>AA, 266delG, 268T>C, 270C>T, 286_294del9, 290T>C, 290T>G, 290_294delTGCAC, 295C>T, 297G>A, 297_298GG>AC, 299C>T, 301G>T, 304G>A, 305C>T, 311T>G, 319C>T, 320G>A, 329G>A, 329_330GG>AA, 330G>A, 331G>A, 334C>G, 335G>C, 341C>A, 341C>T, 341_342CC>TT, 342C>T, 346G>T, 346_347insG, 370C>T, 371G>A, 373G>A, 373_374insCG, 377T>A, 380delC, 386_387AC>TT, 387C>G, 389T>G, 406G>A, 413G>A, 442G>A, 443C>G, 457G>A, 470G>C, 1_150del150, 1_457del457, 151-1_151GG>AA, 151_151G>A, 151_457del307, 151_457del321, 458_471del14, (199_204)delG, 150+15T>C, 150+1G>A, 150+2T>C, 150+8G>C, 151−1G>A, 151−1G>T, 151−2A>G, 151−2A>T, 151−42C>T, 457+1G>A, and 458−2A>G.

In some instances, mutations in CDKN2A comprise deletions and/or mutations throughout the coding region. In some instances, a mutation in CDKN2A is in exon 1, 2, 3, 4, 5, 6, 7, 8, or a combination thereof of CDKN2A.

In some instances, a mutation in CDKN2A is a mutation in a peptide sequence. In some instances, the mutation results in a missense substitution, a nonsense substitution (*), a coding silent substitution, deletion (del), an insertion (ins), or a frameshift (fs). Exemplary mutations include, but are not limited to, E2K, P3_P11del, A4S, A4A, D14E, W15*, W15*, L16fs*9, A20P, G23S, G23fs*3, R24P, V28G, L31P, L32 L37del, L32 L37del, E33*, G35E, G35V, A36fs*17, A36G, A36T, L37L, P38L, S43fs*76, Y44fs*1, P48L, P48L, P48P, P48L, I49M, Q50*, Q50*, Q50*, Q50*, V51fs, M53I, M53fs*66, M54K, M54R, G55R, G55D, S56fs*64, A57fs*63, A57F, A57V, A57_R58>V*, A57A, R58*, R58fs*59, R58*, R58fs*62, R58fs*59, R58fs*88, R58*, V59G, A60V, A60fs*, E61fs*59, E61*, E61fs*52, L62P, L63P, L64L, L65P, H66*, G67*, G67fs*53, G67S, A68T, A68fs*51, A68fs*78, A68A, E69*, P70L, N71I, C72Y, A73V, A73A, D74E, P75S, P75fs*71, A76fs*64, A76V, T79I, T79T, R80*, R80*, R80fs*63, R80Q, R80*, R80, P81S, P81H, P81L, P81L, P81L, V82fs*44, V82fs*38, V82M, V82E, V82A, H83D, H83Y, H83R, H83L, H83P, H83Q, H83N, H83Y, D84N, D84_F90del, A85V, A86_R87>G, R87W, R87fs*59, R87Q, E87K, E88K, E88*, E88*, G89S, G89fs*57, F90L, F90F, V96_H98del, L97P, L97R, L97fs*21, R99W, R99R, A100P, A100V, G101W, G101W, A102T, A102V, L104R, R107C, R107H, A109V, W110*, W110*, W110*, W110*, G111S, R112G, R112P, P114H, P114L, P114L, P114P, P114L, D116Y, D116fs*4, R124C, R124H, D125N, D125fs*22, V126D, V126D, A127fs*19, Y129F, Y129*, L130R, L130R, G136S, R138K, A148T, A148G, D153N, and *157S. In some cases, the mutation(s) are at the corresponding residue positions as set forth in SEQ ID NO: 5.

In some instances, CDKN2A comprises one or more mutations in the protein region QVMMMGSARVAELLLLHGAEPNCADPATLTRPVHDAAREGFLDTLVVLHRAGARLDVRDAW GRLPVDLAEELGHRDVARYLRAAAGGTRGSNHARIDAAEGPS (SEQ ID NO: 6). In some cases, CDKN2A comprises a mutation at V51fs, M53I, R58*, E61*, G67*, E69*, or R80*, or a combination thereof, in which fs denotes frameshift and (*) denotes nonsense substitution.

TP53, also known as p53, cellular tumor antigen p53, phosphoprotein p53, tumor suppressor p53, antigen NY-CO-13, or transformation-related protein 53 (TRP53), encodes the tumor protein p53 (TP53). TP53 is a phosphoprotein made of 393 amino acids and comprises four domains. TP53 plays a role in cell cycle control and apoptosis. In some instances, a mutation in TP53 is associated with a non-melanoma skin cancer (e.g., BCC and/or SCC). In some instances, a mutation is a nucleotide sequence of TP53. For example, the mutation in the nucleotide sequence includes, but not limited to, 96+1G>A, 96+1G>T, 97-1G>A, 375+1G>A, 375+2T>C, 375_375+1GG>AT, 376-1G>A, 376-1G>T, 559+1G>A, 559+2T>G, 560-1G>A, 560-1G>T, 560-1_560GG>AA, 560-2A>C, 672+1G>A, 673-1G>A, 673-8T>A, 782+1G>C, 782_782+1GG>AA, 783-2A>T, 919+1G>A, 920-1G>T, 993+1G>A, 994-1G>A, 19G>C, 31G>A, 37C>T, 69G>A, 79C>T, 101C>T, 102C>G, 136T>C, 139_140delCC, 140delC, 142G>A, 151G>T, 158G>A, 159G>A, 159G>C, 162C>T, 166G>T, 173delC, 175_176GG>AA, 181G>A, 202G>A, 206C>T, 211C>T, 212C>T, 214C>G, 214C>T, 215C>G, 216_217insC, 217G>A, 229C>T, 238C>T, 239C>T, 242C>T, 242delC, 245C>T, 248C>A, 250G>A, 250_251insT, 251C>T, 251_252CC>TT, 253C>T, 254C>T, 257_279del23, 265C>T, 265_266CC>TT, 266C>T, 269C>T, 272G>A, 273G>A, 275C>T, 281C>T, 284C>T, 287C>T, 289G>A, 292C>T, 293C>T, 296C>T, 298C>T, 305C>T, 309C>G, 310C>T, 312delG, 313delG, 321C>A, 321C>G, 322G>A, 325_330delTTCCGT, 326T>C, 327_328CC>TT, 328C>T, 328delC, 332T>A, 349delG, 358A>G, 365_367delTGA, 375G>A, 375G>T, 380C>T, 380_381CC>TT, 382C>T, 386C>T, 388C>T, 388delC, 394A>C, 394A>G, 395A>T, 396G>T, 398T>A, 400T>C, 403T>C, 404G>A, 404G>C, 405C>A, 405_406CC>TT, 406C>T, 409C>A, 412delG, 413C>T, 413_414CC>TT, 415A>T, 416delA, 417G>C, 418_419insN, 419C>T, 424C>T, 424_425CC>TT, 425C>T, 428T>A, 428T>G, 430delC, 432G>A, 434_435TG>GT, 437G>A, 438G>A, 442_465del24, 446C>T, 447C>T, 449C>T, 451C>T, 452C>A, 452_453CC>TT, 453C>T, 453_454insN, 454C>T, 454_455CC>TT, 455C>G, 455C>T, 457_461delCCCGG, 456_457insC, 459_460insN, 463_464delAC, 465C>T, 466C>T, 466delC, 467G>C, 468_469delCG, 468_487del20, 469G>A, 469G>T, 471_472CC>TT, 472C>T, 474C>T, 475G>A, 476C>T, 476_477CC>TT, 477C>T, 480G>A, 480G>C, 481G>A, 482C>T, 483C>T, 487T>C, 487T>G, 493C>T, 496T>G, 502C>T, 502_503insN, 502_511del10, 507G>A, 508A>G, 509C>T, 511G>C, 513delG, 517G>A, 517G>C, 518T>G, 521G>A, 522G>C, 524G>A, 527G>A, 527G>T, 528C>G, 529C>T, 529_530CC>TT, 530C>T, 530_531CC>AT, 530_531CC>TT, 528delC, 531_532CC>TT, 532C>A, 532C>T, 532_533insN, 534_535CC>AT, 534_535CC>TT, 535C>A, 535C>T, 536A>T, 541C>T, 542G>A, 546C>T, 548C>A, 548C>T, 550G>A, 556G>A, 559G>A, 565G>A, 565_591del27, 566C>T, 567C>T, 568C>T, 568_569CC>TT, 569C>T, 571C>T, 572C>T, 573T>A, 574C>T, 580C>T, 581T>G, 582_586delTATCC, 583A>T, 585 586CC>TT, 586C>T, 587G>T, 590T>A, 592G>A, 592G>T, 599delA, 600_601insN, 601T>G, 603_604GC>TT, 603_604insAAATITG, 605G>C, 605G>T, 605_606GT>CG, 606delT, 613T>G, 614A>G, 617T>A, 620_627delATGACAGA, 622G>T, 626G>A, 626_627delGA, 632C>T, 637C>T, 638G>A, 638G>C, 640_647delCATAGTGT, 645T>G, 647T>G, 652G>A, 653T>A, 653T>G, 656C>T, 656_657CC>TT, 658T>A, 659A>G, 660_661insN, 662_672+40del51, 664_665CC>TT, 665C>T, 666G>C, 667delC, 670G>T, 674T>C, 677G>A, 677G>C, 680C>T, 682G>A, 683A>C, 685_686delTG, 688A>G, 689C>T, 690C>A, 691A>T, 697delC, 700T>A, 700T>C, 701A>C, 702C>A, 703A>G, 704A>G, 704A>T, 706T>A, 706T>C, 712T>A, 713G>A, 713G>T, 714T>G, 721T>C, 722C>T, 722_723CC>TT, 723C>T, 724T>A, 724T>G, 725G>A, 726C>T, 727A>T, 728T>G, 730G>A, 733G>A, 733G>T, 733_734GG>AA, 734G>A, 734G>T, 737T>C, 737T>G, 738G>A, 739A>T, 740A>C, 741_742CC>TT, 742C>T, 742_744CGG>TGC, 743G>A, 743G>C, 743G>T, 743_744GG>AA, 744G>A, 745A>T, 745_768del24, 746G>C, 746G>T, 747G>C, 747G>T, 748C>G, 748C>T, 748_749CC>TT, 749C>T, 749_750CC>TG, 750C>T, 752T>A, 755T>C, 756C>T, 757A>G, 758C>T, 759C>T, 762delC, 770T>C, 771_772GG>AA, 772G>A, 775delG, 776A>T, 781A>T, 791delT, 794T>C, 795G>A, 795_796GG>AA, 796G>A, 796G>T, 796_797GG>AA, 797G>A, 799C>T, 800G>C, 806G>A, 808T>A, 811G>A, 812_815delAGGT, 814G>A, 815T>A, 815T>G, 817C>G, 817C>T, 817_825delCGTGTITG . . . , 818G>A, 818G>T, 820G>A, 821T>C, 824G>A, 824G>C, 825T>G, 826G>C, 827C>A, 827C>T, 827_829CCT>TC, 829T>G, 830G>A, 830G>T, 832C>A, 832C>T, 832_833CC>TT, 833C>G, 833C>T, 834_835insN, 835G>A, 836G>A, 836_837GG>AA, 837G>A, 838A>T, 839G>A, 839G>C, 841G>A, 843C>A, 843C>G, 843C>T, 843_844CC>AT, 843_844CC>TT, 844C>G, 844C>T, 845G>A, 845G>T, 847C>T, 852A>T, 853G>A, 853G>C, 854A>T, 855G>A, 855_856GG>AA, 856G>A, 856G>C, 856G>T, 857A>T, 859G>A, 863delA, 865C>T, 867C>T, 868C>T, 868delC, 869G>A, 870C>G, 880G>A, 880G>T, 882G>A, 884C>T, 888_889CC>TT, 890delA, 892G>T, 898C>T, 899C>G, 898delC, 901C>T, 902_903insC, 904delG, 908G>C, 919G>T, 947C>T, 948_949CC>TT, 949C>T, 955A>G, 960G>A, 965C>T, 968T>C, 972T>A, 976G>T, 981T>A, 986_987CC>TT, 987C>A, 989T>G, 991C>T, 992_993insN, 1006G>T, 1009C>T, 1014C>T, 1023_1024CC>TT, 1024C>T, 1045G>T, 1050delC, 1051A>G, 1072G>A, 1082G>A, 1083delG, 1084delA, 1133C>T, 1143A>T, 559+11G>T, 559+37T>G, 74+12C>T, 783-57A>G, and 919+40delG.

In some instances, mutations in TP53 comprise deletions and mutations throughout the coding region. In some instances, a mutation in TP53 is in exon 1, 2, 3, 4, 5, 6, 7, 8, 9, 10, 11, or a combination thereof of TP53. In some instances, a mutation in TP53 is in exon 5, 7, 8, or a combination thereof of TP53. In some instances, a mutation in TP53 is in exon 5. In some instances, a mutation in TP53 is in exon 7. In some instances, a mutation in TP53 is in exon 8.

In some instances, a mutation in TP53 is a mutation in a peptide sequence. In some instances, the mutation results in a missense substitution, a nonsense substitution (*), a coding silent substitution, deletion (del), an insertion (ins), or a frameshift (fs). Exemplary mutations include, but are not limited to, D7H, E11K, P13S, W23*, P27S, V31I, P34L, P34P, S46P, S46F, P47fs*4, P47fs*76, D48N, E51*, W53*, W53*, W53C, F54F, E56*, P58fs*65, G59N, D61N, E68K, A69V, P71S, P71L, P72A, P72S, P72R, V73fs*76, V73M, P77S, P77del, P80S, P80L, T81I, T81fs*42, P82L, A83E, A84T, A84fs*65, A84V, A84V, P85S, P85L, A86fs*55, P87S, P89S, P89F, P89L, S90F, W91*, W91*, P92L, S94L, S95F, S96F, V97I, P98S, P98L, P98L, S99F, Q100*, T102I, Y103*, Q104*, Q104*, G105fs*18, G105fs*18, Y107*, Y107*, G108S, F109_R110delFR, F109S, R110C, R110C, R110fs*13, L111Q, T118fs*5, K120E, V122_T123>A, T125T, T125T, Y126*, S127F, S127F, S127F, P128S, A129V, L130F, L130fs*40, N131K, K132Q, K132E, K132M, K132N, M133K, F134L, C135R, C135Y, C135S, C135*, Q136*, Q136*, L137M, A138fs*32, A138V, A138V, K139*, K139fs*31, K139N, T140fs*9, T140I, P142S, P142F, P142L, V143E, V143G, Q144fs*26, Q144Q, L145R, W146*, W146*, V147A, D148_T155delDSTPP . . . , S149F, S149S, T150I, P151S, P151H, P151L, P151P, P152fs*29, P152S, P152L, P152R, P152L, P152L, P152S, P153fs*26, G154fs*27, G154fs*27, T155fs*25, T155T, R156C, R156fs*14, R156P, V157fs*23, V157fs*17, V157I, V157F, R158C, R158C, R158R, A159T, A159V, A159V, A159A, M160I, M160I, A161T, A161V, A161A, Y163H, Y163D, Q165*, S166A, H168Y, H168fs*13, H168fs*3, M1691, T170A, T170M, E171Q, V172fs*2, V173M, V173L, V173G, V173L, R174K, R174S, R175 (e.g., R175H), C176Y, C176F, C176W, P177S, P177F, P177L, P177H, P177L, P177S, H178fs*69, H178Y, H178N, H178Y, H178fs*3, H178_H179>QY, H179Y, H179N, H179Y, H179L, H179Y, R181C, R181H, C182C, S183*, S183L, D184N, D186N, G187S, G187D, A189T, A189_V197delAPPQHL, A189V, A189A, P190S, P190F, P190L, P191S, P191L, P191P, Q192*, L194F, L194R, I195fs*12, I195F, R196*, R196*, R196L, R196*, V197E, E198K, E198*, N200fs*47, L201fs*8, L201V, L201_R202>FC, R202fs*9, R202P, R202L, R202P, V203fs*44, Y205D, Y205C, L206*, D207fs*6, D208Y, R209K, R209fs*6, T211I, R213*, R213Q, R213P, H214fs*5, S215R, V216G, V216G, V216M, V218M, V218E, V218G, V218G, P219L, P219L, P219L, Y220N, Y220C, E221fs*4, E221fs*4, P222L, P222L, P222P, P223fs*24, E224*, V225A, G226D, G226A, S227F, D228N, D228A, C229fs*10, T230A, T2301, T230T, T231S, H233fs*14, Y234N, Y234H, Y234S, Y234*, Y234S, N235D, N235S, N235I, Y236N, Y236H, C238S, C238Y, C238F, C238W, S240, S241P, S241F, S241F, S241S, C242S, C242G, C242Y, C242C, M243L, M243R, G244S, G245 (e.g., G245S, G245C, G245N, G245D, G245V, G245D, G245R), M246T, M246R, M246I, N247Y, N247T, R248 (e.g., R248W, R248W, R248C, R248Q, R248P, R248L, R248Q, R248R, R248Q, R248W, R248Y), R249 (e.g., R249W, R249_T256delRPILTI . . . , R249T, R249M, R249S, R249S), P250A, P250S, P250F, P250L, P250L, P250P, I251N, L252P, L252L, T253A, T253I, T253T, I255fs*90, L257P, E258K, E258K, D259fs*86, D259V, D259Y, S261C, L264fs*81, L265P, L265L, G266R, G266R, G266*, G266K, G266E, R267W, R267P, S269N, F270I, E271K, E271fs*73, V272M, V272E, V272G, R273 (e.g., R273G, R273C, R273_C275delRVC, R273H, R273L, R273C, R273H), V274I, V274A, C275Y, C275S, C275W, A276P, A276D, A276V, A276fs*69, C277G, C277Y, C277F, P278T, P278S, P278F, P278R, P278L, P278F, P278S, G279fs*27, G279R, G279E, G279E, G279G, G279W, R280*, R280K, R280T, D281N, D281E, D281E, D281D, D281_R282>EW, R282 (e.g., R282W, R282G, R282W, R282Q, R282L), R283C, T284 (e.g., T284T), E285K, E285Q, E285V, E285E, E286K, E286K, E286Q, E286*, E286V, E287K, N288fs*57, L289F, L289L, R290C, R290fs*55, R290H, R290R, E294K, E294*, E294E, P295L, H297Y, H297fs*48, E298*, P300S, P300R, P301fs*44, P301S, G302fs*4, S303fs*42, S303T, A307S, P316L, Q317*, Q317*, Q317*, K319E, K320K, P322L, L323P, D324E, E326*, Y327*, T329I, T329T, L330R, Q331*, Q331fs*6, E336*, R337C, F338F, R342*, R342*, E349*, K351fs*19, K351E, E358K, G361E, S362fs*8, S362fs*8, T377P, S378F, and K381N. In some instances, a mutation is R175, S240, G245, R248, R249, R273, R282, T284, or combinations thereof. In some cases, the mutation(s) are at the corresponding residue positions as set forth in SEQ ID NO: 1.

PTCH1, also known as Patched 1 or Protein Patched Homolog 1, is a gene that encodes PTCH1, a member of the patched family of proteins. PTCH1 is involved in hedgehog signaling pathway. In some instances, mutations in PTCH1 are involved in a non-melanoma skin cancer (e.g., BCC and/or SCC). In some instances, a mutation is a nucleotide sequence of PTCH1. For example, the mutation in the nucleotide sequence includes, but not limited to, 394+1delG, 584_584+1GG>AA, 747-1G>A, 1067+1G>A, 1068-2A>T, 1068-2_1068-1AG>CT, 1216-4_1227del16, 1216-6C>T, 1347+1G>A, 1504-8T>C, 1603-1G>A, 1729-1G>T, 1847+3A>T, 1848-1G>A, 2251-1G>A, 2561-1G>A, 3168+5G>T, 3306+1G>A, 3449+1G>A, 3549+5G>A, 204G>A, 250C>A, 250C>T, 262_274del13, 271G>A, 272G>A, 277_278insA, 286A>T, 290_291insT, 292_310del19, 304T>A, 343_344GG>AA, 378delG, 387G>A, 394G>A, 404G>A, 426T>A, 430_431ins11, 441_442TG>AT, 445G>T, 451G>A, 463C>T, 475A>T, 478C>T, 493G>T, 523C>T, 528_529AC>CT, 549_550CC>TT, 550C>T, 584G>A, 584G>T, 631A>G, 652C>T, 654G>A, 666T>A, 681G>A, 707G>A, 708G>A, 708_709GG>AA, 709G>A, 712_713insA, 713G>A, 717G>C, 724C>T, 751_760del10, 754C>T, 757C>A, 757C>T, 758_776del19, 767G>A, 768G>A, 804_807delAAAG, 809_818del10, 813_819delAAACTAT, 833G>A, 834G>A, 838G>A, 851_872del22, 857_861AGGTT>G, 862G>A, 863G>A, 865delC, 864_871delTCATGGTT, 879_880CC>TT, 992C>T, 994A>T, 1031G>A, 1047delC, 1055G>A, 1062_1063insC, 1062_1063insT, 1082A>C, 1085C>T, 1092_1093CC>TT, 1093C>T, 1106_1107CC>TT, 1108_1111delAAGC, 1138G>T, 1160G>A, 1161G>A, 1167_1168GG>AT, 1196G>A, 1229G>A, 1249C>T, 1249_1250ins28, 1285delG, 1292T>A, 1316T>C, 1324delG, 1356T>G, 1361_1389del29, 1393_1394insC, 1396C>T, 1433C>T, 1434_1437delACTG, 1439C>G, 1450G>A, 1481C>T, 1481_1485delCCTTT, 1510C>T, 1511C>A, 1511C>T, 1557C>T, 1585A>T, 1594C>T, 1595C>T, 1615G>T, 1634G>A, 1667delT, 1673_1695del23, 1688C>T, 1703C>T, 1703_1704CC>TT, 1703_1711delCCGCT . . . , 1717T>A, 1719delC, 1721_1722CC>TT, 1722C>T, 1725C>T, 1726C>T, 1777_1778CC>TT, 1778C>T, 1796_1799delATTT, 1800A>T, 1804C>T, 1847G>A, 1847G>C, 1854C>A, 1863_1864delAG, 1887delC, 1893_1894insC, 1922C>T, 1930C>T, 1959_1969del11, 1977G>A, 1980C>T, 1986_1987CC>TT, 1992C>T, 1993C>G, 1993C>T, 2004C>T, 2008C>T, 2011C>T, 2020delG, 2033C>T, 2038G>T, 2042C>T, 2048C>T, 2050G>T, 2062C>T, 2066C>T, 2072C>T, 2105C>T, 2107G>T, 2120C>T, 2126G>A, 2128G>A, 2128delG, 2134_2144del11, 2146delT, 2147_2148CC>TT, 2178_2179insC, 2207C>T, 2209G>A, 2209G>T, 2265C>T, 2287G>A, 2287delG, 2307_2308CC>TT, 2308C>T, 2321G>A, 2334G>A, 2345C>T, 2364T>A, 2372T>C, 2380C>T, 2385_2399del15, 2397_2418del22, 2400C>T, 2421C>T, 2421_2422CC>TT, 2438C>A, 2439delG, 2446C>T, 2477delT, 2485G>A, 2492_2493insAGTA, 2557C>T, 2566_2568CAG>T, 2588G>A, 2589G>A, 2666A>G, 2693A>G, 2708_2709insAT, 2709_2710insAA, 2713C>T, 2716_2729del14, 2747_2748CC>AT, 2758_2771del14, 2765_2766ins14, 2777G>A, 2777_2778GG>AA, 2778G>A, 2778_2779GG>AA, 2791_2793CCC>T, 2793_2794ins22, 2794_2795insC, 2810C>T, 2812C>T, 2843G>A, 2847C>T, 2865C>A, 2866_2867delAT, 2873delA, 2885G>C, 2891_2892ins17, 2910delG, 2965G>T, 2974G>T, 2985G>T, 3027C>T, 3046C>T, 3054G>A, 3054_3055GG>AA, 3072C>T, 3120C>T, 3138C>T, 3148C>T, 3152_3153GG>AA, 3153G>A, 3196G>T, 3209T>G, 3236G>T, 3240C>T, 3249delG, 3261C>T, 3320_3321CC>TT, 3340A>T, 3356T>A, 3374_3375CC>TT, 3378_3379CC>TT, 3389C>T, 3401T>A, 3422C>T, 3425G>A, 3435C>T, 3487G>A, 3499G>A, 3499G>T, 3509_3538>GGA, 3514C>T, 3583A>T, 3584C>T, 3586C>T, 3590C>T, 3591C>T, 3592C>T, 3603C>T, 3605C>T, 3634G>A, 3641C>T, 3662C>T, 3708_3709GG>AA, 3715C>T, 3724G>A, 3739G>A, 3748C>T, 3815_3816CC>TT, 3833C>T, 3844C>T, 3856_3867del12, 3857C>T, 3859C>T, 3883C>T, 3906C>T, 3917C>T, 3918C>T, 3944T>C, 3970G>A, 4058C>T, 4140C>T, 4179C>T, 4187G>A, 4204C>T, 4205C>T, 4235C>T, 4249C>T, 4324C>T, 4328G>T, 1405_1406ins, 1728_1728+1delGG, 3169-1_3169GG>AA, 1503+3A>T, 1729-2A>T, 2250+25T>C, 3169-2A>G, 3450-1G>A, 3450-2A>T, 3550-27C>T, 394+1G>A, 584+5G>A, 654+1G>A, 654+2T>A, and 945+5G>C.

In some instances, mutations in PTCH1 comprise deletions and mutations throughout the coding region. In some instances, a mutation in PTCH1 is in exon 1, 2, 3, 4, 5, 6, 7, 8, 9, 10, 11, 12, 13, 14, 15, 16, 17, 18, 19, 20, 21, 22, 23, 24, or a combination thereof of PTCH1. In some instances, a mutation in PTCH1 is in exon 2, 3, 5, 6, 8, 10, 12, 14, 15, 17, 18, 22, 23, or a combination thereof of PTCH1. In some instances, a mutation in PTCH1 is in exon 14, 15, 17, or a combination thereof of PTCH1. In some instances, a mutation in PTCH1 is in exon 2, 3, 5, 6, 8, 10, 12, 18, 22, 23, or a combination thereof of PTCH1. In some instances, a mutation in PTCH1 is in exon 14, 15, 17, or a combination thereof and further in combination with one or more from exon 2, 3, 5, 6, 8, 10, 12, 18, 22, or 23. In some instances, a mutation in PTCH1 is in exon 2 of PTCH1. In some instances, a mutation in PTCH1 is in exon 3 of PTCH1. In some instances, a mutation in PTCH1 is in exon 5 of PTCH1. In some instances, a mutation in PTCH1 is in exon 6 of PTCH1. In some instances, a mutation in PTCH1 is in exon 8 of PTCH1. In some instances, a mutation in PTCH1 is in exon 10 of PTCH1. In some instances, a mutation in PTCH1 is in exon 12 of PTCH1. In some instances, a mutation in PTCH1 is in exon 14 of PTCH1. In some instances, a mutation in PTCH1 is in exon 15 of PTCH1. In some instances, a mutation in PTCH1 is in exon 17 of PTCH1. In some instances, a mutation in PTCH1 is in exon 18 of PTCH1. In some instances, a mutation in PTCH1 is in exon 22 of PTCH1. In some instances, a mutation in PTCH1 is in exon 23 of PTCH1.

In some instances, a mutation in PTCH1 is a mutation in a peptide sequence. In some instances, the mutation results in a missense substitution, a nonsense substitution (*), a coding silent substitution, deletion (del), an insertion (ins), or a frameshift (fs). Exemplary mutations include, but are not limited to, G68G, Q84K, Q84*, F88fs*25, G91S, G91D, Y93fs*1, K96*, C98fs*42, C98fs*13, L102M, G115K, E127fs*10, W129*, V132I, R135Q, Y142*, R144fs*19, G148*, E149*, A151T, P155S, I159L, Q160*, E165*, L175F, Q177*, Q184*, Q184*, R195K, R195M, T211A, Q218*, Q218Q, Y222*, L227L, W236*, W236*, W236_E237>*, E237K, G238fs*14, G238E, A239A, Q242*, K251fs*15, P252S, P253T, P253S, L254fs*9, W256*, W256*, K270fs*1, K270fs*10, N272fs*9, W278*, W278*, E280K, K284fs*33, E286fs*37, G288S, G288D, H289fs*35, Y291fs*25, R294C, S331F, R332*, G344D, N349fs*18, G352E, V355fs*82, V355fs*82, Q361P, T3621, Q365*, Q365*, P369L, K370fs*61, E380*, H384fs*, W387*, W387*, D390Y, W399*, S410N, Q417*, Q417fs*29, D429fs*3, L431Q, V439A, V442fs*14, Y452*, C454fs*1, Q466fs*31, Q466*, V469M, A478V, L479fs*11, S480*, A483G, G484R, S494F, S494fs*1, P504S, P504Q, P504L, A519A, K529*, P532S, P532L, E539*, G545E, V556fs*9, F559fs*60, A563V, P568L, P568L, P568_L570delPAL, F573I, S574fs*6, S574F, S574S, L575L, Q576*, P593F, P593L, P593L, L600fs*22, L600F, R602*, S616N, S616T, C618*, R621fs*5, Q628*, Y630fs*63, D632fs*22, P641L, P644S, E653fs*24, Q659Q, S660S, Q663*, L664L, R665G, R665C, E667*, Y668Y, P670S, H671Y, V674fs*19, T678I, E680*, P681L, S683F, E684*, Q688*, P689L, T691I, P702L, E703*, S707F, R709K, D710N, D710fs*36, S713fs*21, S716fs*30, S716F, C727fs*11, A736V, E737K, E737*, A741V, F755F, V763I, V763fs*9, R770*, R770*, G774E, T778T, P782L, Y788*, I791T, Q794*, K796_F800del, F800fs*23, F800F, T807T, Q808*, P813Q, N814fs*16, Q816*, F826fs*4, V829M, Y831*, Q853*, Q853*, Q856fs*1, W863*, W863*, Q889R, D898G, K904fs*21, Q905fs*20, Q905*, L907fs*4, P916H, Y920fs*34, Y922fs*1, W926*, W926*, W926*, W926_V927>*, W926*, P931fs*27, V932fs*34, V932fs*27, S937F, Q938*, W948*, V949V, Y955*, M956fs*2, T959fs*3, R962T, A965fs*36, E970fs*25, L981F, E989*, E992*, R995S, Y1009Y, Y1013*, L1016F, W1018*, W1018_E1019>*, L1024L, F1040F, F1046F, P1050S, W1051*, W1051*, E1066*, M1070R, S1079I, A1080A, V1084fs*3, 110871, A1107V, R1114W, L1119Q, P1125L, (=), A1130V, L1134Q, A1141V, G1142E, F1145F, G1163S, G1167R, G1167W, L1170_P1180>WT, P1172S, T1195S, T11951, P1196S, S1197F, S1197S, P1198S, P1201P, P1202L, G1212S, T1214M, S1221F, E1237K, R1239W, E1242K, E1242K, A1247T, P1250S, P1272L, P1278L, P1282S, P1286_D1289del, P1286L, H1287Y, P1295S, P1302P, P1306L, P1306P, L1315P, P1315L, E1324K, A1353V, A1380A, P1387S, P1393P, G1396E, P1402S, P1402L, P1412L, H1417Y, R1442W, and G1443V. In some instances, a mutation is S616N, S616T, C618*, R621fs*5, Y630fs*63, D632fs*22, P641L, P644S, E653fs*24, Q659Q, Q663*, L664L, R665G, Y668Y, P670S, H671Y, T678I, E680*, P681L, S683F, E684*, Q688*, P689L, T691I, P702L, S707F, S716fs*30, C727fs*11, A736V, or combinations thereof. In some cases, the mutation(s) are at the corresponding residue positions as set forth in SEQ ID NO: 3 or 4.

Expression level or mutational change once detected, in certain embodiments, provides information regarding a disease in an individual. In some instances, expression level of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, SPP1, or combinations thereof provides information regarding the disease in the individual. In some instances, mutational change of TERT, CDKN2A, TP53, PTCH1, or combinations thereof provides information regarding the disease of the individual. In some instances, both expression level and mutational change provide information regarding the disease in the individual. Information regarding the disease includes, but is not limited to, identification of a disease state, likelihood of treatment success for a given disease state, identification of progression of a disease state, and identification of a disease stage. In some instances, at least one of expression level and mutational change are compared to a control sample for identification of the disease state, determining likelihood of treatment success for the given disease state, identification of progression of the disease state, or identification of the disease stage. In some instances, the control sample is any sample that is used for making any one of these determinations. In some instances, the control sample is from a healthy individual. In some instances, the control is a sample from an individual with a known disease or disorder. In some instances, the control is from a database or reference. In some instances, the control is a normal sample from the same individual. In some instances, the normal sample is a sample that does not comprise cancer, disease, or disorder, or a sample that would test negative for cancer, disease, or disorder. In some instances, the normal sample is assayed at the same time or at a different time.

In some instances, an expression level of one or more genes of interest from a biological sample varies as compared to a control sample. In some instances, the control sample is a non-cancer sample. In some instances, the expression level is of at least two genes selected from a group consisting of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, and SPP1. In some instances, the expression level is at least or about 5%, 6%, 7%, 8%, 9%, 10%, 11%, 12%, 13%, 14%, 15%, 16%, 17%, 18%, 19%, 20%, 22%, 24%, 28%, 30%, 35%, 40%, 45%, 50%, 55%, 60%, 65%, 70%, 75%, 80%, 85%, 90%, 95% or more than 95% increased as compared to control. In some instances, the expression level is increased in a range of about 1% to about 100%, about 10% to about 90%, about 20% to about 80%, about 30% to about 70%, or about 40% to about 60%.

In some instances, a mutational change in one or more genes of interest from a biological sample comprises at least one mutation as compared to a control sample. In some instances, the mutational change is in TERT, CDKN2A, TP53, PTCH1, or a combination thereof. In some instances, TERT, CDKN2A, TP53, PTCH1, or a combination thereof from the biological sample comprises at least or about 1, 2, 3, 4, 5, 6, 7, 8, 9, 10, or more than 10 mutations. In some instances, TERT, CDKN2A, TP53, PTCH1, or a combination thereof from the biological sample comprises at least or about at least 1.5×, 2×, 3×, 4×, 5×, 6×, 7×, 8×, 9×, 10×, 11×, or 12× more mutations compared to a normal biological sample.

In some instances, at least one of expression level and mutational change of a gene of interest provide information regarding a skin cancer. In some instances, the skin cancer is melanoma, basal cell carcinoma (BCC), or squamous cell carcinoma (SCC). In some instances, the skin cancer is BCC. In some instances, the skin cancer is SCC. For example, the at least one of expression level and mutational change of a gene of interest provide information regarding a stage of skin cancer. In some instances, the at least one of expression level and mutational change of a gene of interest is associated with a stage of skin cancer. In some instances, one or more mutations in a gene of interest indicate a risk factor for skin cancer or the stage of skin cancer. In some instances, the gene of interest is MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, and SPP1. In some instances, the gene of interest is at least one of TERT, CDKN2A, TP53, and PTCH1.

Methods and compositions provided herein comprising detecting at least one of expression level and mutational change result in improved sensitivity and specificity for diagnosis or prognosis of disease. In some instances, detecting at least one of expression level and mutational change result in improved sensitivity and specificity for diagnosis or prognosis of skin cancer. In some instances, sensitivity is improved by at least or about 55%, 60%, 65%, 70%, 75%, 80%, 85%, 90%, 95% or more than 95% as compared to other diagnosis or prognosis methods. In some instances, specificity is improved by at least or about 55%, 60%, 65%, 70%, 75%, 80%, 85%, 90%, 95% or more than 95% as compared to other diagnosis or prognosis methods. The other diagnosis or prognosis methods include, but are not limited to, morphology histopathology, pattern histopathology, and RNA only based gene expression assays.

### Computer Implemented Methods and Systems for Carcinoma Assay

Described herein, in some embodiments, are computer-implemented methods for differentiating one or more cancer samples from one or more non-cancer samples, comprising: (a) hybridizing a set of probes that recognizes MMP1, S100A7, CMPK2, IRF7, IGFL1, CXCL1, UPP1, DEFB4A, FOS, OAS3, SCD5, RTP4, VEGFA, COL5A2, 1L24, AADACL2, PTCH1, CD68, PRKACA, or SPP1 to generate gene expression data; (b) obtaining, by a processor, the gene expression data; and (c) analyzing, by the processor, the gene expression data to differentiate the one or more cancer samples from the one or more non-cancer samples, wherein the analysis comprises: (i) generating a plurality of pair-wise interactions between at least two genes of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, and SPP1 in a set of samples; (ii) analyzing the plurality of pair-wise interactions using a machine learning method to determine an area under a curve (AUC) value for each of the plurality of pair-wise interactions; and (iii) differentiating the one or more cancer samples from the one or more non-cancer samples when an AUC value is greater than above about 0.8. In some instances, the pair-wise interactions are set out in Table 1. In some instances, the pair-wise interactions are set out in Table 2. In some instances, the pair-wise interactions are set out in Table 3. In some instances, the pair-wise interactions comprises MMP1, S100A7; CMPK2, IRF7; IGFL1, CXCL1; and IGFL1, CMPK2. In some instances, the pair-wise interactions comprises MMP1, S100A7; CMPK2, IRF7; IGFL1, CXCL1; IGFL1, CMPK2; and UPP1, CMPK2. In some instances, the pair-wise interactions comprises SCD5, S100A7 and CMPK2, IRF7. In some instances, the pair-wise interactions comprises IGFL1, COL5A2; IL24, AADACL2; PTCH1, CD68; PRKACA, SPP1; AADACL2, MMP1; or a combination thereof. In some instances, the pair-wise interactions comprises IGFL1, COL5A2 or AADACL2, MMP1. In some instances, the pair-wise interactions comprises IL24, AADACL2; PTCH1, CD68; or PRKACA, SPP1. In some instances, the pair-wise interactions comprises IGFL1, COL5A2; IL24, AADACL2; PTCH1, CD68; PRKACA, SPP1; or AADACL2, MMP1. In some instances, the pair-wise interactions comprises IGFL1, COL5A2; IL24, AADACL2; PTCH1, CD68; PRKACA, SPP1; and AADACL2, MMP1. In some instances, the machine learning method utilizes an algorithm selected from: random forest (rf) model, boosting model, logit model, or lasso model. In some instances, the set of samples comprises basal cell carcinoma, squamous cell carcinoma, actinic keratosis (AK), seborrheic keratosis (SK), normal samples, or a combination thereof. In some instances, the set of samples comprises basal cell carcinoma, squamous cell carcinoma, actinic keratosis (AK), seborrheic keratosis (SK), and normal samples. In some instances, the set of samples comprises basal cell carcinoma, squamous cell carcinoma, and actinic keratosis (AK). In some instances, the set of samples comprises basal cell carcinoma and squamous cell carcinoma. In some instances, the one or more cancer samples are differentiated from the one or more non-cancer samples when an AUC value is greater than above about 0.6, 0.65, 0.7, 0.75, 0.8, 0.85, 0.9, or 0.95.

Described herein, in some embodiments, are computer-implemented systems comprising a digital processing device comprising: (a) a first computing device comprising a processor, a memory module, an operating system, and a computer program including instructions executable by the processor to create a data acquisition application for receiving gene expression from a sample, the data acquisition application comprising: a data receiving module for receiving gene expression data of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, SPP1, or a combination thereof; and (b) a second computing device comprising a processor, a memory module, an operating system, and a computer program including instructions executable by the processor to create a data analysis application for differentiating a cancer sample from a non-cancer sample, the data analysis application comprising a data analysis module to: (i) generate a plurality of pair-wise interactions between at least two genes of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, and SPP1 from the gene expression data; and (ii) analyze the plurality of pair-wise interactions with a control using a machine learning method to differentiate a cancer sample from a non-cancer sample, wherein the machine learning method comprises: (1) identifying a plurality of weights associated with the plurality of pair-wise interactions based on a top score; (2) determining an area under a curve (AUC) value for each of the plurality of pair-wise interactions; and (3) classifying the sample as a cancer or non-cancer sample based on the plurality of pair-wise interactions and the plurality of weights when the AUC value is greater than about 0.8. In some instances, the pair-wise interactions are set out in Table 1. In some instances, the pair-wise interactions are set out in Table 2. In some instances, the pair-wise interactions are set out in Table 3. In some instances, the pair-wise interactions comprises MMP1, S100A7; CMPK2, IRF7; IGFL1, CXCL1; and IGFL1, CMPK2. In some instances, the pair-wise interactions comprises MMP1, S100A7; CMPK2, IRF7; IGFL1, CXCL1; IGFL1, CMPK2; and UPP1, CMPK2. In some instances, the pair-wise interactions comprises SCD5, S100A7 and CMPK2, IRF7. In some instances, the pair-wise interactions comprises IGFL1, COL5A2; IL24, AADACL2; PTCH1, CD68; PRKACA, SPP1; AADACL2, MMP1; or a combination thereof. In some instances, the pair-wise interactions comprises IGFL1, COL5A2 or AADACL2, MMP1. In some instances, the pair-wise interactions comprises IL24, AADACL2; PTCH1, CD68; or PRKACA, SPP1. In some instances, the pair-wise interactions comprises IGFL1, COL5A2; IL24, AADACL2; PTCH1, CD68; PRKACA, SPP1; or AADACL2, MMP1. In some instances, the pair-wise interactions comprises IGFL1, COL5A2; IL24, AADACL2; PTCH1, CD68; PRKACA, SPP1; and AADACL2, MMP1. In some instances, the machine learning method utilizes an algorithm selected from: random forest (rf) model, boosting model, logit model, or lasso model. In some instances, the machine learning method utilizes rf model, boosting model, or lasso model. In some instances, the control dataset comprises a first set of pair-wise interactions between at least two genes of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, and SPP1 from gene expression data obtained from at least one cancer sample; and a second set of pair-wise interactions between at least two genes of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, and SPP1 obtained from at least one non-cancer sample. In some instances, the at least one cancer sample comprises basal cell carcinoma, squamous cell carcinoma, or a combination thereof. In some instances, the at least one non-cancer sample comprises actinic keratosis (AK), seborrheic keratosis (SK), a normal sample, or a combination thereof. In some instances, the gene expression data comprises one or more cycle threshold (Ct) values. In some instances, the data receiving module further receives gene expression data from a set of samples. In some instances, the set of samples comprises: basal cell carcinoma, squamous cell carcinoma, actinic keratosis (AK), seborrheic keratosis (SK), a normal sample, or a combination thereof; or basal cell carcinoma, squamous cell carcinoma, and actinic keratosis (AK). In some instances, the one or more cancer samples are differentiated from the one or more non-cancer samples when an AUC value is greater than above about 0.6, 0.65, 0.7, 0.75, 0.8, 0.85, 0.9, or 0.95.

Described herein, in some embodiments, are non-transitory computer-readable medium with instructions stored thereon, that when executed by a processor, perform the steps comprising: (a) generating gene expression data of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, and SPP1 from a set of biological samples by a hybridizing method; (b) obtaining, by a processor, the gene expression data; (c) generating a plurality of pair-wise interactions between at least two genes of MMP1, S100A7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, and SPP1; (d) analyzing the plurality of pair-wise interactions using a machine learning method to determine an area under a curve (AUC) value for each of the plurality of pair-wise interactions; and (e) differentiating the one or more cancer samples from the one or more non-cancer samples when an AUC value is greater than above about 0.8. In some instances, the pair-wise interactions are set out in Table 1. In some instances, the pair-wise interactions are set out in Table 2. In some instances, the pair-wise interactions are set out in Table 3. In some instances, the pair-wise interactions comprises MMP1, S100A7; CMPK2, IRF7; IGFL1, CXCL1; and IGFL1, CMPK2. In some instances, the pair-wise interactions comprises MMP1, S100A7; CMPK2, IRF7; IGFL1, CXCL1; IGFL1, CMPK2; and UPP1, CMPK2. In some instances, the pair-wise interactions comprises SCD5, S100A7 and CMPK2, IRF7. In some instances, the pair-wise interactions comprises IGFL1, COL5A2; IL24, AADACL2; PTCH1, CD68; PRKACA, SPP1; AADACL2, MMP1; or a combination thereof. In some instances, the pair-wise interactions comprises IGFL1, COL5A2 or AADACL2, MMP1. In some instances, the pair-wise interactions comprises IL24, AADACL2; PTCH1, CD68; or PRKACA, SPP1. In some instances, the pair-wise interactions comprises IGFL1, COL5A2; IL24, AADACL2; PTCH1, CD68; PRKACA, SPP1; or AADACL2, MMP1. In some instances, the pair-wise interactions comprises IGFL1, COL5A2; IL24, AADACL2; PTCH1, CD68; PRKACA, SPP1; and AADACL2, MMP1. In some instances, the machine learning method utilizes an algorithm selected from: random forest (rf) model, boosting model, logit model, or lasso model. In some instances, the set of samples comprises basal cell carcinoma, squamous cell carcinoma, actinic keratosis (AK), seborrheic keratosis (SK), normal samples, or a combination thereof. In some instances, the set of samples comprises basal cell carcinoma, squamous cell carcinoma, actinic keratosis (AK), seborrheic keratosis (SK), and normal samples. In some instances, the set of samples comprises basal cell carcinoma, squamous cell carcinoma, and actinic keratosis (AK). In some instances, the one or more cancer samples are differentiated from the one or more non-cancer samples when an AUC value is greater than above about 0.6, 0.65, 0.7, 0.75, 0.8, 0.85, 0.9, or 0.95.

### Biological Samples and Methods of Use

Biological samples are obtained using a variety of methods. In some instances, obtaining a biological sample such as a skin sample comprises, but is not limited to, scraping of the skin, biopsy, suction, blowing and other techniques. In some instances, obtaining the biological sample is non-invasive. For example, the biological sample is obtained from a skin using a skin sample collector. In some cases, the biological sample is obtained by applying an adhesive patch to a skin sample in a manner sufficient to adhere a sample of the skin to the adhesive patch, and removing the adhesive patch from the skin in a manner sufficient to retain the adhered skin sample to the adhesive patch. In some instances, the patch comprises a rubber adhesive on a polyurethane film. In some instances, about one to about ten adhesive patches or one to ten applications of the patch are applied to and removed from the skin.

In some instances, an effective amount of skin sample is removed by the adhesive patch. In some instances, the effective amount comprises between about 50 microgram to about 500 microgram, between about 100 microgram to about 450 microgram, between about 100 microgram to about 350 microgram, between about 100 microgram to about 300 microgram, between about 120 microgram to about 250 microgram, or between about 150 microgram to about 200 microgram of nucleic acid material.

In some instances, the adhesive patch comprises various materials. In some embodiments, the adhesive patch comprises a matrix comprising a synthetic rubber compound. In some embodiments, the adhesive patch does not comprise a latex material, a silicone material, or a combination thereof.

In some embodiments, the adhesive patch comprises a first central collection area having a skin facing surface comprising the adhesive matrix and a second area extending from the periphery of the first collection area creating a tab. In some cases, the first central collection area and the second area are comprised of different materials. In some cases, the first central collection area is comprised of a polyurethane carrier film.

In some embodiments, the skin sample is obtained from a site on a body. In some instances, the skin sample is obtained from a chest, forehead, hand, mastoid, temple, abdomen, arm, or leg. In some cases, the skin sample is not obtained from an area located on the palms, soles of feet, or mucous membranes.

In some embodiments, the skin sample is obtained from a skin lesion. In some cases, the skin lesion is a pigmented skin lesion comprising a mole, dark colored skin spot, or melanin containing skin area. In some cases, the skin lesion is an area on the skin surface that is suspicious for melanoma, lupus, rubeola, acne, hemangioma, psoriasis, eczema, candidiasis, impetigo, shingles, leprosy, Crohn's disease, inflammatory dermatoses, bullous diseases, infections, basal cell carcinoma, actinic keratosis, merkel cell carcinoma, sebaceous carcinoma, squamous cell carcinoma, and dermatofibrosarcoma protuberans. In some instances, the skin lesion is suspicious for skin cancer. Exemplary skin cancer include, but are not limited to, melanoma, basal cell carcinoma (BCC), squamous cell carcinoma (SCC), angiosarcoma, cutaneous B-cell lymphoma, cutaneous T-cell lymphoma, dermatofibrosarcoma protuberans, Merkel cell carcinoma, and sebaceous gland carcinoma. In some instances, the skin lesion is suspicious for melanoma. In some instances, the skin lesion is suspicious for basal cell carcinoma. In some instances, the skin lesion is suspicious for squamous cell carcinoma.

In some cases, the skin lesion is from about 5 mm to about 20 mm in diameter.

Methods and compositions as described herein, in certain embodiments, result in obtaining various layers of skin. In some instances, the layers of skin include epidermis or dermis. The epidermis is further subdivided into stratum corneum, stratum lucidum, stratum granulosum, stratum spinosum, and stratum germinativum. In some instances, the skin sample is obtained from the epidermis layer, optionally from one or more of stratum corneum, stratum lucidum, stratum granulosum, stratum spinosum, and stratum germinativum. In some instances, the skin sample is obtained from the dermis layer. In some instances, cells are obtained from the skin using methods and compositions as described herein. Exemplary cells that are obtained include, but are not limited to, keratinocytes, melanocytes, basal cells, T-cells, Merkel cells, Langerhans cells, fibroblasts, macrophages, adipocytes, and dendritic cells.

Provided herein are methods and compositions for extraction of nucleic acids from a biological sample such as a sample collected using an adhesive patch. In some instances, nucleic acids are extracted using any technique that does not interfere with subsequent analysis. In some instances, this technique uses alcohol precipitation using ethanol, methanol or isopropyl alcohol. In some instances, this technique uses phenol, chloroform, or any combination thereof. In some instances, this technique uses cesium chloride. In some instances, this technique uses sodium, potassium or ammonium acetate or any other salt commonly used to precipitate the nucleic acids.

In some instances, the nucleic acid is a RNA molecule or a fragmented RNA molecule (RNA fragments). In some instances, the RNA is a microRNA (miRNA), a pre-miRNA, a pri-miRNA, a mRNA, a pre-mRNA, a viral RNA, a viroid RNA, a virusoid RNA, circular RNA (circRNA), a ribosomal RNA (rRNA), a transfer RNA (tRNA), a pre-tRNA, a long non-coding RNA (lncRNA), a small nuclear RNA (snRNA), a circulating RNA, a cell-free RNA, an exosomal RNA, a vector-expressed RNA, a RNA transcript, a synthetic RNA, or combinations thereof. In some instances, the RNA is mRNA. In some instances, the RNA is cell-free circulating RNA.

In some instances, the nucleic acid is DNA DNA includes, but not limited to, genomic DNA, viral DNA, mitochondrial DNA, plasmid DNA, amplified DNA, circular DNA, circulating DNA, cell-free DNA, or exosomal DNA. In some instances, the DNA is single-stranded DNA (ssDNA), double-stranded DNA, denaturing double-stranded DNA, synthetic DNA, and combinations thereof. In some instances, the DNA is genomic DNA. In some instances, the DNA is cell-free circulating DNA.

Following extraction of nucleic acids from a biological sample, the nucleic acids, in some instances, are further purified. In some instances, the nucleic acids are RNA. In some instances, the nucleic acids are DNA. In some instances, nucleic acids are purified using a column or resin based nucleic acid purification scheme. In some instances, this technique utilizes a support comprising a surface area for binding the nucleic acids. In some instances, the support is made of glass, silica, latex or a polymeric material. In some instances, the support comprises spherical beads.

Methods and compositions for isolating nucleic acids, in certain embodiments, comprise using spherical beads. In some instances, the beads comprise material for isolation of nucleic acids. Exemplary material for isolation of nucleic acids using beads include, but not limited to, glass, silica, latex, and a polymeric material. In some instances, the beads are magnetic. In some instances, the beads are silica coated. In some instances, the beads are silica-coated magnetic beads. In some instances, a diameter of the spherical bead is at least or about 0.5 um, 1 um, 1.5 um, 2 um, 2.5 um, 3 um, 3.5 um, 4 um, 4.5 um, 5 um, 5.5 um, 6 um, 6.5 um, 7 um, 7.5 um, 8 um, 8.5 um, 9 um, 9.5 um, 10 um, or more than 10 um.

In some cases, a yield of the nucleic acids products obtained using methods described herein is about 500 picogram or higher, about 1000 picogram or higher, about 2000 picogram or higher, about 3000 picogram or higher, about 4000 picogram or higher, about 5000 picogram or higher, about 6000 picogram or higher, about 7000 picogram or higher, about 8000 picogram or higher, about 9000 picogram or higher, about 10000 picogram or higher, about 20000 picogram or higher, about 30000 picogram or higher, about 40000 picogram or higher, about 50000 picogram or higher, about 60000 picogram or higher, about 70000 picogram or higher, about 80000 picogram or higher, about 90000 picogram or higher, or about 100000 picogram or higher.

In some cases, methods described herein provide less than less than 10%, less than 8%, less than 5%, less than 2%, less than 1%, or less than 0.5% product yield variations between samples.

In some cases, methods described herein provide a substantially homogenous population of a nucleic acid product.

In some cases, methods described herein provide less than 30%, less than 25%, less than 20%, less than 15%, less than 10%, less than 8%, less than 5%, less than 2%, less than 1%, or less than 0.5% contaminants.

In some instances, following extraction, nucleic acids are stored. In some instances, the nucleic acids are stored in water, Tris buffer, or Tris-EDTA buffer before subsequent analysis. In some instances, this storage is less than 8° C. In some instances, this storage is less than 4° C. In certain embodiments, this storage is less than 0° C. In some instances, this storage is less than −20° C. In certain embodiments, this storage is less than −70° C. In some instances, the nucleic acids are stored for about 1, 2, 3, 4, 5, 6, or 7 days. In some instances, the nucleic acids are stored for about 1, 2, 3, or 4 weeks. In some instances, the nucleic acids are stored for about 1, 2, 3, 4, 5, 6, 7, 8, 9, 10, 11, or 12 months.

In some instances, nucleic acids isolated using methods described herein are subjected to an amplification reaction following isolation and purification. Non-limiting amplification reactions include, but are not limited to, quantitative PCR (qPCR), self-sustained sequence replication, transcriptional amplification system, Q-Beta Replicase, rolling circle replication, or any other nucleic acid amplification known in the art. In some instances, the amplification reaction is PCR. In some instances, the amplification reaction is quantitative such as qPCR.

Provided herein are methods and compositions for detecting an expression level of one or more genes of interest from nucleic acids isolated from a biological sample. In some instances, the expression level is detected following an amplification reaction. In some instances, the nucleic acids are RNA. In some instances, the expression level is determined using PCR. In some instances, the expression level is determined using qPCR. In some instances, primers and probes for use in the qPCR are specific to MMP1, S100A 7, CMPK2, IRF7, IGFL1, CXCL1, UPP1, DEFB4A, FOS, OAS3, SCD5, RTP4, VEGFA, COL5A2, IL24, AADACL2, PTCH1, CD68, PRKACA, SPP1, or a combination thereof. In some instances, the expression level is determined using a microarray. In some instances, the expression level is determined by sequencing.

Provided herein are methods and compositions for detecting a mutational change of one or more genes of interest from nucleic acids isolated from a biological sample. In some instances, the mutational change is detected following an amplification reaction. In some instances, the nucleic acids are RNA. In some instances, the nucleic acids are DNA. In some instances, the mutational change is detected using allele specific PCR. In some instances, the mutational change is detected using sequencing. In some instances, the sequencing is performed using the Sanger sequencing method. In some instances, the sequencing involves the use of chain terminating dideoxynucleotides. In some instances, the sequencing involves gel-electrophoresis. In some instances, the sequencing is performed using a next generation sequencing method. In some instances, sequencing includes, but not limited to, single-molecule real-time (SMRT) sequencing, Polony sequencing, sequencing by synthesis, sequencing by ligation, reversible terminator sequencing, proton detection sequencing, ion semiconductor sequencing, nanopore sequencing, electronic sequencing, pyrosequencing, Maxam-Gilbert sequencing, chain termination sequencing, +S sequencing, and sequencing by synthesis.

### Components of the Skin Collection Kit

In some embodiments, the adhesive patch from the sample collection kit described herein comprises a first collection area comprising an adhesive matrix and a second area extending from the periphery of the first collection area. The adhesive matrix is located on a skin facing surface of the first collection area. The second area functions as a tab, suitable for applying and removing the adhesive patch. The tab is sufficient in size so that while applying the adhesive patch to a skin surface, the applicant does not come in contact with the matrix material of the first collection area. In some embodiments, the adhesive patch does not contain a second area tab. In some instances, the adhesive patch is handled with gloves to reduce contamination of the adhesive matrix prior to use.

In some embodiments, the first collection area is a polyurethane carrier film. In some embodiments, the adhesive matrix is comprised of a synthetic rubber compound. In some embodiments, the adhesive matrix is a styrene-isoprene-styrene (SIS) linear block copolymer compound. In some instances, the adhesive patch does not comprise latex, silicone, or both. In some instances, the adhesive patch is manufactured by applying an adhesive material as a liquid-solvent mixture to the first collection area and subsequently removing the solvent.

The matrix material is sufficiently sticky to adhere to a skin sample. The matrix material is not so sticky that is causes scarring or bleeding or is difficult to remove. In some embodiments, the matrix material is comprised of a transparent material. In some instances, the matrix material is biocompatible. In some instances, the matrix material does not leave residue on the surface of the skin after removal. In certain instances, the matrix material is not a skin irritant.

In some embodiments, the adhesive patch comprises a flexible material, enabling the patch to conform to the shape of the skin surface upon application. In some instances, at least the first collection area is flexible. In some instances, the tab is plastic. In an illustrative example, the adhesive patch does not contain latex, silicone, or both. In some embodiments, the adhesive patch is made of a transparent material, so that the skin sampling area of the subject is visible after application of the adhesive patch to the skin surface. The transparency ensures that the adhesive patch is applied on the desired area of skin comprising the skin area to be sampled. In some embodiments, the adhesive patch is between about 5 and about 100 mm in length. In some embodiments, the first collection area is between about 5 and about 40 mm in length. In some embodiments, the first collection area is between about 10 and about 20 mm in length. In some embodiments the length of the first collection area is configured to accommodate the area of the skin surface to be sampled, including, but not limited to, about 19 mm, about 20 mm, about 21 mm, about 22 mm, about 23 mm, about 24 mm, about 25 mm, about 30 mm, about 35 mm, about 40 mm, about 45 mm, about 50 mm, about 55 mm, about 60 mm, about 65 mm, about 70 mm, about 75 mm, about 80 mm, about 85 mm, about 90 mm, and about 100 mm. In some embodiments, the first collection area is elliptical.

In further embodiments, the adhesive patch of this invention is provided on a peelable release sheet in the adhesive skin sample collection kit. In some embodiments, the adhesive patch provided on the peelable release sheet is configured to be stable at temperatures between −80° C. and 30° C. for at least 6 months, at least 1 year, at least 2 years, at least 3 years, and at least 4 years. In some instances, the peelable release sheet is a panel of a tri-fold skin sample collector.

In some instances, nucleic acids are stable on adhesive patch or patches when stored for a period of time or at a particular temperature. In some instances, the period of time is at least or about 1 day, 2 days, 3 days, 4 days, 5 days, 6 days, 7 days, 2 weeks, 3 weeks, 4 weeks, or more than 4 weeks. In some instances, the period of time is about 7 days. In some instances, the period of time is about 10 days. In some instances, the temperature is at least or about −80° C., −70° C., −60° C., −50° C., −40° C., −20° C., −10° C., −4° C., 0° C., 5° C., 15° C., 18° C., 20° C., 25° C., 30° C., 35° C., 40° C., 45° C., 50° C., or more than 50° C. The nucleic acids on the adhesive patch or patches, in some embodiments, are stored for any period of time described herein and any particular temperature described herein. For example, the nucleic acids on the adhesive patch or patches are stored for at least or about 7 days at about 25° C., 7 days at about 30° C., 7 days at about 40° C., 7 days at about 50° C., 7 days at about 60° C., or 7 days at about 70° C. In some instances, the nucleic acids on the adhesive patch or patches are stored for at least or about 10 days at about −80° C.

The peelable release sheet, in certain embodiments, is configured to hold a plurality of adhesive patches, including, but not limited to, 12, 11, 10, 9, 8, 7, 6, 5, 4, 3, 2, 1, from about 2 to about 8, from about 2 to about 7, from about 2 to about 6, from about 2 to about 4, from about 3 to about 6, from about 3 to about 8, from about 4 to about 10, from about 4 to about 8, from about 4 to about 6, from about 4 to about 5, from about 6 to about 10, from about 6 to about 8, or from about 4 to about 8. In some instances, the peelable release sheet is configured to hold about 12 adhesive patches. In some instances, the peelable release sheet is configured to hold about 11 adhesive patches. In some instances, the peelable release sheet is configured to hold about 10 adhesive patches. In some instances, the peelable release sheet is configured to hold about 9 adhesive patches. In some instances, the peelable release sheet is configured to hold about 8 adhesive patches. In some instances, the peelable release sheet is configured to hold about 7 adhesive patches. In some instances, the peelable release sheet is configured to hold about 6 adhesive patches. In some instances, the peelable release sheet is configured to hold about 5 adhesive patches. In some instances, the peelable release sheet is configured to hold about 4 adhesive patches. In some instances, the peelable release sheet is configured to hold about 3 adhesive patches. In some instances, the peelable release sheet is configured to hold about 2 adhesive patches. In some instances, the peelable release sheet is configured to hold about 1 adhesive patch.

Provided herein, in certain embodiments, are methods and compositions for obtaining a sample using an adhesive patch, wherein the adhesive patch is applied to the skin and removed from the skin. After removing the used adhesive patch from the skin surface, the patch stripping method, in some instances, further comprise storing the used patch on a placement area sheet, where the patch remains until the skin sample is isolated or otherwise utilized. In some instances, the used patch is configured to be stored on the placement area sheet for at least 1 week at temperatures between −80° C. and 30° C. In some embodiments, the used patch is configured to be stored on the placement area sheet for at least 2 weeks, at least 3 weeks, at least 1 month, at least 2 months, at least 3 months, at least 4 months, at least 5 months, and at least 6 months at temperatures between −80° C. to 30° C.

In some instances, the placement area sheet comprises a removable liner, provided that prior to storing the used patch on the placement area sheet, the removable liner is removed. In some instances, the placement area sheet is configured to hold a plurality of adhesive patches, including, but not limited to, 12, 11, 10, 9, 8, 7, 6, 5, 4, 3, 2, 1, from about 2 to about 8, from about 2 to about 7, from about 2 to about 6, from about 2 to about 4, from about 3 to about 6, from about 3 to about 8, from about 4 to about 10, from about 4 to about 8, from about 4 to about 6, from about 4 to about 5, from about 6 to about 10, from about 6 to about 8, or from about 4 to about 8. In some instances, the placement area sheet is configured to hold about 12 adhesive patches. In some instances, the placement area sheet is configured to hold about 11 adhesive patches. In some instances, the placement area sheet is configured to hold about 10 adhesive patches. In some instances, the placement area sheet is configured to hold about 9 adhesive patches. In some instances, the placement area sheet is configured to hold about 8 adhesive patches. In some instances, the placement area sheet is configured to hold about 7 adhesive patches. In some instances, the placement area sheet is configured to hold about 6 adhesive patches. In some instances, the placement area sheet is configured to hold about 5 adhesive patches. In some instances, the placement area sheet is configured to hold about 4 adhesive patches. In some instances, the placement area sheet is configured to hold about 3 adhesive patches. In some instances, the placement area sheet is configured to hold about 2 adhesive patches. In some instances, the placement area sheet is configured to hold about 1 adhesive patch.

The used patch, in some instances, is stored so that the matrix containing, skin facing surface of the used patch is in contact with the placement area sheet. In some instances, the placement area sheet is a panel of the tri-fold skin sample collector. In some instances, the tri-fold skin sample collector further comprises a clear panel. In some instances, the tri-fold skin sample collector is labeled with a unique barcode that is assigned to a subject. In some instances, the tri-fold skin sample collector comprises an area for labeling subject information.

In an illustrative embodiment, the adhesive skin sample collection kit comprises the tri-fold skin sample collector comprising adhesive patches stored on a peelable release panel. In some instances, the tri-fold skin sample collector further comprises a placement area panel with a removable liner. In some instances, the patch stripping method involves removing an adhesive patch from the tri-fold skin sample collector peelable release panel, applying the adhesive patch to a skin sample, removing the used adhesive patch containing a skin sample and placing the used patch on the placement area sheet. In some instances, the placement area panel is a single placement area panel sheet. In some instances, the identity of the skin sample collected is indexed to the tri-fold skin sample collector or placement area panel sheet by using a barcode or printing patient information on the collector or panel sheet. In some instances, the indexed tri-fold skin sample collector or placement sheet is sent to a diagnostic lab for processing. In some instances, the used patch is configured to be stored on the placement panel for at least 1 week at temperatures between −80° C. and 25° C. In some embodiments, the used patch is configured to be stored on the placement area panel for at least 2 weeks, at least 3 weeks, at least 1 month, at least 2 months, at least 3 months, at least 4 months, at least 5 months, and at least 6 months at temperatures between −80° C. and 25° C. In some embodiments, the indexed tri-fold skin sample collector or placement sheet is sent to a diagnostic lab using UPS or FedEx.

In an exemplary embodiment, the patch stripping method further comprises preparing the skin sample prior to application of the adhesive patch. Preparation of the skin sample includes, but is not limited to, removing hairs on the skin surface, cleansing the skin surface and/or drying the skin surface. In some instances, the skin surface is cleansed with an antiseptic including, but not limited to, alcohols, quaternary ammonium compounds, peroxides, chlorhexidine, halogenated phenol derivatives and quinolone derivatives. In some instances, the alcohol is about 0 to about 20%, about 20 to about 40%, about 40 to about 60%, about 60 to about 80%, or about 80 to about 100% isopropyl alcohol. In some instances, the antiseptic is 70% isopropyl alcohol.

In some embodiments, the patch stripping method is used to collect a skin sample from the surfaces including, but not limited to, the face, head, neck, arm, chest, abdomen, back, leg, hand or foot. In some instances, the skin surface is not located on a mucous membrane. In some instances, the skin surface is not ulcerated or bleeding. In certain instances, the skin surface has not been previously biopsied. In certain instances, the skin surface is not located on the soles of the feet or palms.

The patch stripping method, devices, and systems described herein are useful for the collection of a skin sample from a skin lesion. A skin lesion is a part of the skin that has an appearance or growth different from the surrounding skin. In some instances, the skin lesion is pigmented. A pigmented lesion includes, but is not limited to, a mole, dark colored skin spot and a melanin containing skin area. In some embodiments, the skin lesion is from about 5 mm to about 16 mm in diameter. In some instances, the skin lesion is from about 5 mm to about 15 mm, from about 5 mm to about 14 mm, from about 5 mm to about 13 mm, from about 5 mm to about 12 mm, from about 5 mm to about 11 mm, from about 5 mm to about 10 mm, from about 5 mm to about 9 mm, from about 5 mm to about 8 mm, from about 5 mm to about 7 mm, from about 5 mm to about 6 mm, from about 6 mm to about 15 mm, from about 7 mm to about 15 mm, from about 8 mm to about 15 mm, from about 9 mm to about 15 mm, from about 10 mm to about 15 mm, from about 11 mm to about 15 mm, from about 12 mm to about 15 mm, from about 13 mm to about 15 mm, from about 14 mm to about 15 mm, from about 6 to about 14 mm, from about 7 to about 13 mm, from about 8 to about 12 mm and from about 9 to about 11 mm in diameter. In some embodiments, the skin lesion is from about 10 mm to about 20 mm, from about 20 mm to about 30 mm, from about 30 mm to about 40 mm, from about 40 mm to about 50 mm, from about 50 mm to about 60 mm, from about 60 mm to about 70 mm, from about 70 mm to about 80 mm, from about 80 mm to about 90 mm, and from about 90 mm to about 100 mm in diameter. In some instances, the diameter is the longest diameter of the skin lesion. In some instances, the diameter is the smallest diameter of the skin lesion.

The adhesive skin sample collection kit, in some embodiments, comprises at least one adhesive patch, a sample collector, and an instruction for use sheet. In an exemplary embodiment, the sample collector is a tri-fold skin sample collector comprising a peelable release panel comprising at least one adhesive patch, a placement area panel comprising a removable liner, and a clear panel. The tri-fold skin sample collector, in some instances, further comprises a barcode and/or an area for transcribing patient information. In some instances, the adhesive skin sample collection kit is configured to include a plurality of adhesive patches, including but not limited to 12, 11, 10, 9, 8, 7, 6, 5, 4, 3, 2, 1, from about 2 to about 8, from about 2 to about 7, from about 2 to about 6, from about 2 to about 4, from about 3 to about 6, from about 3 to about 8, from about 4 to about 10, from about 4 to about 8, from about 4 to about 6, from about 4 to about 5, from about 6 to about 10, from about 6 to about 8, or from about 4 to about 8. The instructions for use sheet provides the kit operator all of the necessary information for carrying out the patch stripping method. The instructions for use sheet preferably includes diagrams to illustrate the patch stripping method.

In some instances, the adhesive skin sample collection kit provides all the necessary components for performing the patch stripping method. In some embodiments, the adhesive skin sample collection kit includes a lab requisition form for providing patient information. In some instances, the kit further comprises accessory components. Accessory components include, but are not limited to, a marker, a resealable plastic bag, gloves and a cleansing reagent. The cleansing reagent includes, but is not limited to, an antiseptic such as isopropyl alcohol. In some instances, the components of the skin sample collection kit are provided in a cardboard box.

### Tissue Sampling and Cellular Material

The methods and devices provided herein, in certain embodiments, involve applying an adhesive or other similar patch to the skin in a manner so that an effective or sufficient amount of a tissue, such as a skin sample, adheres to the adhesive matrix of the adhesive patch. For example, the effective or sufficient amount of a skin sample is an amount that removably adheres to a material, such as the matrix or adhesive patch. The adhered skin sample, in certain embodiments, comprises cellular material including nucleic acids and proteins. In some instances, the nucleic acid is RNA or DNA. An effective amount of a skin sample contains an amount of cellular material sufficient for performing a diagnostic assay. In some instances, the diagnostic assay is performed using the cellular material isolated from the adhered skin sample on the used adhesive patch. In some instances, the diagnostic assay is performed on the cellular material adhered to the used adhesive patch. In some embodiments, an effect amount of a skin sample comprises an amount of RNA sufficient to perform a gene expression analysis. Sufficient amounts of RNA includes, but not limited to, picogram, nanogram, and microgram quantities.

In still further or additional embodiments, the adhered skin sample comprises cellular material including nucleic acids such as RNA or DNA, or a polypeptide such as a protein, in an amount that is at least about 1 picogram. In some embodiments, the amount of cellular material is no more than about 1 nanogram. In further or additional embodiments, the amount of cellular material is no more than about 1 microgram. In still further or additional embodiments, the amount of cellular material is no more than about 1 gram.

In further or additional embodiments, the amount of cellular material is from about 1 picogram to about 1 gram. In further or additional embodiments, the cellular material comprises an amount that is from about 50 microgram to about 1 gram, from about 100 picograms to about 500 micrograms, from about 500 picograms to about 100 micrograms, from about 750 picograms to about 1 microgram, from about 1 nanogram to about 750 nanograms, or from about 1 nanogram to about 500 nanograms.

In further or additional embodiments, the amount of cellular material, including nucleic acids such as RNA or DNA, or a polypeptide such as a protein, comprises an amount that is from about 50 microgram to about 500 microgram, from about 100 microgram to about 450 microgram, from about 100 microgram to about 350 microgram, from about 100 microgram to about 300 microgram, from about 120 microgram to about 250 microgram, from about 150 microgram to about 200 microgram, from about 500 nanograms to about 5 nanograms, or from about 400 nanograms to about 10 nanograms, or from about 200 nanograms to about 15 nanograms, or from about 100 nanograms to about 20 nanograms, or from about 50 nanograms to about 10 nanograms, or from about 50 nanograms to about 25 nanograms.

In further or additional embodiments, the amount of cellular material, including nucleic acids such as RNA or DNA, or a polypeptide such as a protein, is less than about 1 gram, is less than about 500 micrograms, is less than about 490 micrograms, is less than about 480 micrograms, is less than about 470 micrograms, is less than about 460 micrograms, is less than about 450 micrograms, is less than about 440 micrograms, is less than about 430 micrograms, is less than about 420 micrograms, is less than about 410 micrograms, is less than about 400 micrograms, is less than about 390 micrograms, is less than about 380 micrograms, is less than about 370 micrograms, is less than about 360 micrograms, is less than about 350 micrograms, is less than about 340 micrograms, is less than about 330 micrograms, is less than about 320 micrograms, is less than about 310 micrograms, is less than about 300 micrograms, is less than about 290 micrograms, is less than about 280 micrograms, is less than about 270 micrograms, is less than about 260 micrograms, is less than about 250 micrograms, is less than about 240 micrograms, is less than about 230 micrograms, is less than about 220 micrograms, is less than about 210 micrograms, is less than about 200 micrograms, is less than about 190 micrograms, is less than about 180 micrograms, is less than about 170 micrograms, is less than about 160 micrograms, is less than about 150 micrograms, is less than about 140 micrograms, is less than about 130 micrograms, is less than about 120 micrograms, is less than about 110 micrograms, is less than about 100 micrograms, is less than about 90 micrograms, is less than about 80 micrograms, is less than about 70 micrograms, is less than about 60 micrograms, is less than about 50 micrograms, is less than about 20 micrograms, is less than about 10 micrograms, is less than about 5 micrograms, is less than about 1 microgram, is less than about 750 nanograms, is less than about 500 nanograms, is less than about 250 nanograms, is less than about 150 nanograms, is less than about 100 nanograms, is less than about 50 nanograms, is less than about 25 nanograms, is less than about 15 nanograms, is less than about 1 nanogram, is less than about 750 picograms, is less than about 500 picograms, is less than about 250 picograms, is less than about 100 picograms, is less than about 50 picograms, is less than about 25 picograms, is less than about 15 picograms, or is less than about 1 picogram.

In some embodiments, isolated RNA from a collected skin sample is reverse transcribed into cDNA, for example for amplification by PCR to enrich for target genes. The expression levels of these target genes are quantified by quantitative PCR in a gene expression test. In some instances, in combination with quantitative PCR, a software program performed on a computer is utilized to quantify RNA isolated from the collected skin sample. In some instances, a software program or module is utilized to relate a quantity of RNA from a skin sample to a gene expression signature, wherein the gene expression signature is associated with a disease such as skin cancer. In some embodiments, a software program or module scores a sample based on gene expression levels. In some embodiments, the sample score is compared with a reference sample score to determine if there is a statistical significance between the gene expression signature and a disease.

### Computer Program

The methods, software, media, and systems disclosed herein comprise at least one computer processor, or use of the same. In some instances, the computer processor comprises a computer program. In some instances, a computer program includes a sequence of instructions, executable in the digital processing device's CPU, written to perform a specified task. In some instances, computer readable instructions are implemented as program modules, such as functions, features, Application Programming Interfaces (APIs), data structures, and the like, that perform particular tasks or implement particular abstract data types. In light of the disclosure provided herein, those of skill in the art will recognize that a computer program, in some embodiments, are written in various versions of various languages.

The functionality of the computer readable instructions, in certain embodiments, are combined or distributed as desired in various environments. In some instances, a computer program comprises one sequence of instructions. In some instances, a computer program comprises a plurality of sequences of instructions. In some instances, a computer program is provided from one location. In some instances, a computer program is provided from a plurality of locations. In some instances, a computer program includes one or more software modules. In some instances, a computer program includes, in part or in whole, one or more web applications, one or more mobile applications, one or more standalone applications, one or more web browser plug-ins, extensions, add-ins, or add-ons, or combinations thereof

### Web Application

In some instances, a computer program includes a web application. In light of the disclosure provided herein, those of skill in the art will recognize that a web application, in certain embodiments, utilizes one or more software frameworks and one or more database systems. In some instances, a web application is created upon a software framework such as Microsoft® .NET or Ruby on Rails (RoR). In some instances, a web application utilizes one or more database systems including, by way of non-limiting examples, relational, non-relational, feature oriented, associative, and XML database systems. Suitable relational database systems includes, by way of non-limiting examples, Microsoft© SQL Server, mySQL™, and Oracle®. Those of skill in the art will also recognize that a web application, in certain embodiments, is written in one or more versions of one or more languages. In some instances, a web application is written in one or more markup languages, presentation definition languages, client-side scripting languages, server-side coding languages, database query languages, or combinations thereof. In some instances, a web application is written to some extent in a markup language such as Hypertext Markup Language (HTML), Extensible Hypertext Markup Language (XHTML), or eXtensible Markup Language (XML). In some instances, a web application is written to some extent in a presentation definition language such as Cascading Style Sheets (CSS). In some instances, a web application is written to some extent in a client-side scripting language such as Asynchronous Javascript and XML (AJAX), Flash® Actionscript, Javascript, or Silverlight®. In some instances, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some instances, a web application is written to some extent in a database query language such as Structured Query Language (SQL). In some instances, a web application integrates enterprise server products such as IBM® Lotus Domino®. In some instances, a web application includes a media player element. In some instances, a media player element utilizes one or more of many suitable multimedia technologies including, by way of non-limiting examples, Adobe® Flash®, HTML 5, Apple® QuickTime®, Microsoft® Silverlight®, Java™, and Unity®.

### Mobile Application

In some instances, a computer program includes a mobile application provided to a mobile digital processing device. In some instances, the mobile application is provided to a mobile digital processing device at the time it is manufactured. In some instances, the mobile application is provided to a mobile digital processing device via the computer network described herein.

In some instances, the mobile application is created by techniques known to those of skill in the art using hardware, languages, and development environments known to the art. Those of skill in the art will recognize that mobile applications, in certain embodiments, are written in several languages. Suitable programming languages include, by way of non-limiting examples, C, C++, C#, Featureive-C, Java™, Javascript, Pascal, Feature Pascal, Python™, Ruby, VB.NET, WML, and XHTML/HTML with or without CSS, or combinations thereof.

Suitable mobile application development environments, in some instances, are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. In some instances, other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some instances, a computer program includes a standalone application, which is a program that is run as an independent computer process, not an add-on to an existing process, e.g., not a plug-in. Those of skill in the art will recognize that standalone applications are often compiled. In some instances, a compiler is a computer program(s) that transforms source code written in a programming language into binary feature code such as assembly language or machine code. Suitable compiled programming languages include, by way of non-limiting examples, C, C++, Featureive-C, COBOL, Delphi, Eiffel, Java™, Lisp, Python™, Visual Basic, and VB NET, or combinations thereof. Compilation are often performed, at least in part, to create an executable program. In some instances, a computer program includes one or more executable complied applications.

### Web Browser Plug-In

In some instances, a computer program includes a web browser plug-in. In computing, a plug-in, in some instances, is one or more software components that add specific functionality to a larger software application. In some instances, makers of software applications support plug-ins to enable third-party developers to create abilities which extend an application, to support easily adding new features, and to reduce the size of an application. In some instances, when supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash© Player, Microsoft© Silverlight®, and Apple® QuickTime®. In some instances, the toolbar comprises one or more web browser extensions, add-ins, or add-ons. In some instances, the toolbar comprises one or more explorer bars, tool bands, or desk bands.

In view of the disclosure provided herein, those of skill in the art will recognize that several plug-in frameworks, in some instances, are available that enable development of plug-ins in various programming languages, including, by way of non-limiting examples, C++, Delphi, Java™, PHP, Python™, and VB .NET, or combinations thereof.

In some instances, web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some instances, web browser is a mobile web browser. In some instances, the mobile web browsers (also called microbrowsers, mini-browsers, and wireless browsers) are designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Software Modules

The medium, method, and system disclosed herein comprise one or more softwares, servers, and database modules, or use of the same. In view of the disclosure provided herein, software modules, in certain embodiments, are created by techniques known to those of skill in the art using machines, software, and languages known to the art. The software modules disclosed herein, in certain embodiments, are implemented in a multitude of ways. In some instances, a software module comprises a file, a section of code, a programming feature, a programming structure, or combinations thereof. In some instances, a software module comprises a plurality of files, a plurality of sections of code, a plurality of programming features, a plurality of programming structures, or combinations thereof. In some instances, the one or more software modules comprises, by way of non-limiting examples, a web application, a mobile application, and a standalone application. In some instances, software modules are in one computer program or application. In some instances, software modules are in more than one computer program or application. In some instances, software modules are hosted on one machine. In some instances, software modules are hosted on more than one machine. In some instances, software modules are hosted on cloud computing platforms. In some instances, software modules are hosted on one or more machines in one location. In some instances, software modules are hosted on one or more machines in more than one location.

### Databases

The medium, method, and system disclosed herein comprise one or more databases, or use of the same. In view of the disclosure provided herein, those of skill in the art will recognize that many databases, in certain embodiments, are suitable for storage and retrieval of geologic profile, operator activities, division of interest, and/or contact information of royalty owners. Suitable databases include, by way of non-limiting examples, relational databases, non-relational databases, feature oriented databases, feature databases, entity-relationship model databases, associative databases, and XML databases. In some instances, a database is internet-based. In some instances, a database is web-based. In some instances, a database is cloud computing-based. In some instances, a database is based on one or more local computer storage devices.

### Definitions

Throughout this disclosure, various embodiments are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

As used herein, the terms “individual(s)”, “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

As disclosed herein, a gene of interest described above comprises MMP1 (also known as matrix metallopeptidase 1, interstitial collagenase, fibroblast collagenase, or CLGN). In some instances, MMP1 has Gene ID: 4312.

As disclosed herein, a gene of interest described above comprises S100A7 (also known as S100 calcium binding protein A7, PSOR1, or psoriasin 1). In some instances, S100A7 has Gene ID: 6278.

As disclosed herein, a gene of interest described above comprises CMPK2 (also known as cytidine/uridine monophosphate kinase 2, nucleoside-diphosphate kinase, thymidine monophosphate kinase 2, TMPK2, TYKi, or NDK). In some instances, CMPK2 has Gene ID: 129607.

As disclosed herein, a gene of interest described above comprises IRF7 (also known as interferon regulatory factor 7 or IMD39). In some instances, IRF7 has Gene ID: 3665.

As disclosed herein, a gene of interest described above comprises IGFL1 (also known as IGF like family member 1, APRG644, or UNQ644). In some instances, IGFL1 has Gene ID: 374918.

As disclosed herein, a gene of interest described above comprises CXCL1 (also known as C-X-C motif chemokine ligand 1, GRO1 oncogene (melanoma growth stimulating activity, alpha), growth-regulated alpha protein, fibroblast secretory protein, NAP-3, SCYB1, or GRO1). In some instances, CXCL1 has Gene ID: 2919.

As disclosed herein, a gene of interest described above comprises UPP1 (also known as Uridine phosphorylase 1, UrdPase 1, UP, UPASE, or UPP). In some instances, UPP1 has Gene ID: 7378.

As disclosed herein, a gene of interest described above comprises DEFB4A (also known as defensin beta 4A, skin-antimicrobial peptide 1, DEFB102, or HBD-2). In some instances, DEFB4A has Gene ID: 1673.

As disclosed herein, a gene of interest described above comprises FOS (also known as Fos proto-oncogen, AP-1 transcription factor subunit; FBJ murine osteosarcoma viral oncogene homolog; GO/Gi switch regulatory protein 7; C-FOS; GOS7; or P55). In some instances, FOS has Gene ID: 2353.

As disclosed herein, a gene of interest described above comprises OAS3 (also known as 2′-5′-oligoacehylate synthetase 3, P100 OA, or P100). In some instances, OAS3 has Gene ID: 4940.

As disclosed herein, a gene of interest described above comprises SCD5 (also known as stearoyl-CoA desaturase 5, acyl-CoA-desaturase 4, SCD4, SCD2, or FADS4). In some instances, SCD5 has Gene ID: 79966.

As disclosed herein, a gene of interest described above comprises RTP4 (also known as receptor transporter protein 4, 28 KDa interferon-responsive protein, 3CxxC-type zinc finger protein 4, or IFRG28). In some instances, RTP4 has Gene ID: 64108.

As disclosed herein, a gene of interest described above comprises VEGFA (also known as vascular endothelial growth factor A or VPF). In some instances, VEGFA has Gene ID: 7422.

As disclosed herein, a gene of interest described above comprises COL5A2 (also known as collagen type V alpha 2 chain, AB collagen, or EDSC). In some instances, COL5A2 has Gene ID: 1290.

As disclosed herein, a gene of interest described above comprises IL24 (also known as ST16, MDA7, FISP, or MOBS). In some instances, IL24 has Gene ID: 11009.

As disclosed herein, a gene of interest described above comprises AADACL2 (also known as arylacetamide deacetylase-like 2). In some instances, AADACL2 has Gene ID: 344752.

As disclosed herein, a gene of interest described above comprises PTCH1 (also known as patched 1 or BCNS). In some instances, PTCH1 has Gene ID: 5727.

As disclosed herein, a gene of interest described above comprises CD68 (also known as scavenger receptor class D, member 1; macrophage antigen CD68, GP110, or LAMP4). In some instances, CD68 has Gene ID: 968.

As disclosed herein, a gene of interest described above comprises PRKACA (also known as protein kinase CAMP-activated catalytic subunit alpha or PPNAD4). In some instances, PRKACA has Gene ID: 5566.

As disclosed herein, a gene of interest described above comprises SPP1 (also known as secreted phosphoprotein 1, osteopontin, nephropontin, BNSP, OPN, or BSPI). In some instances, SPP1 has Gene ID: 6696.

### EXAMPLES

The following examples are given for the purpose of illustrating various embodiments of the disclosure and are not meant to limit the present disclosure in any fashion. The present examples, along with the methods described herein are presently representative of preferred embodiments, are exemplary, and are not intended as limitations on the scope of the disclosure. Changes therein and other uses which are encompassed within the spirit of the disclosure as defined by the scope of the claims will occur to those skilled in the art.

### Example 1

### Non-Invasive Gene Expression Assay for Cutaneous Basal Cell and Squamous Cell Carcinoma

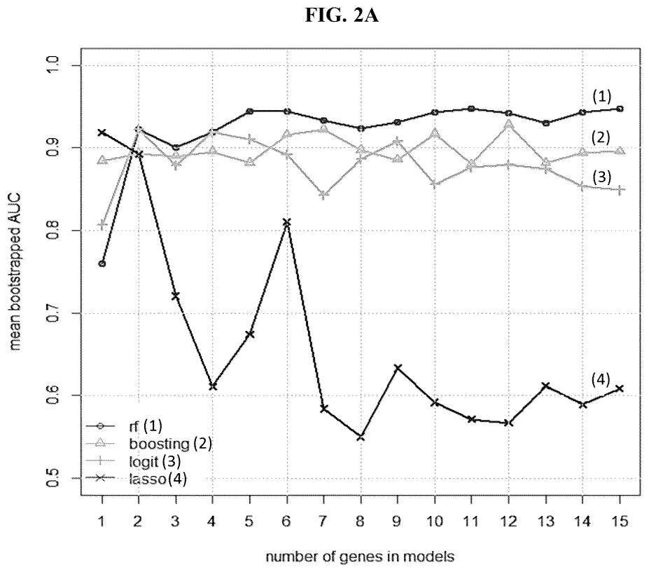

A qPCR gene expression test was designed using identified target genes. The identified target genes were evaluated in prospectively collected basal cell carcinoma (BCC), squamous cell carcinoma (SCC), actinic keratosis (AK), seborrheic keratosis (SK), and normal samples obtained from subjects via non-invasive adhesive patch biopsies. Total RNA were extracted from these samples and quantified with real time quantitative PCR (qPCR, TaqMan qPCR) based on a house keeping gene (beta-actin). Cycle threshold (Ct) values from qPCR analyses were used to demonstrate changes in target gene expression. Algorithms were developed (FIGS. 2A-2B), trained, and subjected to primary validation in histopathologically confirmed samples (n=160 cases). The variables for analysis included interactions among MMP1, S100A7, CMPK2, IRF7, IGFL1, CXCL1, UPP1, DEFB4A, FOS, OAS3, SCD5, RTP4, and VEGFA genes as seen in Table 1 and Table 2.

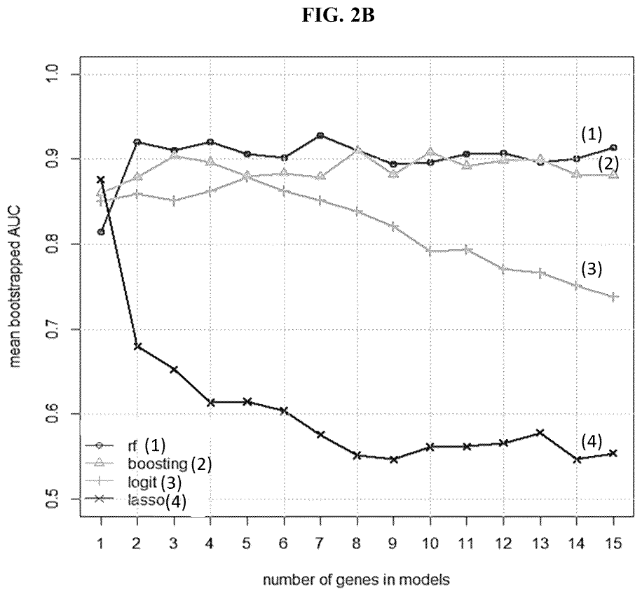

Gene expression changes of the target genes in the samples (cancer or non-cancer) were analyzed and compared with target-specific qPCR that received a normalized input of total RNA from each sample. Cancer samples and non-cancer samples were differentiated on the different gene expression patterns of these target genes. Using a 13-target gene panel (MMP1, S100A7, CMPK2, IRF7, IGFL1, CXCL1, UPP1, DEFB4A, FOS, OAS3, SCD5, RTP4, and VEGFA), BCC and SCC samples were differentiated from AK and other non-cancerous skin lesions of similar appearance with a sensitivity of 91% (95% CI 86%-95%) and a specificity of 87% (95% CI 80%-92%) based on 160 non-invasively collected adhesive patch skin biopsies (p<0.001) when employing the best performing random forest (rf) model. An area under the curve (AUC) value of 0.95 was observed (FIG. 3A) when comparing BCC and SCC to AK, SK, and NML samples. AUC values were also determined following comparison of BCC and SCC with SK samples (FIG. 3B).

This example shows that non-invasive gene expression analysis differentiates primary cutaneous BCC and SCC samples from benign and precursor lesions such as AK with high sensitivity and specificity.

### Example 2

### Non-Invasive Gene Expression Assay Utilizing a 9-Target Gene Panel

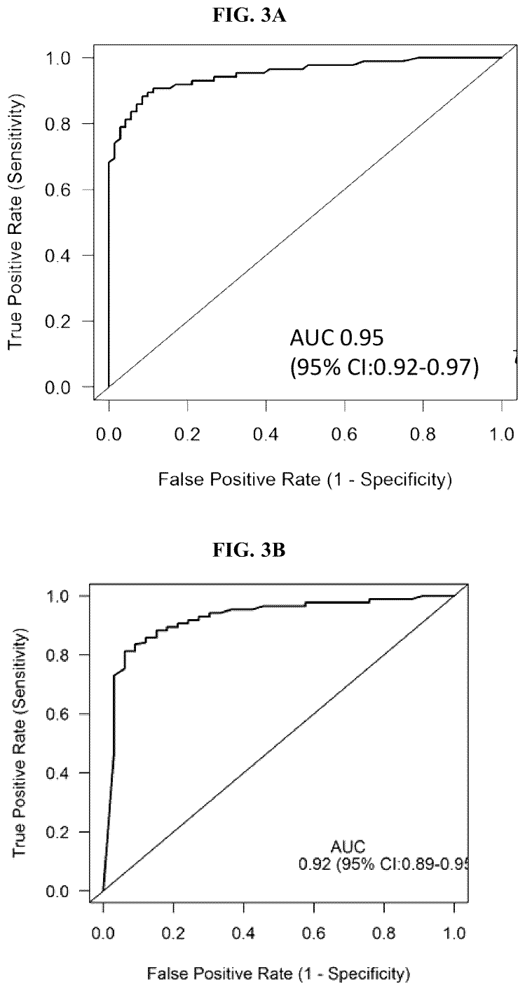

A qPCR gene expression test was designed using identified target genes. The identified target genes were evaluated in prospectively collected basal cell carcinoma (BCC), squamous cell carcinoma (SCC), actinic keratosis (AK), seborrheic keratosis (SK), and normal samples obtained from subjects via non-invasive adhesive patch biopsies. Total RNA were extracted from these samples and quantified with real time quantitative PCR (qPCR, TaqMan qPCR) based on a house keeping gene (beta-actin). Cycle threshold (Ct) values from qPCR analyses were used to demonstrate changes in target gene expression. Algorithms were developed (FIG. 4), trained, and subjected to primary validation in histopathologically confirmed samples. The variables for analysis included interactions among IGFL1, MMP1, COL5A2, IL24, AADACL2, PTCH1, CD68, PRKACA, and SPP1 as seen in Table 3.

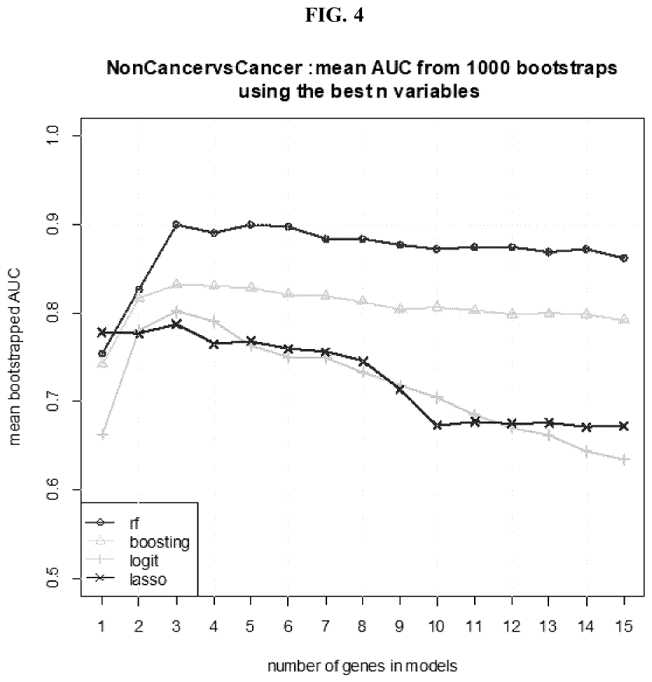

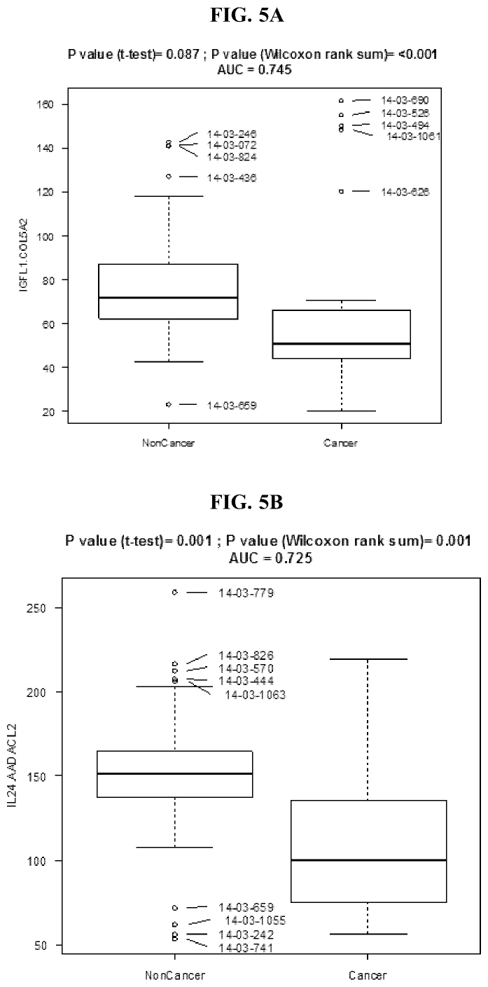

Gene expression changes of the target genes in the samples (cancer or non-cancer) were analyzed and compared with target-specific qPCR that received a normalized input of total RNA from each sample. Cancer samples and non-cancer samples were differentiated on the different gene expression patterns of these target genes. Using a 9-target gene panel (IGFL1, MMP1, COL5A2, IL24, AADACL2, PTCH1, CD68, PRKACA, and SPP1) (FIGS. 5A-5E) BCC and SCC samples were differentiated from AK and other non-cancerous skin lesions of similar appearance with a sensitivity of about 90% and a specificity of from about 70% to 75% (FIGS. 6A-6C).

This example shows that non-invasive gene expression analysis differentiates BCC and SCC samples from benign and precursor lesions such as AK with high sensitivity and specificity.

### Example 3

### Biplex PCR Amplifications of CDKN2A and TERT Exons

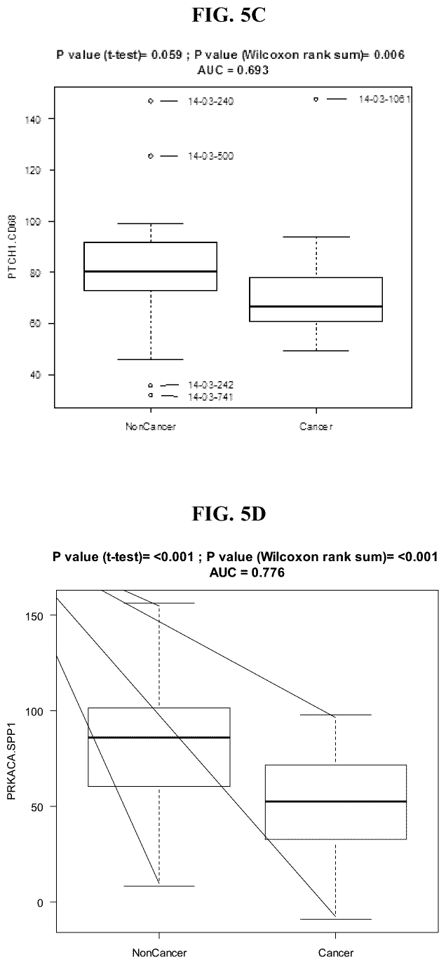

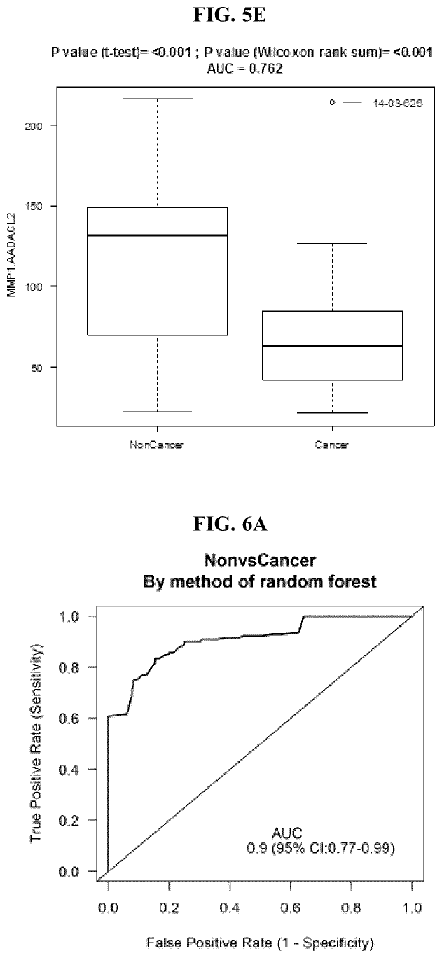

FIG. 7A shows an exemplary biplex PCR amplification of CDKN2A and TERT exons. FIG. 7B shows an exemplary Sanger sequencing of CDKN2A and TERT wild-type sequences. The locations of the CDKN2A mutations are further denoted in FIG. 7B. The PCR reaction was prepared based on the reaction setup illustrated in Table 5.

| | Vol (μl) |
|---|---|
| H2O | 3.7 |
| AccuStart II GelTrack PCR SuperMix (2x) | 12.5 |
| *Forward Primer (5 uM) (CDKN2A) | 0.5 |
| *Reverse Primer (5 uM) (CDKN2A) | 0.5 |
| *Forward Primer (5 uM) (TERT) | 1.0 |
| *Reverse Primer (5 uM) (TERT) | 1.0 |
| DMSO (3%) | 0.8 |
| gDNA, 100 pg total input | 5.0 |
| Total | 25.0 |

### Example 4

### PCR Amplification of PTCH1

About 305 BCC samples and about 25 SCC samples were processed for detection mutations in PTCH1. About 167 BCC PTCH1 mutations and about 14 SCC PTCH1 mutations were detected from the tested samples.

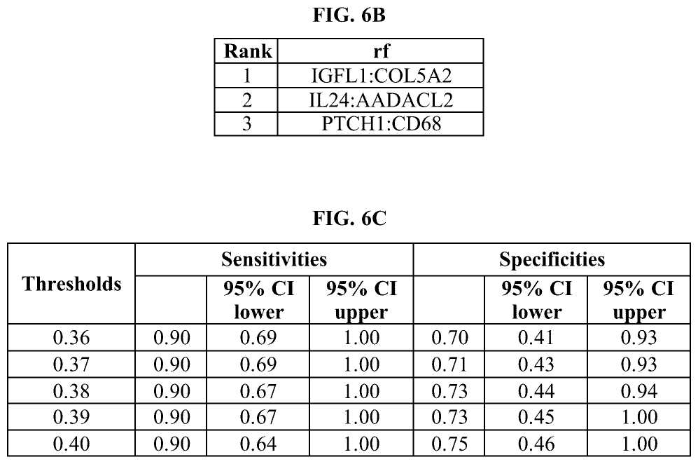

FIGS. 8A and 8B illustrate the mutation analysis of exemplary exons in PTCH1. FIG. 8A shows the number of mutations detected with respect to each exon from BCC, SCC, and AK. FIG. 8B shows the number of exons detected and the number of mutations detected within each exon in the tested BCC samples.

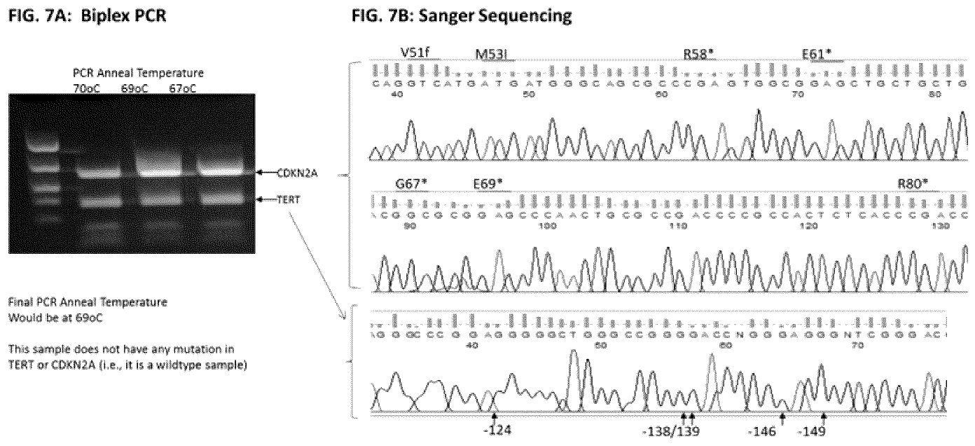

FIG. 9 illustrates the PCR amplification of exon 14, exon 15, and exon 23.

The PCR reaction was prepared based on the reaction setup illustrated in Table 6.

| | Vol (μl) |
|---|---|
| H2O | 5.50 |
| AccuStart II GelTrack PCR SuperMix (2x) | 12.50 |
| Ptch 1 Forward (5 uM) | 1.00 |
| Ptch 1 Reverse (5 uM) | 1.00 |
| gDNA, 100 pg/ul | 5.00 |
| Total | 25.00 |

### Example 5

### PCR Amplification of TP53

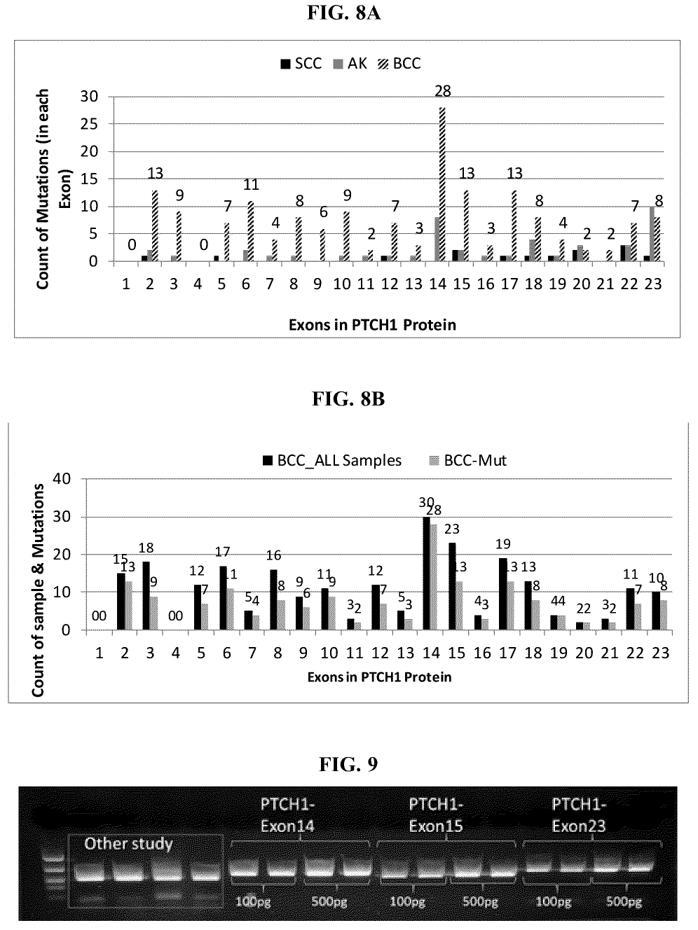

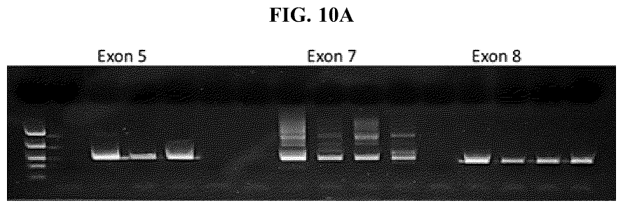

FIG. 10A illustrates the PCR amplification of exon 5, exon 7, and exon 8. FIG. 10B shows the mutation location within each exon sequence. The PCR reaction was prepared based on the reaction setup illustrated in Table 7.

| | Vol (μl) |
|---|---|
| H2O | 5.50 |
| AccuStart II GelTrack PCR SuperMix (2x) | 12.50 |
| TP53 Ex 5, 7, 8 F (5 uM) | 1.00 |
| TP53 Ex 5, 7, 8 R (5 uM) | 1.00 |
| gDNA, 100 pg/ul | 5.00 |
| Total | 25.00 |

### Example 6

### Co-Isolation of RNA and DNA Using Silica-Coated Magnetic Beads

Skin samples were collected with adhesive patches from forehead of adult volunteers, each patch was cut by half, and each half was used side-by-side for either cell lysis and nucleic acid extraction with in-house procedure or for cell lysis and nucleic acid extraction with a commercial test kit. The 2 halves were randomly split and used in each comparison test.

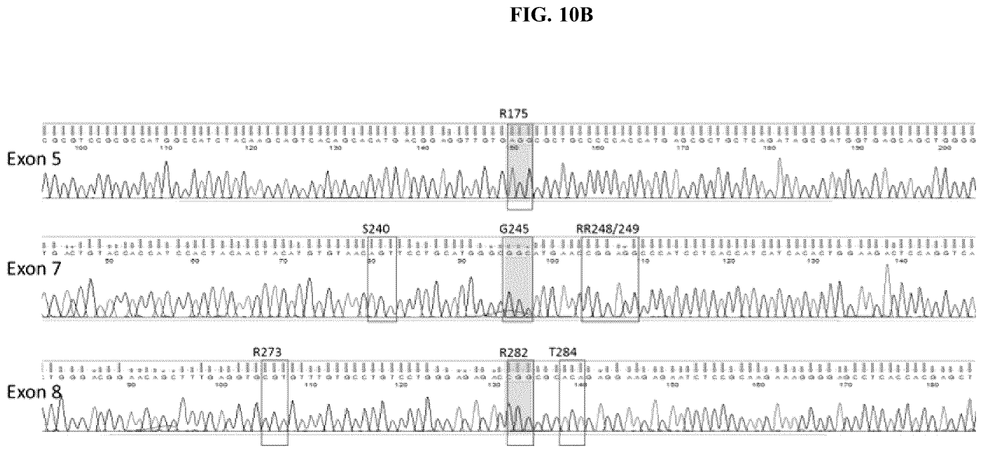

The percentage of RNA and DNA recovery was tested utilizing a mixture of silica-coated magnetic beads (AccuBead from Bioneer) (referenced as DT MB in the figure) and magnetic beads from Zymo Research (referenced as Zymo MB in the figure). After incubation, the magnetic beads in these tubes were washed either in a wash buffer prepared in-house or in a wash buffer from Zymo Research, and finally all samples were eluted in an in-house elution buffer. Total RNA and gDNA from all eluents were shown in FIG. 11. As illustrated in FIG. 11, the DT MB provided higher recovery of total RNA while the Zymo MB provided higher recovery of gDNA.

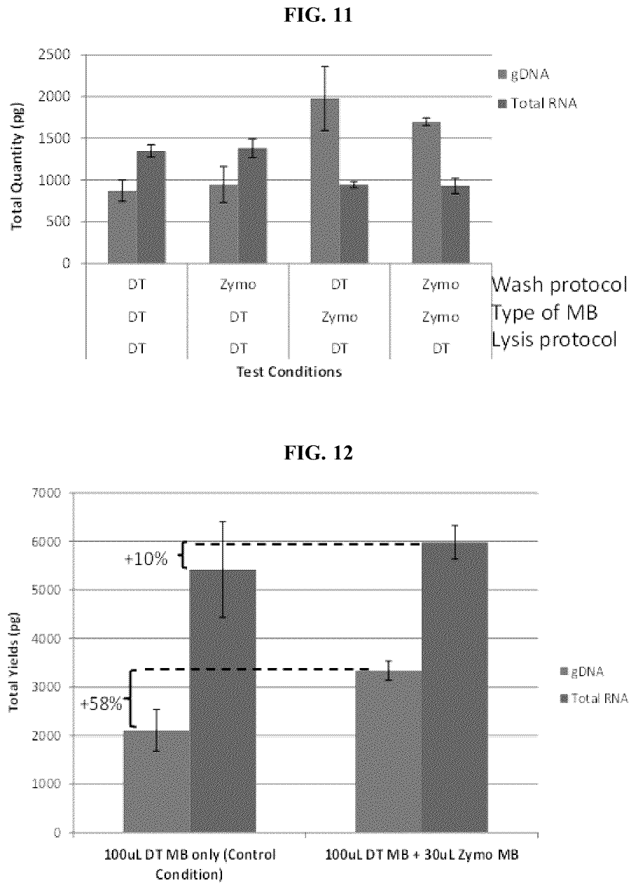

Based on the results from FIG. 11, different volume ratios of the DT MB and Zymo MB were tested. FIG. 12 illustrates gDNA and total RNA extraction utilizing a 100 μL DT MB:30 μL Zymo MB ratio compared to the control, which contains 100 μL of DT MB.

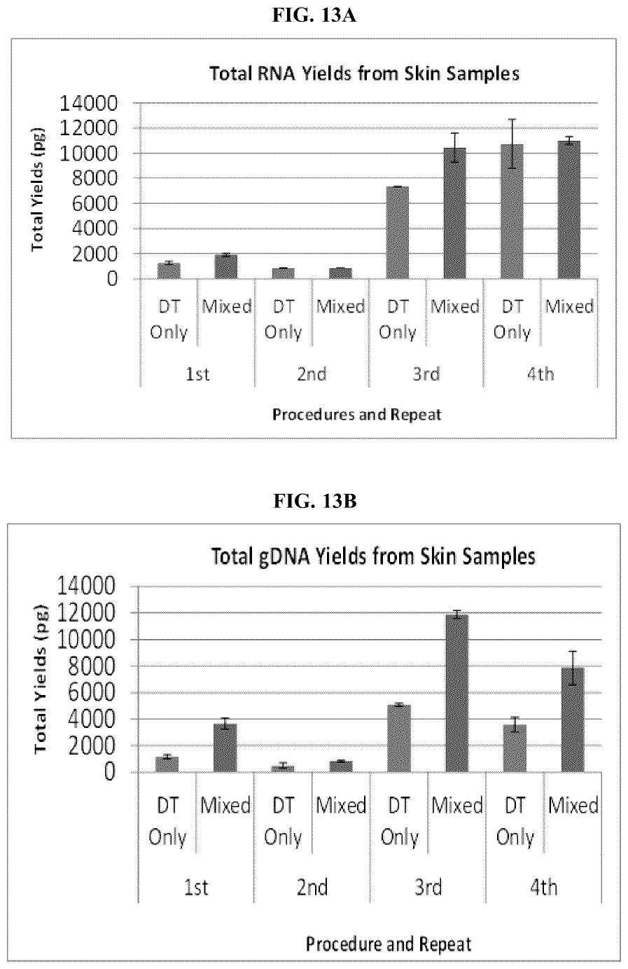

Four repeat skin sample extraction runs were made to compare side-by-side the procedure utilizing the mixed beads to the procedure utilizing single source beads. Forehead skin samples were collected with adhesive patches from adult volunteers, lysed in bulk volume with lysis buffer and then split to tubes to incubate with either the 2 types of beads (‘Mixed’) or the single type of bead (‘DT Only’). The rest steps (bead washing and nucleic acid elution) followed. In each run, the DT Only and the Mix use the same sources of skin samples but the 4 runs were done on different days on skin samples collected from different adult volunteers. FIG. 13A-FIG. 13B show 4 exemplary follow-up studies comparing the recovery of RNA and gDNA between DT MB, Zymo MB, and a mixture of DT MB and Zymo MB.

Gene expressions of 12 RNA samples isolated from forehead skins collected on adhesive patches from 6 adult volunteers were analyzed. Skin samples from the subjects were lysed and split for nucleic acid extraction. Beta-actin (control) and genes from the 9-target gene panel described in Example 2 were detected. Melanoma marker genes such as LINC and PRAME were not detected.

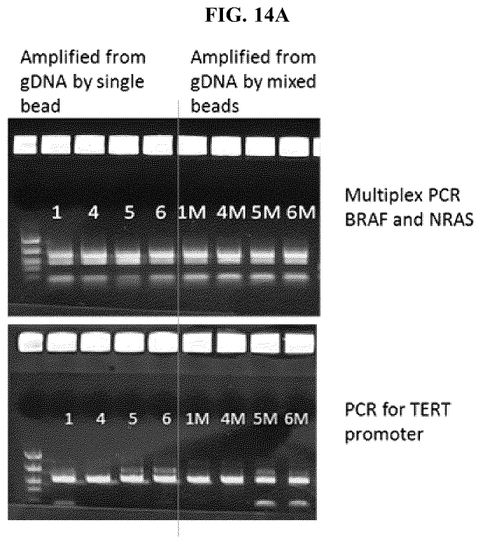

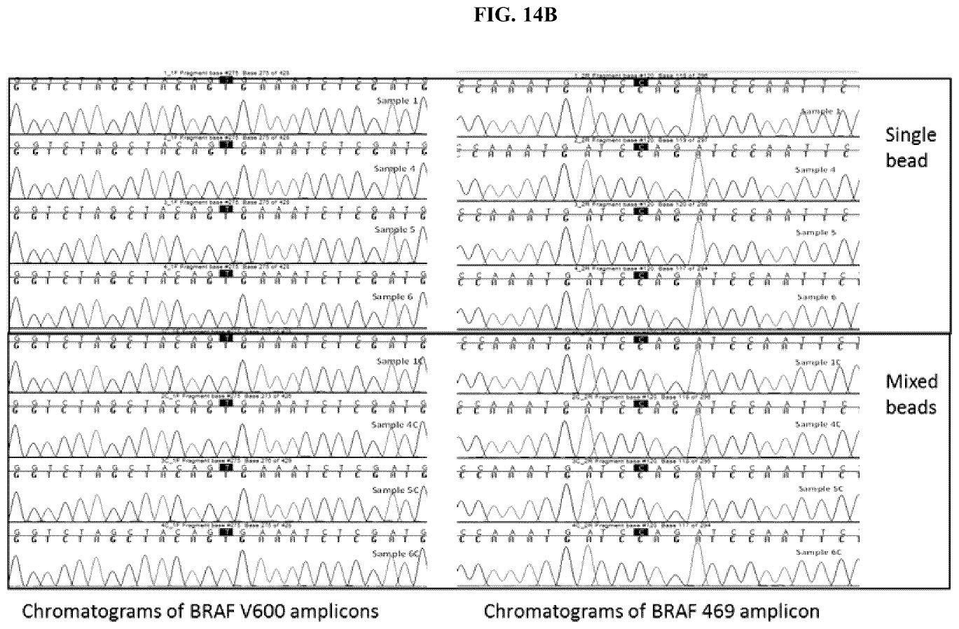

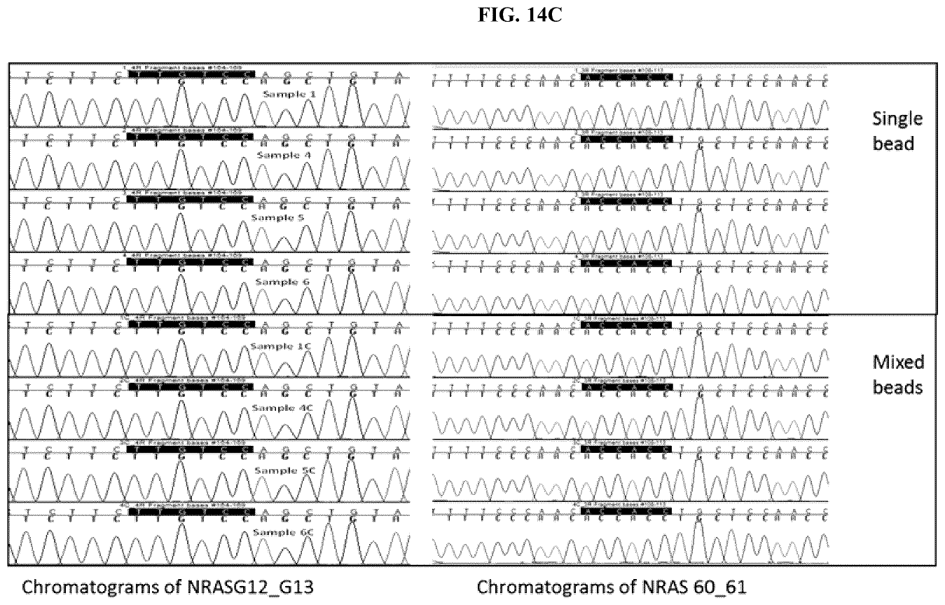

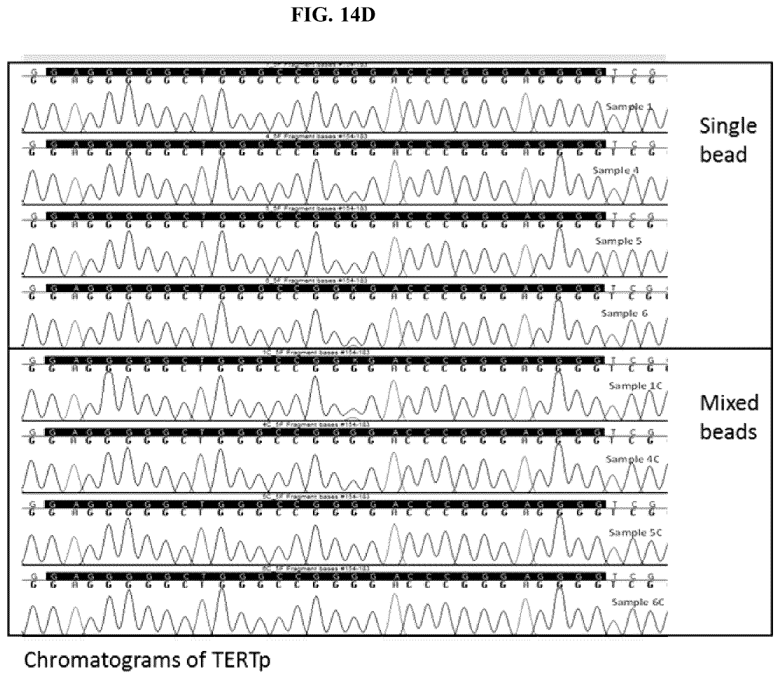

gDNA in the isolated NA from the same samples used for gene expression analysis were also tested for mutation. FIG. 14A shows PCR amplification of multiplex BRAF and NRAS amplicons (top) and TERT amplicon (bottom) from gDNA in these isolated NA samples. Samples 1, 4, 5 and 6 were from gDNA isolated by the old procedure (with DT only bead) and samples 1M, 4M, 5M and 6M were from gDNA isolated by the new procedure (with mixed beads). All samples were amplified well in PCR.

FIG. 14B-FIG. 14C show chromatograms of Sanger sequencing on amplicons for BRAF V600, and G469, and NRAS G12,13 and NRAS Q61 from PCR. FIG. 14D shows chromatogram of Sanger sequencing on TERT amplicon from PCR reaction.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A method of detecting gene expression levels of at least two of IGFL1, MMP1, COL5A2, IL24, AADACL2, PTCH1, CD68, PRKACA, and SPP1 in a subject in need thereof, comprising:
(a) isolating nucleic acids from a biological sample obtained from the subject; and
(b) detecting the expression levels of the at least two of IGFL1, MMP1, COL5A2, IL24, AADACL2, PTCH1, CD68, PRKACA, and SPP1, by contacting the isolated nucleic acids with a set of probes that recognizes the at least two of IGFL1, MMP1, COL5A2, IL24, AADACL2, PTCH1, CD68, PRKACA, and SPP1, and detects binding between the at least two of IGFL1, MMP1, COL5A2, IL24, AADACL2, PTCH1, CD68, PRKACA, and SPP1 and the set of probes.

2. The method of claim 1, wherein the set of probes recognizes:
either IGFL1 or MMP1 in combination with COL5A2, IL24, AADACL2, PTCH1, CD68, PRKACA, or SPP1, or a combination thereof;
IGFL1 and MMP1 in combination with COL5A2, IL24, AADACL2, PTCH1, CD68, PRKACA, or SPP1, or a combination thereof; or
IGFL1, MMP1, COL5A2, IL24, AADACL2, PTCH1, CD68, PRKACA, and SPP1.

3. The method of claim 1, further comprising detecting a mutational change of TERT, CDKN2A, TP53, or PTCH1, or a combination thereof.

4. The method of claim 3, wherein a mutation in TP53 translates to amino acid positions in TP53 selected from: R175, S240, G245, R248, R249, R273, R282, or T284, wherein the numberings of the amino acid residues correspond to SEQ ID NO: 1.

5. The method of claim 3, wherein a mutation in TP53 is in exon 1, 2, 3, 4, 5, 6, 7, 8, 9, 10, 11, or a combination thereof.

6. The method of claim 3, wherein a mutation in TP53 is in exon 5, 7, 8, or a combination thereof.

7. The method of claim 3, wherein a mutation in PTCH1 is in exon 1, 2, 3, 4, 5, 6, 7, 8, 9, 10, 11, 12, 13, 14, 15, 16, 17, 18, 19, 20, 21, 22, 23, 24, or a combination thereof of PTCH1.

8. The method of claim 3, wherein a mutation in PTCH1 is in exon 14, 15, 17, 23, or a combination thereof.

9. The method of claim 3, wherein a mutation in CDKN2A is in exon 1, 2, 3, 4, 5, 6, 7, 8, or a combination thereof.

10. The method of claim 3, wherein a mutation in CDKN2A translates to amino acid positions in CDKN2A selected from V51, M53, R58, E61, G67, E69, or R80, wherein the numbering of the amino acid residues correspond to SEQ ID NO: 5.

11. The method of claim 3, wherein a mutation in TERT is in exon 1, 2, 3, 4, 5, 6, 7, 8, 9, 10, 11, 12, 13, 14, 15, 16, or a combination thereof.

12. The method of claim 3, wherein a mutation in TERT is in a promoter region of TERT.

13. The method of claim 3, wherein the mutational change comprises at least 1.5×, 2×, 3×, 4×, 5×, 6×, 7×, 8×, 9×, 10×, 11×, or 12× more mutations in TERT CDKN2A, TP53, PTCH1, or a combination thereof, compared to a normal biological sample.

14. The method of claim 3, wherein the mutational change comprises at least 10%, 15%, 20%, 25%, 30%, 35%, 40%, 45%, 50%, 55%, 60%, 65%, 70%, 75%, or 80% more mutations in TERT, CDKN2A, TP53, PTCH1, or a combination thereof, compared to a normal biological sample.

15. The method of claim 1, wherein the subject is suspected of having a non-melanoma skin cancer, optionally BCC or SCC.

16. The method of claim 1, wherein the biological sample comprises a skin sample, optionally comprising keratinocytes, melanocytes, basal cells, T-cells, or dendritic cells.

17. The method of claim 16, wherein the skin sample is obtained by applying a plurality of adhesive patches to the skin sample in a manner sufficient to adhere the skin sample to the adhesive patch, and removing the adhesive patch from the skin in a manner sufficient to retain the adhered skin sample to the adhesive patch.

18. The method of claim 1, wherein isolating the nucleic acids comprises using a plurality of silica-coated beads, optionally a plurality of silica-coated magnetic beads.

19. The method of claim 1, wherein the nucleic acids comprise RNA, DNA, or a combination thereof.

20. A method of detecting gene expression levels and mutational changes from a skin sample, comprising:
a) isolating nucleic acids from the skin sample; and
b) detecting the expression levels of one or more genes selected from: IGFL1, MMP1, COL5A2, IL24, AADACL2, PTCH1, CD68, PRKACA, and SPP1; and a mutational change of TERT, CDKN2A, TP53, or PTCH1, or a combination thereof;
wherein the gene expression levels are detected by contacting the isolated nucleic acids with a set of probes that recognizes at least one but no more than nine genes selected from IGFL1, MMP1, COL5A2, IL24, AADACL2, PTCH1, CD68, PRKACA, and SPP1, and detect binding between the genes and the set of probes.
